(19) United States
(12) Patent Application Publication
(10) Pub. No.: US 2024/0254489 A1
(43) Pub. Date: Aug. 1, 2024

(54) COMPOSITIONS AND METHODS OF TARGETING THE PAX6 SIGNALING PATHWAY TO REDUCE FORMATION OF AMYLOID BETA PLAQUES AND NEUROFIBRILLARY TANGLES

(71) Applicant: The University of Hong Kong, Hong Kong (CN)

(72) Inventors: You-Qiang Song, Hong Kong (CN); Yalun Zhang, Toronto (CA); Yi Zhang, Hong Kong (CN)

(21) Appl. No.: 18/560,347

(22) PCT Filed: May 20, 2022
(86) PCT No.: PCT/CN2022/094180

§ 371 Date: Nov. 10, 2023

Related U.S. Application Data
(60) Provisional application No. 63/191,925, filed on May 21, 2021.

(51) Int. Cl. C12N 15/113; A61K 31/519; A61K 45/06; A61P 25/28

(52) U.S. Cl. CPC C12N 15/113; A61K 31/519; A61K 45/06; A61P 25/28; C12N 2310/14; C12N 2310/531

(57) ABSTRACT

Compositions and method of reducing Tau phosphorylation in neurons of a subject in need thereof are provided. In some embodiments, the subject has a proteinopathy, amyloidosis, or a tauopathy. Thus, compositions and methods of treating a proteinopathy, amyloidosis, or a tauopathy are also provided. Also disclosed are compositions and methods of increasing learning and/or memory in a subject with a tauopathy. The methods typically include administering the subject an effective amount of a direct or indirect inhibitor of Pax6 (Pax6 inhibitor). The Pax6 inhibitor can be, for example, a small molecule or a functional nucleic acid. In some embodiments, the small molecule is palbociclib, apigenin, flavopiridol, abemaciclib, ribociclib, or ICCB280, or a derivative, stereoisomer, or pharmaceutically acceptable salt thereof. In some embodiments, the functional nucleic acid including but not limited to siRNA, shRNA, or miRNA, or a nucleic acid expression construct encoding an siRNA, shRNA, or miRNA.

This international patent application claims the benefit of U.S. Provisional Patent Application No. 63/191,925 filed on May 21, 2021, the entire content of which is incorporated by reference for all purpose.

### FIELD OF THE INVENTION

This invention is generally in the field of compositions and methods of modulating expression of Pax6 and other members in it's signaling pathway, most particularly to treat neurodegeneration.

### BACKGROUND OF THE INVENTION

Genetic and cell biology studies have shown an important role for amyloid β (Aβ) peptide and microtubule-associated protein tau in the pathogenesis of Alzheimer's disease (AD)^{1, 2}. The molecular pathways linking amyloid β, tau and cell death are controversial³⁻⁵. Some evidence from experiments, including in vitro studies of neuronal cell death, in vivo investigation of animal models, and human post-mortem studies, support a hypothesis that implicates deregulation of cell cycle proteins as key mediators of neuronal dysfunction and loss in Alzheimer's disease brains^{6, 7}. For example, cell division cycle 25 (Cdc25) phosphatases have increased expression and activity in Alzheimer's disease brains^{8, 9}. Multiple cyclin-dependent kinases (Cdks; Cdk1, Cdk4 and Cdk6), retinoblastoma protein (pRb), cyclins (A, B, C, D and E) and Cdk inhibitors are overexpressed in cellular and animal models and post-mortem brains of Alzheimer's disease patients¹⁰⁻¹⁹. Central to the hypothesis of the involvement of cell cycle deregulation in neuronal death in Alzheimer's disease is the Cdk/pRb/E2F1 pathway, wherein amyloid β activates Cdk4/6, leading to pRb phosphorylation and activation of E2F1 transcription factor. However, the precise detail of the mechanism by which amyloid β accumulation is linked to neurofibrillary tangles pathology in Alzheimer's disease remains elusive.

It is an object of the invention to provide new targets for reducing amyloid β accumulation and neurofibrillary tangles formation, and compositions and method of use directed thereto.

### SUMMARY OF THE INVENTION

Compositions and method of reducing Tau phosphorylation in neurons of a subject in need thereof are provided. In some embodiments, the subject has a proteinopathy, amyloidosis, or a tauopathy. Thus, compositions and methods of treating a proteinopathy, amyloidosis, or a tauopathy are also provided. As disclosed are compositions and methods of increasing learning and/or memory in a subject with a tauopathy. In some embodiments, the tauopathy is selected from Alzheimer's disease, Frontotemporal lobar degeneration (FTLD), autism, epilepsy, stroke, Dravet syndrome and seizure disorders.

The methods typically include administering the subject an effective amount of a direct or indirect inhibitor of Pax6 (i.e., Pax6 inhibitor). In some embodiments, the amount is effective to reduce the formation amyloid β plaques, reduce the formation of neurofibrillary tangles, or a combination thereof in the subject. In some embodiments, the Pax6 inhibitor is effective to reduce neuronal cell death in the subject.

The Pax6 inhibitor can be, for example, a small molecule or a functional nucleic acid. In some embodiments, the small molecule is palbociclib, flavopiridol, Abemaciclib, Ribociclib or apigenin or ICCB280, or a derivative, stereoisomer, or pharmaceutically acceptable salt thereof. In some embodiments, the functional nucleic acid is selected from the group consisting of antisense molecules, siRNA, shRNA, miRNA, aptamers, ribozymes, triplex forming molecules, RNAi, and external guide sequences that targets the Pax6 gene or a gene product thereof, or an expression construct, such as a plasmid, virus, or viral vector encoding the same. In particular embodiments, the Pax6 inhibitor is an siRNA, shRNA, or miRNA, or G-quadruplex or a nucleic acid expression construct encoding an siRNA, shRNA, or miRNA, optionally wherein the siRNA, shRNA, or miRNA targets any one of SEQ ID NOS:1-7, or a variant thereof with e.g., at least 70% sequence identity thereof, or a nucleic acid encoding the polypeptide of any one of SEQ ID NOS:8-14, or a variant thereof with e.g., at least 70% sequence identity thereof, optionally wherein the nucleic acid expression construct is a plasmid or a virus or viral vector, optionally wherein the virus or viral vector is adeno-associated viruses (AAV). In some embodiments, the miRNA is miR-670, miR-215 and miR-692. In other embodiments, the inhibitor is a small activating RNA (saRNA), such as CEBPA-saRNA.

The Pax6 inhibitor can be targeted to the brain, or cells thereof such as neurons and microglia.

The Pax6 inhibitor can be administered to the subject by an oral, parenteral, transdermal, or transmucosal administration, optionally wherein the transmucosal administration is intranasal. The Pax6 inhibitor can be administered to the subject locally or systemically.

In some embodiments, the inhibitor is packaged in a delivery vehicle such as polymeric microparticles or liposomes.

### BRIEF DESCRIPTION OF THE DRAWINGS

FIGS. 1A-1E illustrate the E2F1 and c-Myb interact with the Pax6 promoter in both mouse and human cells. FIG. 1A is a schematic representation of E2F1 binding sites in the mouse Pax6 promoter region. FIG. 1B is images of electrophoretic gels showing the results of a ChIP assay showing E2F1 binding to the Pax6 promoter in mouse cortical neurons (left) and human HEK293 cells (right). FIG. 1C is a schematic representation of c-Myb binding sites in the mouse Pax6 promoter region. FIG. 1D is images of electrophoretic gels of a ChIP assay showing c-Myb binding to the Pax6 promoter in mouse cortical neurons (left) and human HEK293 cells (right). FIG. 1E is a series of bar graphs showing the results of a Pax6 promoter luciferase assay. A luciferase construct (containing the human wild-type Pax6 promoter) and a Renilla control plasmid were co-transfected into N2a cells with a pcDNA control vector, E2F1 or c-Myb expression plasmid (left panel) or a shRNA control vector, E2F1 shRNA (middle panel) or c-Myb shRNA plasmids (right panel). One day after transfection, cell lysates were processed for both the luciferase and Renilla assay. Data are presented as normalised luciferase activity. Error bars for the figure represent mean±SEM; *p<0.05, ***p<0.001 and ****p<0.0001, n=3; Unpaired two-tailed t-test. Three independent experiments were performed in 1E, and two in 1B and 1D.

FIGS. 2A-2D illustrate Pax6 expression is significantly increased in the entorhinal cortex of TgCRND8 transgenic mice and Alzheimer's disease-post mortem brain. FIGS. 2A and 2B are images of autoradiograms and associated quantitative bar graphs of (2A) Pax6 and (2B) c-Myb Western blots of frontal cortical brain lysates from normal control (lanes 1-7, 15-21; n=14) and Alzheimer's disease tissues (lanes 8-14, 22-28; n=14). GAPDH was used as a loading control. FIG. 2C is a series of immunofluorescent image of brain sections were triple-stained with NeuN antibody (green) anti-Pax6 monoclonal antibody (red) and DAPI for 24 h at 4° C. The sections were incubated with Alexa-488-conjugated antibody specific for rabbit IgG and Alexa-594-conjugated antibody for mouse IgG for 3 h at room temperature. The sections were visualized by confocal microscopy. Scale bar, 20 μm. FIG. 2D is a bar graphs showing the percent of Pax6 positive neurons. NeuN-positive neurons with or without Pax6 staining were counted in layers 2 and 3 of the entorhinal cortex in brains from wild-type and TgCRND8 mice. The counts were obtained by averaging the data from two different experimenters. n=3 mice per group. Error bars for the figure represent mean±SEM; *p<0.05, **p<0.01, ***p<0.001 and ****p<0.0001; Unpaired two-tailed t-test. Representative images are from two or three independent experiments.

FIGS. 3A-3G illustrate Pax6 and c-Myb are upregulated by amyloid β in cultured cortical neurons. FIGS. 3A and 3E are images of electrophoretic gels and associated quantitative bar graphs of Pax6 (3A) and c-Myb (3E) mRNA. β-actin was used as a loading control. Data for Pax6 or c-Myb expression were normalized to β-actin and to the zero time point, and were compared to untreated controls. FIGS. 3B and 3F are images of autoradiograms and associated quantitative bar graphs Pax6 (3B) and c-Myb (3F) proteins (Western blots). β-actin was used as the loading control. FIGS. 3C, 3D, and 3G are bar graphs showing (3C) Pax6 transcription activity, (3D) Pax6 promoter activation by amyloid β treatment, (3G) c-Myb transcriptional activity. Error bars for the figure represent mean±SEM; *p<0.05, **p<0.01, ***p<0.001 and ****p<0.0001, n=3; One-way ANOVA with Tukey's multiple comparison test. All data are from at least three independent experiments.

FIGS. 4A-4L illustrate that Cdk/E2F1 regulates Pax6 expression and activity in an amyloid β model. FIG. 4A is images of electrophoretic gels and the associated quantitative bar graphs showing the affect of Pax6 and c-Myb by siRNA protect cortical neurons from death induced by amyloid β treatment. Cortical neurons were transfected with two Pax6 (left) or two c-Myb (right) siRNA oligonucleotides (siPax6-1 or siPax6-2, sic-Myb1 or sic-Myb2) or control siRNA (si-Control) for 24 h and then treated with amyloid β₁₋₄₂ for 36 and 48 h. FIG. 4B is a bar graphs showing the results of a survival assay of amyloid β-induced death of cortical neurons co-treated with flavopiridol. FIG. 4C is an image of an electrophoretic gel and the associated quantitative bar graphs showing the affect of Cdk inhibition with flavopiridol decreases amyloid β-induced Pax6 and c-Myb mRNA upregulation (left). Densitometric analysis of mRNA levels (right). FIG. 4D is an image of an autoradiogram and the associated quantitative bar graphs showing the affect of Cdk inhibition with flavopiridol on amyloid β-induced upregulation of Pax6, c-Myb and E2F1 protein levels in cortical neurons. β-actin levels are used as a control for equal input. FIG. 4E is a bar graph showing the affect of Cdk inhibition with flavopiridol on amyloid β-induced activation of the Pax6 promoter. FIG. 4F is an image of an electrophoretic gel and corresponding quantitative bar graphs showing the results of E2F1 knockdown on amyloid β-induced upregulation of c-Myb and Pax6 at the mRNA level. Cortical neurons were transfected with a control siRNA (si-Control) or E2F1 siRNA oligonucleotides (siE2F1) for 24 h, followed by treatment with amyloid β₁₋₄₂ for 12 h and 24 h. FIG. 4G is an image of an autoradiogram and corresponding quantitative bar graphs showing the results of E2F1 knockdown on amyloid β-induced upregulation of c-Myb and Pax6 at the protein level. β-actin was used as a loading control. FIG. 4H is an image of an electrophoretic gel and corresponding quantitative bar graphs showing the results of a ChIP assay showing that amyloid β enhances E2F1 occupancy of the Pax6 promoter. FIGS. 4I and 4J is an image of an electrophoretic gel and corresponding quantitative bar graph (4I) and an image of an autoradiogram and corresponding quantitative bar graph (4J) showing the results of (4I) RT-PCR and (4J) Western blot showing siRNA inhibition of c-Myb. β-actin was used as a loading control. FIG. 4K is an image of an electrophoretic gel and corresponding quantitative bar graph showing the affect of Amyloid β treatment on occupancy of the Pax6 promoter by c-Myb. FIG. 4L is an image of an electrophoretic gel and corresponding quantitative bar graph showing the affect of E2F1 knockdown on occupancy of the Pax6 promoter by c-Myb upon amyloid β treatment. Error bars for the figure represent mean±SEM; *p<0.05, **p<0.01, ***p<0.001, and ****p<0.0001, n=3; One-way ANOVA with Tukey's multiple comparison test. All data are representative of at least three independent experiments.

FIGS. 5A-5G illustrate that Pax6 transactivates GSK-3β and regulates tau phosphorylation in amyloid β signaling. FIG. 5A is images of electrophoretic gels showing the results of a ChIP assay showing Pax6 binding to the GSK-3β promoter in mouse cortical neurons (left) and HEK293 cells (right). FIG. 5B is an image of an electrophoretic gel and the associated quantitative bar graph showing the results of a ChIP assay showing the affect of amyloid β treatment on occupancy of the GSK-3β promoter by Pax6 (upper) confirmed by densitometric analysis (lower). FIGS. 5C and 5D is an image of an electrophoretic gel (5C) or autoradiogram (5D) and the associated quantitative bar graphs showing the results of an RT-PCR showing GSK-3β mRNA levels and (5D) Western blot showing GSK-3β protein (5D) after amyloid β treatment. FIGS. 5E and 5F is an image of an electrophoretic gel (5F) or autoradiogram (5G) and the associated quantitative bar graphs showing the results of an RT-PCR (5E) and Western blot (5F) showing the affect of inhibition of Pax6 by siRNA on amyloid β-induced increase of GSK-3β mRNA and protein. FIG. 5G is an image of an autoradiogram of a Western blot showing the affect of Pax6 by siRNA on amyloid β-induced total tau and Ser356, Ser396 and Ser404 phosphorylation. Relative increase in tau phosphorylation was normalized against tau5. β-actin was used as a loading control. Error bar for the figure represents mean±SEM, *p<0.05, **p<0.01, ***p<0.001 and ****p<0.0001; P values were calculated by one-way ANOVA with Tukey's multiple comparison test in 5A-5G. Data are representative of at least three independent experiments.

FIG. 6 is an illustration of a model for the role of Pax6 in amyloid β induced neurotoxicity.

FIG. 7A is an image of an autoradiogram and the associated quantitative bar graph showing the results of an immunoblotting assay used to characterize the expression level of Pax6 in hippocampus of 9-month old WT mice and 5×FAD mice. FIG. 7B is a schematic representation of Pax6 therapeutic delivery and experimental time course for the experiment described in Example 9. 3-month old male 5×FAD mice were stereotaxically injected with AAVs (4 μl; 1.3×10¹³ GC/ml), 3 months post injection, mice were subjected to Morris water maze. FIGS. 7C-7F are a line graph (7C) and bar graphs (7D-7F) showing the results of a Morris water maze test on 5×FAD mice administered Pax6 shRNA AAV or scrambled shRNA AAV, or left untreated.

FIGS. 8A-8C illustration palbociclib inhibition of 293T-Tau and 293T-APP cell lines. FIG. 8A is a dot plot showing the results of an XTT assay for choosing effective concentrations of Palbociclib. FIGS. 8B and 8C are images of autoradiograms of Western blots and the associated quantitative bar graphs showing expression levels of CDK4 and Cyclin D1 by adding palbociclib (10 nM and 50 nM) in 293T-Tau (8B); 293T-APP (8C) cell lines.

FIG. 9A-9D show effects of palbociclib on 5×FAD mice model. FIGS. 9A and 9B is a series of immunofluorescent images showing expression of cell cycle marker (Cyclin D1) (9A) and Pax6 (9B). FIG. 9C is a schematic of a strategy for using palbociclib as a treatment on 5×FAD mice models and demonstrated that palbociclib is able to improve memory and learning of 5×FAD mice. FIG. 9D is an image of an autoradiogram of a Western blot and the associated quantitative bar graph showing the relative expression of E2F-1, CDK6, c-Myb, APP, Iba1 and LC3B in palbociclib treatment group compared to controls. FIG. 9E is a series of images of brain tissue showing the relative Aβ deposits in palbociclib treated mouse brain tissue compared to controls. FIG. 9F is a series of images of liver, gallbladder and kidney in palbociclib treated mouse. FIGS. 9G-9J show the effect of palbociclib treatment on learning and memory ability of 5×FAD female mice in morris water maze. FIG. 9G is a schematic diagram of palbociclib administration with 60 mg/(kg*d); 10-month-old wild-type mice (WT) and 5×FAD mice were administrated with solvent (ctrl) and 60 mg/kg/day palbociclib (palb) for 2 months via oral garage; Morris water maze with n(WT-ctrl)=5, n(5×FAD-ctrl)=5, n(5×FAD-palb)=3 were performed. FIG. 9H shows latency to platform in training trial tested and analyzed by two-way ANOVA test. FIG. 9I shows latency to platform in probe trial of the palbocilib treatment group was significantly improved to the level of the wild type control. FIG. 9J shows frequency of crossing platform in probe trial was significantly increased in palbocilib treatment group (ns: not significant, *P<0.05, **P<0.01. One-way ANOVA).

FIGS. 10A-10C show the identification of downstream regulation genes of E2F1. FIG. 10A is a workflow of data processing. FIG. 10B is a volcano plot of the differential expression between Alzheimer's disease and healthy brains. The light blue points represent DEGs selected on the basis of FC>1.5 and adjusted p-value<0.05. The dark blue points represent those DEGs predicted to be potential E2F1 targets. The yellow points indicate brain marker genes potentially targeted by E2F1 and differentially expressed. The red points represent genes with no significant difference. FIG. 10C is a series of heatmap s of the three GO semantic similarity scores of the selected brain markers and the overall relevance. The overall relevance represents the arithmetic average value of the three GO semantic similarity scores (BP, MF and CC) between E2F1 and the selected genes. Dark colours indicate high similarity and light colours indicate low similarity. DEGs=differentially expressed genes. FC=fold change. GO=gene ontology. BP=biological process. MF=molecular function. CC=cellular component.

FIGS. 11A and 11B are plots showing Pax6 expression during different stages (11A) and brain regions (11B) in subjects with Alzheimer's disease.

FIG. 12A is a volcano plot of the differential expression between Alzheimer's disease and healthy brains (Differential gene expression in FPKM of all genes involved in Alzheimer's disease-related enriched KEGG pathways. Genes with predicted Pax6 binding sites are shown by red dots). FIG. 12B is a bar graphs showing fold changes of gene expression for selected genes after Pax6 knockdown. FIG. 12C is a bar graphs showing Pax6 and GSK-3β gene expression in FPKM value after Pax6 knockdown. FPKM=Fragments Per Kilobase of exon model per Million mapped fragments.

FIGS. 13A-13D are images of electrophoretic gels and the accompanying quantitation in bar graphs showing the transactivation activity mediated by Pax6 (13A), c-Myb (13B) and E2F1 (13C and 13D) in Alzheimer's disease brain. Chromatin was extracted from 10 Alzheimer's disease cases and 10 controls of human frontal cortex tissues. Chromatin immunoprecipitations were performed with anti-Pax6, anti-c-Myb or anti-E2F1 antibodies. IgG was used as negative control. The eluted materials were purified and amplified in RT-PCR. ChTP assay showed occupancy of GSK-3β promoter binding by Pax6 (13A), Pax6 promoter binding by c-Myb (13B), c-Myb promoter binding by E2F1 (13C), Pax6 promoter binding by E2F1 (13D) increased in Alzheimer's disease brain compared with control. Error bars for the figure represent mean SEM; **p<0.01; Unpaired two-tailed t-test; n=10 per group.

FIGS. 14A-14D are survival plots showing the results of vab-3 (C. elegans ortholog of human Pax6) RNAi compared to vehicle control in N2, CL2355, CK12, and BR5270 C. elegans. See also Table 5.

### DETAILED DESCRIPTION OF THE INVENTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “inhibit” or other forms of the word such as “inhibiting” or “inhibition” means to hinder or restrain a particular characteristic. It is understood that this is typically in relation to some standard or expected value, in other words it is relative, but that it is not always necessary for the standard or relative value to be referred to. For example, “inhibit” can mean hindering or restraining the activity of a protein, hindering or restraining the synthesis or expression of a protein, or mRNA encoding the protein, relative to a standard or control.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, the term “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, the term “localization signal or sequence or domain or ligand” or “targeting signal or sequence or domain or ligand” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

As used herein, the term “microparticles” refers to particles having a diameter between one micron and 1000 microns, typically less than 400 microns, more typically less than 100 microns, most preferably for the uses described herein in the range of less than 10 microns in diameter. Microparticles include microcapsules and microspheres unless otherwise specified.

As used herein, the term “nanoparticles” refers to particles having a diameter of less than one micron, more typically between 50 and 1000 nanometers, preferably in the range of 100 to 300 nanometers.

### II. Methods of Use

Alzheimer's disease (AD), a chronic neurodegenerative disorder, is characterized by cognitive dysfunction and memory loss. However, the mechanisms underlying two hallmark pathological features of Alzheimer's disease, amyloid β plaques and neurofibrillary tangles, has remained elusive. The experiments in the Examples below show that Pax6 is a key regulator to link amyloid β to Tau phosphorylation. The upregulation of Pax6 expression is observed in human and mouse AD brain. In vitro studies show that amyloid β activates the cell cycle mediators CDKs/E2F1, which leads to the induction of c-Myb and Pax6, Pax6 is downstream target of both E2F1 and c-Myb. Furthermore, the results show that Pax6 regulates one of key kinases that phosphorylate Tau, GSK-3β. Pax6 silenced by siRNA decreases the tau phosphorylation of Ser356, Ser396, and Ser404, and results in the 5×FAD mouse model of Alzheimer's disease shows that two cyclin-dependent kinase (CDK) inhibitor, such as palbociclib, can modulate the Pax6 pathway.

Disclosed herein are compositions and methods of use thereof for directly and indirectly modulating the Pax6 signaling pathway. Preferably, the compositions are effective to reduce Tau phosphorylation, reduce in the formation amyloid β plaques, reduce the formation of neurofibrillary tangles, reduce neuronal cell death, reduce one or more symptoms of a neurological disorder, and/or another medical, biochemical, or physiological endpoint discussed herein. Formulations for modulating the Pax6 signaling pathway are also provided.

### A. Methods of Inhibiting Pax6 Signaling

The experiments below show that reducing expression of Pax6 or otherwise reducing its activity or bioavailable is effective in reducing Tau phosphorylation or total Tau and/or neuronal cell death. Thus, the disclosed methods typically include administering a subject in need thereof an effective amount of a composition to reduce expression, activity, and/or bioavailability Pax6, by directly inhibiting its expression, activity, or bioavailability. Additionally or alternatively, Pax6 expression, activity, and/or bioavailability can be indirectly reduced by inhibiting the expression, activity, or bioavailability of one or more of its transctivators including, but not limited to, Cdk, pRb, E2F1 and/or c-Myb. Additionally or alternatively, downstream targets of Pax6, including but not limited to, Cdk5 and p35, GSK-3β, mitogen-activated protein kinases (MAPK), serine/threonine protein kinase (MARK), calcium/calmodulin-dependent protein kinase type II a (CAMK2a), etc. are indirectly or directly inhibited, preferably leading to reduced phosphorylation of Tau, optionally at Ser356, Ser396, and/or Ser404. Down pax6 expression will increase BDNF expression and has a neurprotective effect. Moreover, down pax6 expression will also increase TREM2 expression to regulate neuroinflammatiuon.

In some embodiments, the effect of the disclosed compositions and methods on a subject is compared to a control. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known the art, such as one of those discussed herein. The subject can be administered the composition(s) in a dosage and for a duration sufficient to accomplish the intend effect compared to a control.

The route of administration can be oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In certain embodiments, the compositions are administered systemically or locally, for example by injection or other application directly into or onto a site to be treated. Typically, local administration causes an increased localized concentration of the compositions which is greater than that which can be achieved by systemic administration.

The precise dosage will vary according to a variety of factors including but not limited to the inhibitor that is selected and subject-dependent variables (e.g., age, immune system health, clinical symptoms etc.).

The timing of the administration of the composition will also depend on the formulation and/or route of administration used. The compound may be administered once daily, but may also be administered two, three or four times daily, or every other day, or once or twice per week. For example, the subject can be administered one or more treatments 1, 2, 3, 4, 5, 6, 7, 8, 9, 10, 11, 12, 13, 14, 15, 16, 17, 18, 19, 20, 21, 22, 23, or 24 hours, days, weeks, or months apart.

In some embodiments, the compositions are formulated for extended release. For example, the formulation can be suitable for administration once daily or less. In some embodiments, the composition is only administered to the subject once every 24-48 hours.

In some embodiments, administration of the composition will be given as a long-term treatment regimen whereby pharmacokinetic steady state conditions will be reached.

### B. Conditions to be Treated

The compositions disclosed herein can be used to prevent, reduce, delay, or inhibit the formation or aggregation of misfolded proteins; prevent, reduce, delay, or inhibit the level, formation, or production of amyloid proteins, such as amyloid beta, in a subject over time; prevent, reduce, delay, or inhibit the expression of or phosphorylation of Tau and/or total Tau in a subject over time; or a combination thereof. In some embodiments, the composition prevent, reduce, delay, or inhibit the level of amyloid-mediated and/or Tau-mediate neuronal cell death. The compositions are particularly useful for treating a subject with, or likely to develop, a proteinopathy, amyloidosis, or a tauopathy. Additionally or alternatively, in some embodiments, the composition is administered in an effective amount to reduce one or more symptoms of the disease or disorder, such as the proteinopathy, amyloidosis, or a tauopathy.

In some embodiments provide compositions and methods to treat a disease characterized by increased amyloid beta expression, deposition, aggregation or plaque formation. For example, a method of treating a disease or disorder characterized by increased amyloid beta expression, deposition, aggregation or plaque formation can include administering to a subject in need thereof an effective amount of a composition to reduce, delay, or inhibit the level, formation, or production of amyloid beta in the subject compared to a control.

Abeta-related diseases and disorders include, but are not limited to, Alzheimer's disease, cerebral amyloid angiopathy (also known as congophilic angiopathy), Lewy body dementia, retinal ganglion cell degeneration (such as in glaucoma), sporadic inclusion body myositis (sIBM) and hereditary inclusion body myopathy (hIBM).

The abeta-related disease or diseases treated using the disclosed method is not Alzheimer's disease or Lewy body dementia.

Abeta is formed after sequential cleavage of the amyloid precursor protein (APP), a transmembrane glycoprotein of undetermined function. APP can be processed by α-, β- and γ-secretases; Abeta protein is generated by successive action of the β and γ secretases. The γ secretase, which produces the C-terminal end of the Abeta peptide, cleaves within the transmembrane region of APP and can generate a number of isoforms of between 36 and 43 amino acid residues in length. The most common isoforms are Abeta40 and Abeta42; the longer form is typically produced by cleavage that occurs in the endoplasmic reticulum, while the shorter form is produced by cleavage in the trans-Golgi network. The Abeta40 form is the more common of the two, but Abeta42 is the more fibrillogenic and is thus associated with disease states.

The disclosed compositions and methods can also be used to treat diseases characterized by increased tau expression, increased tau phosphorylation, or pathologies associated with the aggregation of tau protein in the brain. For example, a method of treating a disease or disorder characterized by increased tau expression, increased tau phosphorylation, or pathologies associated with the aggregation of tau protein in the brain can include administering to a subject in need thereof an effective amount of a composition to reduce, delay, or inhibit the expression of or phosphorylation of tau in the subject compared to a control.

Examples of tauopathies and conditions associated therewith include, but are not limited to Alzheimer's disease, Argyrophilic grain disease (AGD), Chronic Traumatic Encephalopathy (CTE), Dementia pugilistica (chronic traumatic encephalopathy), frontotemporal dementia, frontotemporal lobar degeneration.gangliocytoma, Ganglioglioma, gangliocytoma, Lytico-Bodig disease (Parkinson-dementia complex of Guam), meningioangiomatosis, Frontotemporal dementia and Parkinsonism linked to chromosome 17 (FTDP-17), Pick's disease, Progressive supranuclear palsy, subacute sclerosing panencephalitis, tangle-predominant dementia, lead encephalopathy, tuberous sclerosis, Hallervorden-Spatz disease, lipofuscinosis, corticobasal degeneration.

In some embodiments the taupathy is a non-Alzheimer's tauopathy. Non-Alzheimer's tauopathies are sometimes grouped together as “Pick's complex.”

There is a subgroup of neurodegenerative disorders having an important feature of tauopathy, of which reduced total tau level will prevent these disorders including some Alzheimer's disease, some Frontotemporal lobar degeneration (FTLD), some autism, some depression, some epilepsy, some stroke, some traumatic spinal cord injury (SCI), some Dravet syndrome and some seizures. See, e.g., Chang, et al., “Tau: Enabler of diverse brain disorders and target of rapidly evolving therapeutic strategies”, Science 26 Feb. 2021: Vol. 371, Issue 6532, eabb8255, DOI: 10.1126/science.abb8255; Tai, et al., “Tau Reduction Prevents Key Features of Autism in Mouse Models”, Neuron. 2020 May 6; 106(3):421-437.e11. doi: 10.1016/j.neuron.2020.01.038. Epub 2020 Mar. 2; Sotiropoulos, et al., “Atypical, non-standard functions of the microtubule associated Tau protein,” Acta Neuropathol Commun. 2017 Nov. 29; 5(1):91. doi: 10.1186/s40478-017-0489-6; Bi, et al., “Tau exacerbates excitotoxic brain damage in an animal model of stroke,” Nat Commun., 2017 Sep. 7; 8(1):473. doi: 10.1038/s41467-017-00618-0; Gheyara, et al., “Tau reduction prevents disease in a mouse model of Dravet syndrome,” Ann Neurol., 2014 September; 76(3):443-56. doi: 10.1002/ana.24230. Epub 2014 Aug. 13; Roberson, et al., “Reducing endogenous tau ameliorates amyloid beta-induced deficits in an Alzheimer's disease mouse model,” Science, 2007 May 4; 316(5825):750-4. doi: 10.1126/science.1141736; Ittner, et al., “Dendritic function of tau mediates amyloid-beta toxicity in Alzheimer's disease mouse models,” Cell., 2010 Aug. 6; 142(3):387-97. doi: 10.1016/j.cell.2010.06.036. Epub 2010 Jul. 22; DeVos, et al., “Antisense reduction of tau in adult mice protects against seizures,” J Neurosci. 2013 Jul. 31; 33(31):12887-97. doi: 10.1523/JNEUROSCI.2107-13.2013; Caprelli, et al., “Hyperphosphorylated Tau as a Novel Biomarker for Traumatic Axonal Injury in the Spinal Cord,” J Neurotrauma, 2018 Aug. 15; 35(16):1929-1941. doi: 10.1089/neu.2017.5495; Caprelli, et al., CNS Injury: Posttranslational Modification of the Tau Protein as a Biomarker,” Neuroscientist, 2019 February; 25(1):8-21. doi: 10.1177/1073858417742125. Epub 2017 Nov. 22; Yang, et al., “Involvement of tau phosphorylation in traumatic brain injury patients,” Acta Neurol Scand., 2017 June; 135(6):622-627. doi: 10.1111/ane.12644. Epub 2016 Jul. 21.

The experiments herein demonstrate that down regulation of Pax6 will reduce total tau level in neurones. It is believed that inhibition of Pax6 will prevent these tauopathy disorders in including the foregoing (e.g., Alzheimer's disease, FTLD, autism, epilepsy, stroke, Dravet syndrome and seizures, etc.) Thus, in some embodiments, the subject has one of these diseases or conditions.

### III. Compositions

### A. Sequences

### 1. Pax6 Sequences

Nucleic acid and amino acid sequences for Pax6 are known in the art. See, non-limiting examples, NCBI Reference Sequences, NM_000280, NM_001258462, NM_001258463, NM_001258464, NM_001276122, and NC_004353.4, and Gene ID: 43812 and Gene ID: 43833 each of which is specifically incorporated by references herein, and including all of the sequence provided, cited, and referenced therein.

| | |
|---|---|
| 1 | agcataggaa tctgagaatt gctctcacac accaacccag caacatccgt ggagaaaact |
| 61 | ctcaccagca actcctttaa aacaccgtca tttcaaacca ttgtggtctt caagcaacaa |
| 121 | cagcagcaca aaaaacccca accaaacaaa actcttgaca gaagctgtga caaccagaaa |
| 181 | ggatgcctca taaaggggga agactttaac taggggcgcg cagatgtgtg aggcctttta |
| 241 | ttgtgagagt ggacagacat ccgagatttc agagccccat attcgagccc cgtggaatcc |
| 301 | cgcggccccc agccagagcc agcatgcaga acagtcacag cggagtgaat cagctcggtg |
| 361 | gtgtctttgt caacgggcgg ccactgccgg actccacccg gcagaagatt gtagagctag |
| 421 | ctcacagcgg ggcccggccg tgcgacattt cccgaattct gcaggtgtcc aacggatgtg |
| 481 | tgagtaaaat tctgggcagg tattacgaga ctggctccat cagacccagg gcaatcggtg |
| 541 | gtagtaaacc gagagtagcg actccagaag ttgtaagcaa aatagcccag tataagcggg |
| 601 | agtgcccgtc catctttgct tgggaaatcc gagacagatt actgtccgag ggggtctgta |
| 661 | ccaacgataa cataccaagc gtgtcatcaa taaacagagt tcttcgcaac ctggctagcg |
| 721 | aaaagcaaca gatgggcgca gacggcatgt atgataaact aaggatgttg aacgggcaga |
| 781 | ccggaagctg gggcacccgc cctggttggt atccggggac ttcggtgcca gggcaaccta |
| 841 | cgcaagatgg ctgccagcaa caggaaggag ggggagagaa taccaactcc atcagttcca |
| 901 | acggagaaga ttcagatgag gctcaaatgc gacttcagct gaagcggaag ctgcaaagaa |
| 961 | atagaacatc ctttacccaa gagcaaattg aggccctgga gaaagagttt gagagaaccc |
| 1021 | attatccaga tgtgtttgcc cgagaaagac tagcagccaa aatagatcta cctgaagcaa |
| 1081 | gaatacaggt atggttttct aatcgaaggg ccaaatggag aagagaagaa aaactgagga |
| 1141 | atcagagaag acaggccagc aacacaccta gtcatattcc tatcagcagt agtttcagca |
| 1201 | ccagtgtcta ccaaccaatt ccacaaccca ccacaccggt ttcctccttc acatctggct |
| 1261 | ccatgttggg ccgaacagac acagccctca caaacaccta cagcgctctg ccgcctatgc |
| 1321 | ccagcttcac catggcaaat aacctgccta tgcaaccccc agtccccagc cagacctcct |
| 1381 | catactcctg catgctgccc accagccctt cggtgaatgg gcggagttat gatacctaca |
| 1441 | cccccccaca tatgcagaca cacatgaaca gtcagccaat gggcacctcg ggcaccactt |
| 1501 | caacaggact catttcccct ggtgtgtcag ttccagttca agttcccgga agtgaacctg |
| 1561 | atatgtctca atactggcca agattacagt aaaaaaaaaa aaaaaaaaaa aaaggaaagg |
| 1621 | aaatattgtg ttaattcagt cagtgactat ggggacacaa cagttgagct ttcaggaaag |
| 1681 | aaagaaaaat ggctgttaga gccgcttcag ttctacaatt gtgtcctgta ttgtaccact |
| 1741 | ggggaaggaa tggacttgaa acaaggacct ttgtatacag aaggcacgat atcagttgga |
| 1801 | acaaatcttc attttggtat ccaaactttt attcattttg gtgtattatt tgtaaatggg |
| 1861 | catttgtatg ttataatgaa aaaaagaaca atgtagactg gatggatgtt tgatctgtgt |
| 1921 | tggtcatgaa gttgtttttt ttttttttaa aaagaaaacc atgatcaaca agctttgcca |
| 1981 | cgaatttaag agttttatca agatatatcg aatacttcta cccatctgtt catagtttat |
| 2041 | ggactgatgt tccaagtttg tatcattcct ttgcatataa ttaaacctgg aacaacatgc |
| 2101 | actagattta tgtcagaaat atctgttggt tttccaaagg ttgttaacag atgaagttta |
| 2161 | tgtgcaaaaa agggtaagat ataaattcaa ggaagaaaaa aagttgatag ctaaaaggta |
| 2221 | gagtgtgtct tcgatataat ccaatttgtt ttatgtcaaa atgtaagtat ttgtcttccc |
| 2281 | tagaaatcct cagaatgatt tctataataa agttaatttc atttatattt gacaagaata |
| 2341 | tagatgtttt atacacattt tcatgcaatc atacgtttct tttttggcca gcaaaagtta |
| 2401 | attgttctta gatatagttg tattactgtt cacggtccaa tcattttgtg catctagagt |
| 2461 | tcattcctaa tcaattaaaa gtgcttgcaa gagttttaaa |

(SEQ ID NO: 1) (Homo sapiens paired box 6 (PAX6), transcript variant 1,
mRNA
NCBI Reference Sequence: NM_000280.5), which encodes a polypeptide
having the amino acid sequence:
MQNSHSGVNQLGGVFVNGRPLPDSTRQKIVELAHSGARPCDISRILQVSNGCVSKILGRYYETGSIRPRAI
GGSKPRVATPEVVSKIAQYKRECPSIFAWEIRDRLLSEGVCTNDNIPSVSSINRVLRNLASEKQQMGADGM
YDKLRMLNGQTGSWGTRPGWYPGTSVPGQPTQDGCQQQEGGGENTNSISSNGEDSDEAQMRLQLKRKLQRN
RTSFTQEQIEALEKEFERTHYPDVFARERLAAKIDLPEARIQVWFSNRRAKWRREEKLRNQRRQASNTPSH
IPISSSFSTSVYQPIPQPTTPVSSFTSGSMLGRTDTALTNTYSALPPMPSFTMANNLPMQPPVPSQTSSYS
CMLPTSPSVNGRSYDTYTPPHMQTHMNSQPMGTSGTTSTGLISPGVSVPVQVPGSEPDMSQYWPRLQ
(SEQ ID NO: 8)

| | |
|---|---|
| 1 | accctctttt cttatcattg acatttaaac tctggggcag gtcctcgcgt agaacgcggc |
| 61 | tgtcagatct gccacttccc ctgccgagcg gcggtgagaa gtgtgggaac cggcgctgcc |
| 121 | aggctcacct gcctccccgc cctccgctcc caggaatctg agaattgctc tcacacacca |
| 181 | acccagcaac atccgtggag aaaactctca ccagcaactc ctttaaaaca ccgtcatttc |
| 241 | aaaccattgt ggtcttcaag caacaacagc agcacaaaaa accccaacca aacaaaactc |
| 301 | ttgacagaag ctgtgacaac cagaaaggat gcctcataaa gggggaagac tttaactagg |
| 361 | ggcgcgcaga tgtgtgaggc cttttattgt gagagtggac agacatccga gatttcagag |
| 421 | ccccatattc gagccccgtg gaatcccgcg gcccccagcc agagccagca tgcagaacag |
| 481 | tcacagcgga gtgaatcagc tcggtggtgt ctttgtcaac gggcggccac tgccggactc |
| 541 | cacccggcag aagattgtag agctagctca cagcggggcc cggccgtgcg acatttcccg |
| 601 | aattctgcag acccatgcag atgcaaaagt ccaagtgctg gacaatcaaa acgtgtccaa |
| 661 | cggatgtgtg agtaaaattc tgggcaggta ttacgagact ggctccatca gacccagggc |
| 721 | aatcggtggt agtaaaccga gagtagcgac tccagaagtt gtaagcaaaa tagcccagta |
| 781 | taagcgggag tgcccgtcca tctttgcttg ggaaatccga gacagattac tgtccgaggg |
| 841 | ggtctgtacc aacgataaca taccaagcgt gtcatcaata aacagagttc ttcgcaacct |
| 901 | ggctagcgaa aagcaacaga tgggcgcaga cggcatgtat gataaactaa ggatgttgaa |
| 961 | cgggcagacc ggaagctggg gcacccgccc tggttggtat ccggggactt cggtgccagg |
| 1021 | gcaacctacg caagatggct gccagcaaca ggaaggaggg ggagagaata ccaactccat |
| 1081 | cagttccaac ggagaagatt cagatgaggc tcaaatgcga cttcagctga agcggaagct |
| 1141 | gcaaagaaat agaacatcct ttacccaaga gcaaattgag gccctggaga aagagtttga |
| 1201 | gagaacccat tatccagatg tgtttgcccg agaaagacta gcagccaaaa tagatctacc |
| 1261 | tgaagcaaga atacaggtat ggttttctaa tcgaagggcc aaatggagaa gagaagaaaa |
| 1321 | actgaggaat cagagaagac aggccagcaa cacacctagt catattccta tcagcagtag |
| 1381 | tttcagcacc agtgtctacc aaccaattcc acaacccacc acaccggttt cctccttcac |
| 1441 | atctggctcc atgttgggcc gaacagacac agccctcaca aacacctaca gcgctctgcc |
| 1501 | gcctatgccc agcttcacca tggcaaataa cctgcctatg caacccccag tccccagcca |
| 1561 | gacctcctca tactcctgca tgctgcccac cagcccttcg gtgaatgggc ggagttatga |
| 1621 | tacctacacc cccccacata tgcagacaca catgaacagt cagccaatgg gcacctcggg |
| 1681 | caccacttca acaggactca tttcccctgg tgtgtcagtt ccagttcaag ttcccggaag |
| 1741 | tgaacctgat atgtctcaat actggccaag attacagtaa aaaaaaaaaa aaaaaaaaaa |
| 1801 | aggaaaggaa atattgtgtt aattcagtca gtgactatgg ggacacaaca gttgagcttt |
| 1861 | caggaaagaa agaaaaatgg ctgttagagc cgcttcagtt ctacaattgt gtcctgtatt |
| 1921 | gtaccactgg ggaaggaatg gacttgaaac aaggaccttt gtatacagaa ggcacgatat |
| 1981 | cagttggaac aaatcttcat tttggtatcc aaacttttat tcattttggt gtattatttg |
| 2041 | taaatgggca tttgtatgtt ataatgaaaa aaagaacaat gtagactgga tggatgtttg |
| 2101 | atctgtgttg gtcatgaagt tgtttttttt ttttttaaaa agaaaaccat gatcaacaag |
| 2161 | ctttgccacg aatttaagag ttttatcaag atatatcgaa tacttctacc catctgttca |
| 2221 | tagtttatgg actgatgttc caagtttgta tcattccttt gcatataatt aaacctggaa |
| 2281 | caacatgcac tagatttatg tcagaaatat ctgttggttt tccaaaggtt gttaacagat |
| 2341 | gaagtttatg tgcaaaaaag ggtaagatat aaattcaagg aagaaaaaaa gttgatagct |
| 2401 | aaaaggtaga gtgtgtcttc gatataatcc aatttgtttt atgtcaaaat gtaagtattt |
| 2461 | gtcttcccta gaaatcctca gaatgatttc tataataaag ttaatttcat ttatatttga |
| 2521 | caagaatata gatgttttat acacattttc atgcaatcat acgtttcttt tttggccagc |
| 2581 | aaaagttaat tgttcttaga tatagttgta ttactgttca cggtccaatc attttgtgca |
| 2641 | tctagagttc attcctaatc aattaaaagt gcttgcaaga gttttaaact taagtgtttt |
| 2701 | gaagttgttc acaactacat atcaaaatta accattgttg attgtaaaaa accatgccaa |
| 2761 | agcctttgta tttcctttat tatacagttt tctttttaac cttatagtgt ggtgttacaa |
| 2821 | attttatttc catgttagat caacattcta aaccaatggt tactttcaca cacactctgt |
| 2881 | tttacatcct gatgatcctt aaaaaataat ccttatagat accataaatc aaaaacgtgt |
| 2941 | tagaaaaaaa ttccacttac agcagggtgt agatctgtgc ccatttatac ccacaacata |
| 3001 | tatacaaaat ggtaacattt cccagttagc catttaattc taaagctcaa agtctagaaa |
| 3061 | taatttaaaa atgcaacaag cgattagcta ggaattgttt tttgaattag gactggcatt |
| 3121 | ttcaatctgg gcagatttcc attgtcagcc tatttcaaca atgatttcac tgaagtatat |
| 3181 | tcaaaagtag atttcttaaa ggagactttc tgaaagctgt tgcctttttc aaataggccc |
| 3241 | tctccctttt ctgtctccct cccctttgca caagaggcat catttcccat tgaaccacta |
| 3301 | cagctgttcc catttgaatc ttgctttctg tgcggttgtg gatggttgga gggtggaggg |
| 3361 | gggatgttgc atgtcaagga ataatgagca cagacacatc aacagacaac aacaaagcag |
| 3421 | actgtgactg gccggtggga attaaaggcc ttcagtcatt ggcagcttaa gccaaacatt |
| 3481 | cccaaatcta tgaagcaggg cccattgttg gtcagttgtt atttgcaatg aagcacagtt |
| 3541 | ctgatcatgt ttaaagtgga ggcacgcagg gcaggagtgc ttgagcccaa gcaaaggatg |
| 3601 | gaaaaaaata agcctttgtt gggtaaaaaa ggactgtctg agactttcat ttgttctgtg |
| 3661 | caacatataa gtcaatacag ataagtcttc ctctgcaaac ttcactaaaa agcctggggg |
| 3721 | ttctggcagt ctagattaaa atgcttgcac atgcagaaac ctctggggac aaagacacac |
| 3781 | ttccactgaa ttatactctg ctttaaaaaa atccccaaaa gcaaatgatc agaaatgtag |
| 3841 | aaattaatgg aaggatttaa acatgacctt ctcgttcaat atctactgtt ttttagttaa |
| 3901 | ggaattactt gtgaacagat aattgagatt cattgctccg gcatgaaata tactaataat |
| 3961 | tttattccac cagagttgct gcacatttgg agacaccttc ctaagttgca gtttttgtat |
| 4021 | gtgtgcatgt agttttgttc agtgtcagcc tgcactgcac agcagcacat ttctgcaggg |
| 4081 | gagtgagcac acatacgcac tgttggtaca attgccggtg cagacatttc tacctcctga |
| 4141 | cattttgcag cctacattcc ctgagggctg tgtgctgagg gaactgtcag agaagggcta |
| 4201 | tgtgggagtg catgccacag ctgctggctg gcttacttct tccttctcgc tggctgtaat |
| 4261 | ttccaccacg gtcaggcagc cagttccggc ccacggttct gttgtgtaga cagcagagac |
| 4321 | tttggagacc cggatgtcgc acgccaggtg caagaggtgg gaatgggaga aaaggagtga |
| 4381 | cgtgggagcg gagggtctgt atgtgtgcac ttgggcacgt atatgtgtgc tctgaaggtc |
| 4441 | aggattgcca gggcaaagta gcacagtctg gtatagtctg aagaagcggc tgctcagctg |
| 4501 | cagaagccct ctggtccggc aggatgggaa cggctgcctt gccttctgcc cacaccctag |
| 4561 | ggacatgagc tgtccttcca aacagagctc caggcactct cttggggaca gcatggcagg |
| 4621 | ctctgtgtgg tagcagtgcc tgggagttgg ccttttactc attgttgaaa taatttttgt |
| 4681 | ttattattta tttaacgata catatattta tatatttatc aatggggtat ctgcagggat |
| 4741 | gttttgacac catcttccag gatggagatt atttgtgaag acttcagtag aatcccagga |
| 4801 | ctaaacgtct aaattttttc tccaaacttg actgacttgg gaaaaccagg tgaatagaat |
| 4861 | aagagctgaa tgttttaagt aataaacgtt caaactgctc taagtaaaaa aatgcatttt |
| 4921 | actgcaatga atttctagaa tatttttccc ccaaagctat gcctcctaac ccttaaatgg |
| 4981 | tgaacaactg gtttcttgct acagctcact gccatttctt cttactatca tcactaggtt |
| 5041 | tcctaagatt cactcataca gtattatttg aagattcagc tttgttctgt gaatgtcatc |
| 5101 | ttaggattgt gtctatattc ttttgcttat ttctttttac tctgggcctc tcatactagt |
| 5161 | aagattttaa aaagcctttt cttctctgta tgtttggctc accaaggcga aatatatatt |
| 5221 | cttctctttt tcatttctca agaataaacc tcatctgctt ttttgttttt ctgtgttttg |
| 5281 | gcttggtact gaatgactca actgctcggt tttaaagttc aaagtgtaag tacttagggt |
| 5341 | tagtactgct tatttcaata atgttgacgg tgactatctt tggaaagcag taacatgctg |
| 5401 | tcttagaaat gacattaata atgggcttaa acaaatgaat aggggggtcc ccccactctc |
| 5461 | cttttgtatg cctatgtgtg tctgatttgt taaaagatgg acagggaatt gattgcagag |
| 5521 | tgtcgcttcc ttctaaagta gttttatttt gtctactgtt agtatttaaa gatcctggag |
| 5581 | gtggacataa ggaataaatg gaagagaaaa gtagatattg tatggtggct actaaaagga |
| 5641 | aattcaaaaa gtcttagaac ccgagcacct gagcaaactg cagtagtcaa aatatttatc |
| 5701 | tcatgttaaa gaaaggcaaa tctagtgtaa gaaatgagta ccatataggg ttttgaagtt |
| 5761 | catatactag aaacacttaa aagatatcat ttcagatatt acgtttggca ttgttcttaa |
| 5821 | gtatttatat ctttgagtca agctgataat taaaaaaaat ctgttaatgg agtgtatatt |
| 5881 | tcataatgta tcaaaatggt gtctatacct aaggtagcat tattgaagag agatatgttt |
| 5941 | atgtagtaag ttattaacat aatgagtaac aaataatgtt tccagaagaa aggaaaacac |
| 6001 | attttcagag tgcgttttta tcagaggaag acaaaaatac acacccctct ccagtagctt |
| 6061 | atttttacaa agccggccca gtgaattaga aaaacaaagc acttggatat gatttttgga |
| 6121 | aagcccaggt acacttatta ttcaaaatgc acttttactg agtttgaaaa gtttctttta |
| 6181 | tatttaaaat aagggttcaa atatgcatat tcaattttta tagtagttat ctatttgcaa |
| 6241 | agcatatatt aactagtaat tggctgttaa ttttatagac atggtagcca gggaagtata |
| 6301 | tcaatgacct attaagtatt ttgacaagca atttacatat ctgatgacct cgtatctctt |
| 6361 | tttcagcaag tcaaatgcta tgtaattgtt ccattgtgtg ttgtataaaa tgaatcaaca |
| 6421 | cggtaagaaa aaggttagag ttattaaaat aataaactga ctaaaatact catttgaatt |
| 6481 | tattcagaat gttcataatg ctttcaaagg acatagcaga gcttttgtgg agtatccgca |
| 6541 | caacattatt tattatctat ggactaaatc aattttttga agttgcttta aaatttaaaa |
| 6601 | gcacctttgc ttaatataaa gccctttaat tttaactgac agatcaattc tgaaacttta |
| 6661 | ttttgaaaag aaaatgggga agaatctgtg tctttagaat taaaagaaat gaaaaaaata |
| 6721 | aacccgacat tctaaaaaaa tagaataaga aacctgattt ttagtactaa tgaaatagcg |
| 6781 | ggtgacaaaa tagttgtctt tttgattttg atcacaaaaa ataaactggt agtgacagga |
| 6841 | tatgatggag agatttgaca tcctggcaaa tcactgtcat tgattcaatt attctaattc |
| 6901 | tgaataaaag ctgtatacag ta |

(SEQ ID NO: 2), (Homo sapiens paired box 6 (PAX6), transcript variant 4,
mRNA
NCBI Reference Sequence: NM_001258462.2), which encodes a polypeptide
with the amino acid sequence
MQNSHSGVNQLGGVFVNGRPLPDSTRQKIVELAHSGARPCDISRILQTHADAKVQVLDNQNVSNGCVSKIL
GRYYETGSIRPRAIGGSKPRVATPEVVSKIAQYKRECPSIFAWEIRDRLLSEGVCTNDNIPSVSSINRVLR
NLASEKQQMGADGMYDKLRMLNGQTGSWGTRPGWYPGTSVPGQPTQDGCQQQEGGGENTNSISSNGEDSDE
AQMRLQLKRKLQRNRTSFTQEQIEALEKEFERTHYPDVFARERLAAKIDLPEARIQVWFSNRRAKWRREEK
LRNQRRQASNTPSHIPISSSFSTSVYQPIPQPTTPVSSFTSGSMLGRTDTALTNTYSALPPMPSFTMANNL
PMQPPVPSQTSSYSCMLPTSPSVNGRSYDTYTPPHMQTHMNSQPMGTSGTTSTGLISPGVSVPVQVPGSEP
DMSQYWPRLQ
(SEQ ID NO: 9)

| | |
|---|---|
| 1 | gcacttagtc aacaaatggc acgtgggaga agttggtgag tgtttggtga ggactcttca |
| 61 | gggcttttca caagaaccct ctgtacacaa agaatctgag aattgctctc acacaccaac |
| 121 | ccagcaacat ccgtggagaa aactctcacc agcaactcct ttaaaacacc gtcatttcaa |
| 181 | accattgtgg tcttcaagca acaacagcag cacaaaaaac cccaaccaaa caaaactctt |
| 241 | gacagaagct gtgacaacca gaaaggatgc ctcataaagg gggaagactt taactagggg |
| 301 | cgcgcagatg tgtgaggcct tttattgtga gagtggacag acatccgaga tttcagagcc |
| 361 | ccatattcga gccccgtgga atcccgcggc ccccagccag agccagcatg cagaacagtc |
| 421 | acagcggagt gaatcagctc ggtggtgtct ttgtcaacgg gcggccactg ccggactcca |
| 481 | cccggcagaa gattgtagag ctagctcaca gcggggcccg gccgtgcgac atttcccgaa |
| 541 | ttctgcagac ccatgcagat gcaaaagtcc aagtgctgga caatcaaaac gtgtccaacg |
| 601 | gatgtgtgag taaaattctg ggcaggtatt acgagactgg ctccatcaga cccagggcaa |
| 661 | tcggtggtag taaaccgaga gtagcgactc cagaagttgt aagcaaaata gcccagtata |
| 721 | agcgggagtg cccgtccatc tttgcttggg aaatccgaga cagattactg tccgaggggg |
| 781 | tctgtaccaa cgataacata ccaagcgtgt catcaataaa cagagttctt cgcaacctgg |
| 841 | ctagcgaaaa gcaacagatg ggcgcagacg gcatgtatga taaactaagg atgttgaacg |
| 901 | ggcagaccgg aagctggggc acccgccctg gttggtatcc ggggacttcg gtgccagggc |
| 961 | aacctacgca agatggctgc cagcaacagg aaggaggggg agagaatacc aactccatca |
| 1021 | gttccaacgg agaagattca gatgaggctc aaatgcgact tcagctgaag cggaagctgc |
| 1081 | aaagaaatag aacatccttt acccaagagc aaattgaggc cctggagaaa gagtttgaga |
| 1141 | gaacccatta tccagatgtg tttgcccgag aaagactagc agccaaaata gatctacctg |
| 1201 | aagcaagaat acaggtatgg ttttctaatc gaagggccaa atggagaaga gaagaaaaac |
| 1261 | tgaggaatca gagaagacag gccagcaaca cacctagtca tattcctatc agcagtagtt |
| 1321 | tcagcaccag tgtctaccaa ccaattccac aacccaccac accggtttcc tccttcacat |
| 1381 | ctggctccat gttgggccga acagacacag ccctcacaaa cacctacagc gctctgccgc |
| 1441 | ctatgcccag cttcaccatg gcaaataacc tgcctatgca acccccagtc cccagccaga |
| 1501 | cctcctcata ctcctgcatg ctgcccacca gcccttcggt gaatgggcgg agttatgata |
| 1561 | cctacacccc cccacatatg cagacacaca tgaacagtca gccaatgggc acctcgggca |
| 1621 | ccacttcaac aggactcatt tcccctggtg tgtcagttcc agttcaagtt cccggaagtg |
| 1681 | aacctgatat gtctcaatac tggccaagat tacagtaaaa aaaaaaaaaa aaaaaaaaag |
| 1741 | gaaaggaaat attgtgttaa ttcagtcagt gactatgggg acacaacagt tgagctttca |
| 1801 | ggaaagaaag aaaaatggct gttagagccg cttcagttct acaattgtgt cctgtattgt |
| 1861 | accactgggg aaggaatgga cttgaaacaa ggacctttgt atacagaagg cacgatatca |
| 1921 | gttggaacaa atcttcattt tggtatccaa acttttattc attttggtgt attatttgta |
| 1981 | aatgggcatt tgtatgttat aatgaaaaaa agaacaatgt agactggatg gatgtttgat |
| 2041 | ctgtgttggt catgaagttg tttttttttt ttttaaaaag aaaaccatga tcaacaagct |
| 2101 | ttgccacgaa tttaagagtt ttatcaagat atatcgaata cttctaccca tctgttcata |
| 2161 | gtttatggac tgatgttcca agtttgtatc attcctttgc atataattaa acctggaaca |
| 2221 | acatgcacta gatttatgtc agaaatatct gttggttttc caaaggttgt taacagatga |
| 2281 | agtttatgtg caaaaaaggg taagatataa attcaaggaa gaaaaaaagt tgatagctaa |
| 2341 | aaggtagagt gtgtcttcga tataatccaa tttgttttat gtcaaaatgt aagtatttgt |
| 2401 | cttccctaga aatcctcaga atgatttcta taataaagtt aatttcattt atatttgaca |
| 2461 | agaatataga tgttttatac acattttcat gcaatcatac gtttcttttt tggccagcaa |
| 2521 | aagttaattg ttcttagata tagttgtatt actgttcacg gtccaatcat tttgtgcatc |
| 2581 | tagagttcat tcctaatcaa ttaaaagtgc ttgcaagagt tttaaactta agtgttttga |
| 2641 | agttgttcac aactacatat caaaattaac cattgttgat tgtaaaaaac catgccaaag |
| 2701 | cctttgtatt tcctttatta tacagttttc tttttaacct tatagtgtgg tgttacaaat |
| 2761 | tttatttcca tgttagatca acattctaaa ccaatggtta ctttcacaca cactctgttt |
| 2821 | tacatcctga tgatccttaa aaaataatcc ttatagatac cataaatcaa aaacgtgtta |
| 2881 | gaaaaaaatt ccacttacag cagggtgtag atctgtgccc atttataccc acaacatata |
| 2941 | tacaaaatgg taacatttcc cagttagcca tttaattcta aagctcaaag tctagaaata |
| 3001 | atttaaaaat gcaacaagcg attagctagg aattgttttt tgaattagga ctggcatttt |
| 3061 | caatctgggc agatttccat tgtcagccta tttcaacaat gatttcactg aagtatattc |
| 3121 | aaaagtagat ttcttaaagg agactttctg aaagctgttg cctttttcaa ataggccctc |
| 3181 | tcccttttct gtctccctcc cctttgcaca agaggcatca tttcccattg aaccactaca |
| 3241 | gctgttccca tttgaatctt gctttctgtg cggttgtgga tggttggagg gtggaggggg |
| 3301 | gatgttgcat gtcaaggaat aatgagcaca gacacatcaa cagacaacaa caaagcagac |
| 3361 | tgtgactggc cggtgggaat taaaggcctt cagtcattgg cagcttaagc caaacattcc |
| 3421 | caaatctatg aagcagggcc cattgttggt cagttgttat ttgcaatgaa gcacagttct |
| 3481 | gatcatgttt aaagtggagg cacgcagggc aggagtgctt gagcccaagc aaaggatgga |
| 3541 | aaaaaataag cctttgttgg gtaaaaaagg actgtctgag actttcattt gttctgtgca |
| 3601 | acatataagt caatacagat aagtcttcct ctgcaaactt cactaaaaag cctgggggtt |
| 3661 | ctggcagtct agattaaaat gcttgcacat gcagaaacct ctggggacaa agacacactt |
| 3721 | ccactgaatt atactctgct ttaaaaaaat ccccaaaagc aaatgatcag aaatgtagaa |
| 3781 | attaatggaa ggatttaaac atgaccttct cgttcaatat ctactgtttt ttagttaagg |
| 3841 | aattacttgt gaacagataa ttgagattca ttgctccggc atgaaatata ctaataattt |
| 3901 | tattccacca gagttgctgc acatttggag acaccttcct aagttgcagt ttttgtatgt |
| 3961 | gtgcatgtag ttttgttcag tgtcagcctg cactgcacag cagcacattt ctgcagggga |
| 4021 | gtgagcacac atacgcactg ttggtacaat tgccggtgca gacatttcta cctcctgaca |
| 4081 | ttttgcagcc tacattccct gagggctgtg tgctgaggga actgtcagag aagggctatg |
| 4141 | tgggagtgca tgccacagct gctggctggc ttacttcttc cttctcgctg gctgtaattt |
| 4201 | ccaccacggt caggcagcca gttccggccc acggttctgt tgtgtagaca gcagagactt |
| 4261 | tggagacccg gatgtcgcac gccaggtgca agaggtggga atgggagaaa aggagtgacg |
| 4321 | tgggagcgga gggtctgtat gtgtgcactt gggcacgtat atgtgtgctc tgaaggtcag |
| 4381 | gattgccagg gcaaagtagc acagtctggt atagtctgaa gaagcggctg ctcagctgca |
| 4441 | gaagccctct ggtccggcag gatgggaacg gctgccttgc cttctgccca caccctaggg |
| 4501 | acatgagctg tccttccaaa cagagctcca ggcactctct tggggacagc atggcaggct |
| 4561 | ctgtgtggta gcagtgcctg ggagttggcc ttttactcat tgttgaaata atttttgttt |
| 4621 | attatttatt taacgataca tatatttata tatttatcaa tggggtatct gcagggatgt |
| 4681 | tttgacacca tcttccagga tggagattat ttgtgaagac ttcagtagaa tcccaggact |
| 4741 | aaacgtctaa attttttctc caaacttgac tgacttggga aaaccaggtg aatagaataa |
| 4801 | gagctgaatg ttttaagtaa taaacgttca aactgctcta agtaaaaaaa tgcattttac |
| 4861 | tgcaatgaat ttctagaata tttttccccc aaagctatgc ctcctaaccc ttaaatggtg |
| 4921 | aacaactggt ttcttgctac agctcactgc catttcttct tactatcatc actaggtttc |
| 4981 | ctaagattca ctcatacagt attatttgaa gattcagctt tgttctgtga atgtcatctt |
| 5041 | aggattgtgt ctatattctt ttgcttattt ctttttactc tgggcctctc atactagtaa |
| 5101 | gattttaaaa agccttttct tctctgtatg tttggctcac caaggcgaaa tatatattct |
| 5161 | tctctttttc atttctcaag aataaacctc atctgctttt ttgtttttct gtgttttggc |
| 5221 | ttggtactga atgactcaac tgctcggttt taaagttcaa agtgtaagta cttagggtta |
| 5281 | gtactgctta tttcaataat gttgacggtg actatctttg gaaagcagta acatgctgtc |
| 5341 | ttagaaatga cattaataat gggcttaaac aaatgaatag gggggtcccc ccactctcct |
| 5401 | tttgtatgcc tatgtgtgtc tgatttgtta aaagatggac agggaattga ttgcagagtg |
| 5461 | tcgcttcctt ctaaagtagt tttattttgt ctactgttag tatttaaaga tcctggaggt |
| 5521 | ggacataagg aataaatgga agagaaaagt agatattgta tggtggctac taaaaggaaa |
| 5581 | ttcaaaaagt cttagaaccc gagcacctga gcaaactgca gtagtcaaaa tatttatctc |
| 5641 | atgttaaaga aaggcaaatc tagtgtaaga aatgagtacc atatagggtt ttgaagttca |
| 5701 | tatactagaa acacttaaaa gatatcattt cagatattac gtttggcatt gttcttaagt |
| 5761 | atttatatct ttgagtcaag ctgataatta aaaaaaatct gttaatggag tgtatatttc |
| 5821 | ataatgtatc aaaatggtgt ctatacctaa ggtagcatta ttgaagagag atatgtttat |
| 5881 | gtagtaagtt attaacataa tgagtaacaa ataatgtttc cagaagaaag gaaaacacat |
| 5941 | tttcagagtg cgtttttatc agaggaagac aaaaatacac acccctctcc agtagcttat |
| 6001 | ttttacaaag ccggcccagt gaattagaaa aacaaagcac ttggatatga tttttggaaa |
| 6061 | gcccaggtac acttattatt caaaatgcac ttttactgag tttgaaaagt ttcttttata |
| 6121 | tttaaaataa gggttcaaat atgcatattc aatttttata gtagttatct atttgcaaag |
| 6181 | catatattaa ctagtaattg gctgttaatt ttatagacat ggtagccagg gaagtatatc |
| 6241 | aatgacctat taagtatttt gacaagcaat ttacatatct gatgacctcg tatctctttt |
| 6301 | tcagcaagtc aaatgctatg taattgttcc attgtgtgtt gtataaaatg aatcaacacg |
| 6361 | gtaagaaaaa ggttagagtt attaaaataa taaactgact aaaatactca tttgaattta |
| 6421 | ttcagaatgt tcataatgct ttcaaaggac atagcagagc ttttgtggag tatccgcaca |
| 6481 | acattattta ttatctatgg actaaatcaa ttttttgaag ttgctttaaa atttaaaagc |
| 6541 | acctttgctt aatataaagc cctttaattt taactgacag atcaattctg aaactttatt |
| 6601 | ttgaaaagaa aatggggaag aatctgtgtc tttagaatta aaagaaatga aaaaaataaa |
| 6661 | cccgacattc taaaaaaata gaataagaaa cctgattttt agtactaatg aaatagcggg |
| 6721 | tgacaaaata gttgtctttt tgattttgat cacaaaaaat aaactggtag tgacaggata |
| 6781 | tgatggagag atttgacatc ctggcaaatc actgtcattg attcaattat tctaattctg |
| 6841 | aataaaagct gtatacagta aaa |

(SEQ ID NO: 3) (Homo sapiens paired box 6 (PAX6), transcript variant 5,
mRNA
NCBI Reference Sequence: NM_001258463.1), which encodes a polypeptide
with the amino acid sequence
MQNSHSGVNQLGGVFVNGRPLPDSTRQKIVELAHSGARPCDISRILQTHADAKVQVLDNQNVSNGCVSKIL
GRYYETGSIRPRAIGGSKPRVATPEVVSKIAQYKRECPSIFAWEIRDRLLSEGVCTNDNIPSVSSINRVLR
NLASEKQQMGADGMYDKLRMLNGQTGSWGTRPGWYPGTSVPGQPTQDGCQQQEGGGENTNSISSNGEDSDE
AQMRLQLKRKLQRNRTSFTQEQIEALEKEFERTHYPDVFARERLAAKIDLPEARIQVWFSNRRAKWRREEK
LRNQRRQASNTPSHIPISSSFSTSVYQPIPQPTTPVSSFTSGSMLGRTDTALTNTYSALPPMPSFTMANNL
PMQPPVPSQTSSYSCMLPTSPSVNGRSYDTYTPPHMQTHMNSQPMGTSGTTSTGLISPGVSVPVQVPGSEP
DMSQYWPRLQ
(SEQ ID NO: 10)

| | |
|---|---|
| 1 | gcatgttgcg gagtgattag tgggtttgaa aagggaaccg tggctcggcc tcatttcccg |
| 61 | ctctggttca ggcgcaggag gaagtgtttt gctggaggat gatgacagag gaatctgaga |
| 121 | attgctctca cacaccaacc cagcaacatc cgtggagaaa actctcacca gcaactcctt |
| 181 | taaaacaccg tcatttcaaa ccattgtggt cttcaagcaa caacagcagc acaaaaaacc |
| 241 | ccaaccaaac aaaactcttg acagaagctg tgacaaccag aaaggatgcc tcataaaggg |
| 301 | ggaagacttt aactaggggc gcgcagatgt gtgaggcctt ttattgtgag agtggacaga |
| 361 | catccgagat ttcagagccc catattcgag ccccgtggaa tcccgcggcc cccagccaga |
| 421 | gccagcatgc agaacagtca cagcggagtg aatcagctcg gtggtgtctt tgtcaacggg |
| 481 | cggccactgc cggactccac ccggcagaag attgtagagc tagctcacag cggggcccgg |
| 541 | ccgtgcgaca tttcccgaat tctgcaggtg tccaacggat gtgtgagtaa aattctgggc |
| 601 | aggtattacg agactggctc catcagaccc agggcaatcg gtggtagtaa accgagagta |
| 661 | gcgactccag aagttgtaag caaaatagcc cagtataagc gggagtgccc gtccatcttt |
| 721 | gcttgggaaa tccgagacag attactgtcc gagggggtct gtaccaacga taacatacca |
| 781 | agcgtgtcat caataaacag agttcttcgc aacctggcta gcgaaaagca acagatgggc |
| 841 | gcagacggca tgtatgataa actaaggatg ttgaacgggc agaccggaag ctggggcacc |
| 901 | cgccctggtt ggtatccggg gacttcggtg ccagggcaac ctacgcaaga tggctgccag |
| 961 | caacaggaag gagggggaga gaataccaac tccatcagtt ccaacggaga agattcagat |
| 1021 | gaggctcaaa tgcgacttca gctgaagcgg aagctgcaaa gaaatagaac atcctttacc |
| 1081 | caagagcaaa ttgaggccct ggagaaagag tttgagagaa cccattatcc agatgtgttt |
| 1141 | gcccgagaaa gactagcagc caaaatagat ctacctgaag caagaataca ggtatggttt |
| 1201 | tctaatcgaa gggccaaatg gagaagagaa gaaaaactga ggaatcagag aagacaggcc |
| 1261 | agcaacacac ctagtcatat tcctatcagc agtagtttca gcaccagtgt ctaccaacca |
| 1321 | attccacaac ccaccacacc ggtttcctcc ttcacatctg gctccatgtt gggccgaaca |
| 1381 | gacacagccc tcacaaacac ctacagcgct ctgccgccta tgcccagctt caccatggca |
| 1441 | aataacctgc ctatgcaacc cccagtcccc agccagacct cctcatactc ctgcatgctg |
| 1501 | cccaccagcc cttcggtgaa tgggcggagt tatgatacct acaccccccc acatatgcag |
| 1561 | acacacatga acagtcagcc aatgggcacc tcgggcacca cttcaacagg actcatttcc |
| 1621 | cctggtgtgt cagttccagt tcaagttccc ggaagtgaac ctgatatgtc tcaatactgg |
| 1681 | ccaagattac agtaaaaaaa aaaaaaaaaa aaaaaaggaa aggaaatatt gtgttaattc |
| 1741 | agtcagtgac tatggggaca caacagttga gctttcagga aagaaagaaa aatggctgtt |
| 1801 | agagccgctt cagttctaca attgtgtcct gtattgtacc actggggaag gaatggactt |
| 1861 | gaaacaagga cctttgtata cagaaggcac gatatcagtt ggaacaaatc ttcattttgg |
| 1921 | tatccaaact tttattcatt ttggtgtatt atttgtaaat gggcatttgt atgttataat |
| 1981 | gaaaaaaaga acaatgtaga ctggatggat gtttgatctg tgttggtcat gaagttgttt |
| 2041 | tttttttttt taaaaagaaa accatgatca acaagctttg ccacgaattt aagagtttta |
| 2101 | tcaagatata tcgaatactt ctacccatct gttcatagtt tatggactga tgttccaagt |
| 2161 | ttgtatcatt cctttgcata taattaaacc tggaacaaca tgcactagat ttatgtcaga |
| 2221 | aatatctgtt ggttttccaa aggttgttaa cagatgaagt ttatgtgcaa aaaagggtaa |
| 2281 | gatataaatt caaggaagaa aaaaagttga tagctaaaag gtagagtgtg tcttcgatat |
| 2341 | aatccaattt gttttatgtc aaaatgtaag tatttgtctt ccctagaaat cctcagaatg |
| 2401 | atttctataa taaagttaat ttcatttata tttgacaaga atatagatgt tttatacaca |
| 2461 | ttttcatgca atcatacgtt tcttttttgg ccagcaaaag ttaattgttc ttagatatag |
| 2521 | ttgtattact gttcacggtc caatcatttt gtgcatctag agttcattcc taatcaatta |
| 2581 | aaagtgcttg caagagtttt aaa |

(SEQ ID NO: 4) (Homo sapiens paired box 6 (PAX6), transcript variant 6,
mRNA
NCBI Reference Sequence: NM_001258464.2, which encodes a polypeptide
with the amino acid sequence
MQNSHSGVNQLGGVFVNGRPLPDSTRQKIVELAHSGARPCDISRILQVSNGCVSKILGRYYETGSIRPRAI
GGSKPRVATPEVVSKIAQYKRECPSIFAWEIRDRLLSEGVCTNDNIPSVSSINRVLRNLASEKQQMGADGM
YDKLRMLNGQTGSWGTRPGWYPGTSVPGQPTQDGCQQQEGGGENTNSISSNGEDSDEAQMRLQLKRKLORN
RTSFTQEQIEALEKEFERTHYPDVFARERLAAKIDLPEARIQVWFSNRRAKWRREEKLRNORRQASNTPSH
IPISSSFSTSVYQPIPQPTTPVSSFTSGSMLGRTDTALTNTYSALPPMPSFTMANNLPMQPPVPSQTSSYS
CMLPTSPSVNGRSYDTYTPPHMQTHMNSQPMGTSGTTSTGLISPGVSVPVQVPGSEPDMSQYWPRLQ
(SEQ ID NO: 11)

| | |
|---|---|
| 1 | atcgatttcg ggcagtcgat aaaacaacag ttcggagtgc agcaaaagtg cgggaaatct |
| 61 | gcggaaatgt tattaatcga acattaaggg atacctcgcc cagccgcgct ctagcattca |
| 121 | ccatttccct ttgtttgcgg cggcggcgct cgttgtacgg ccgtgcgaat cgagagaaaa |
| 181 | acgcataaaa tcggggaaag tgcactagcc ccgtgctgca atgtaaataa acaaggctct |
| 241 | gatgagcagt tgccgatcaa gtgcttcgat tagccagcaa aatgtgcgta caaggcgaga |
| 301 | atcagtgacg aaaacaacaa ctacgtgctg ctgcgttcat tgacgtgacg cgcgagagtg |
| 361 | aatgtgtgcg tgcgtgtgtg tttgtgtgtg tgtgctggct gctggcgagt gagtgtgtgc |
| 421 | gtgctcccca tatgtgtgtt gtcgaaaaag cgtttttcaa accacaagcc agcaacaaac |
| 481 | aaatcacaac agcacgtcac aaattgcaat agaagcagaa gcagcaacac ctcaaatcaa |
| 541 | aaaaaggccc cacaacaaca acatctgtcg agttctcaga gagtgagaaa aagaaagagc |
| 601 | gcgttagagt gagagagggg caaaaagaaa gtcaacagtc gcgtgtgaaa tgaaattcga |
| 661 | aagatgtccg cgtgtgtgtg tgttgagtgt atagaattga gtaagaagca gcgcggcaaa |
| 721 | aggaaataag caaaagcaat agccaaaagg aaccaacaac cagcagcaac aaattaccca |
| 781 | atgaatcaat caaaattggc ataatttgca ttcgccgacg ttattgtcta taaaaccccc |
| 841 | ctgaaaaaaa caccacacaa cagaagagca gcacaaatct tttgccaatg agtcgcagac |
| 901 | agtcattgga tcgaccagga atattgtctc ctccgggtcc gtttctgacg cccagtccat |
| 961 | cgccatcgat ctcagctagc cctcgtctgc aaccatcgcc atcgctctcg ccgttcccac |
| 1021 | aggatgtggt gctcgtagcc ggtggcagcc aagtaaacgc caacagcaat ccccaggaac |
| 1081 | acgagcgcaa ggcggcgacg cctggcagga gacagtccat ccaccagttc accaatggca |
| 1141 | gtcccaatgc tggaactgtc gtcttccagc cgagcccctc acaatcgccg gccgctgcca |
| 1201 | cgacggtcat cggcgtgacc agtggtggcc tcatagccac cgccgccgga ggaactggag |
| 1261 | ccggtctgag taccggagta ggagccggga gcagttccgg agtaggaatt ggcgtcgcgc |
| 1321 | tgcacggaaa tgccatcggc aacgagctga atgccactct ggtgggatcg aataatatcc |
| 1381 | agctcaataa gaagggcaca aagcgggcaa cccagcagca gcagcaacaa cagcaacagc |
| 1441 | tgggacacca gatcttcagc agcacagttg cacccacctc gatcagcagt gcgactgcgg |
| 1501 | tggcaggcgg cggcgggggc tatggacagt tcaatgccac cgccatcagc acgcccacct |
| 1561 | cgttcgccac ggccggcagt gtggtcagca gcagcgccaa aggtccgacg aagggtcaga |
| 1621 | aggggcagca gcagcagcag cagcatcagc agttccagca ctaccagagc agcagtcccc |
| 1681 | tgatagcgga cagtcccatc ccgagtccca gcggcgccat tgcagcggcg gcgattaagc |
| 1741 | ctccaacccc gcagccccaa caaatgcaga tgctgcccca gcagttcacc atctcccagc |
| 1801 | agccgcagca gcagcagcag gcgcagcagg tcttccagtt tatccaggga ccgcagggcc |
| 1861 | agctcatagc caccactccc cagcaacagc agcctatcca gcagcagcaa cagcagcagc |
| 1921 | ctcagccagt ccagcagcag cagcagcatc gattcgttag caatgccgga gtcacgggaa |
| 1981 | tgtccaccgg caagaccggc aaggcaccgc agcagatact gcccaagccg caacaggagc |
| 2041 | tccagcagca gaagaagggc acgaacggcg gagcacagat ctcgcaatcg gcgcagcaaa |
| 2101 | ctggccaagc caacaatccc acccaacagc agcaacaaca acaacagcaa cagatcctgc |
| 2161 | tacctgccac caccaaccag ccgcaacagt tgctccttaa ccaaatgccc gtgctggtgc |
| 2221 | aacagaatcc gcagggcgtg cagctaattc tgcgaccacc tacgcctcag ctgacgacca |
| 2281 | caccgtccct cgtcatccag cagaatgccc gcgggcaacc ccaactgcag acgcagccgg |
| 2341 | cacagcaaca actgctgcgg atagtgggca ccaatggagc gacaatgcag ctggctgccg |
| 2401 | cgcccacctt cattgtgtcc tcgcaggcga acctcatcca tcagacggcg gcaggtcagt |
| 2461 | tccagagcgc catcaagtct ggcacccagc tcacgggtct gcatgcggct ctagcacagg |
| 2521 | cacaggccca ggcgcaggcg ccacgatctc agccgtttgc cacgacggcc acgttgaaca |
| 2581 | cccagctcct tggccagagt gtagccgccc agctgcagaa cttacagctg gctgcggccc |
| 2641 | aaattcagat gcccaacggc ctcaccgcca tatcccaact gccagctcat ttgcagcaga |
| 2701 | gcctaggtgg agccaccatc aatctgaatc agctgaatgg ggcgcatatc cagcaaatag |
| 2761 | ctaacgcttt tcaggcgcca caggctccag gaacgaatag cggaggagct aacagcacga |
| 2821 | cggagctctt tacacaatcc tcgccggcac atgtgccgct ggccgtcacc ccggagccat |
| 2881 | tgcgtcagcc cacgcccgtg cccatgatgc agcaacaaca acaggctgcg atggccacca |
| 2941 | ttcagttgca gcagcagcag caacaacagc agcaggcgca gatacagcag ctgcagcatc |
| 3001 | aactgcagca aacacagtcc caggtgcagc aagcccagca atctgtccag gcgacaacta |
| 3061 | taccggagcc caagaagaaa ccgcgacccc gcaaaaagaa gcaacctcca gctccaactc |
| 3121 | caccagcagc ctctccgaca gtagcacccg ctgaagtgag acccccgacg ccacaaaaga |
| 3181 | tagccatcgc cgccacgccc gcgccacagc caactgtacg cgccaagtct ccatcgccac |
| 3241 | ccccaacgac gatccagcga agtagcaatg gaaagctgga tctcggcgat gtgatgaagt |
| 3301 | tttgcggtat caccggagac gatgacgacg acgaggacta tggaatgggc ctagaaacag |
| 3361 | ggctagcatc agaatcagag ccagctcaag cccacgcgcc tgccacaggg agtgctgcag |
| 3421 | cgacgacggg cagcagcggc gacatcatga tctccatacc gaaccaaaat ggcagcgatg |
| 3481 | gcctgccttt taccttgacc atacccgctc cgcagcccca gtcatcgacg ggatctcagg |
| 3541 | cggcgggaaa cgaatcagcc accgaaatac caaatatcct catcaaaatc gatccaagtg |
| 3601 | ccgaggcagc ggtgccgcca tttggtctat ccacgccacg gttgcccaca gcagaggaaa |
| 3661 | ttcagaagca ggcgcagcaa catttggcac aacaggcggc cgctgcagca gcggcggcaa |
| 3721 | aggcaaccac agccacgccc accataaaca ttagcctgcc tagcatgaca ttgcagccgc |
| 3781 | aggtagcgcc ggcaccagtt gtgacgccaa ctcctgcccc atcctcgaac caggtgtcca |
| 3841 | cgacagttgt tgtcaaacca agaaggaagc ccgcggtgcg aaggaatgcc aagaaacctg |
| 3901 | acgtgaccag cagcgcaagc aatatgatct cctcgagcag tggcactacc actacaatca |
| 3961 | gcagcagcac tagtaccatc ctaaacaaca gtcaacccac cactgtaaac accatcactt |
| 4021 | taaccacgcc cacgccagtg acaagcagca gcaattccac cctgatgctg acccacggaa |
| 4081 | ctccgacagc agtgcccagt cagattggca acattcagat ctcacaggtg cataatcatc |
| 4141 | acccatccgc attgccggtg agcagtgcgg tggagaacaa gatccagata atgccgatcc |
| 4201 | tgccaccggg agccacagtg atgggcggaa caccaggaac cggaggtcat gcccccacca |
| 4261 | cccaatcaac ccagttggtc tttcagccag cggcaccatc ggtggcgcct acgattactt |
| 4321 | cagactccac tgggtttaag ctctccagcg atggtcggca aatgcagcta gtggccatac |
| 4381 | cacaggcgac tcctccatcg ccagcagcca gttctgccca gccaccggtt cccacgccca |
| 4441 | ctcctgcgtt aactgccgga ggagctacaa ttctgccacc tcagttgaca ggaatgcccg |
| 4501 | ccaatgtatc ggtttctgtg ggatcccctg cctcggctgc ggctctggtg ccacaactca |
| 4561 | ctggaagtct cacactcacc gtgtcggagc agtgcgagcg attgattctg cgacatgatc |
| 4621 | cgaacaatcc ccaggaccat caatcgcagc tcatcctgca ggctctgctc aagggcgctc |
| 4681 | tgcccaatgt taccataatc aatgagccga cgcgcgtgga tccaaataaa cagacaactc |
| 4741 | cgatgttgca gccgcaacag caagttatcc aaatacccca gccactaact caaccccagc |
| 4801 | aaattctgca gcagcaacca aaggtttcaa cagctcccaa aatagaagtt aaagtggcta |
| 4861 | acaacagaaa gttgtcaggt caggtaacac atcctgccag cagtatgtta ggaatgacca |
| 4921 | gcactacaac taccatgtca acgatgaaac caccggccaa tctacccgcc aagcagaacg |
| 4981 | aagtcgtatc tttcccgcag ctaccactga actcgcaggt tctcattcag cagcagccta |
| 5041 | tgatttctgc gcagctacag cctcagttgc aacccgtcac tgtgcccgtt agcttgccca |
| 5101 | ctcagccggt accgattcca tctccggcgc ctgctccaca actggccaac aatggccagc |
| 5161 | agcggtacat tgcactgccg cccattgatc ccaccaccca gcagttgttc tgcctgaaca |
| 5221 | gtgtcacgaa tcagataact gcgatgagtg cgggccagac ggctgcttcg attggtccca |
| 5281 | ccgagcggtt gcttattgct ccggctggta tcaatgctca gcagctggct caatgtctgc |
| 5341 | agttggggca acttcatttc aatgatgtga atccgctgcc ggctcagcag caacagcagc |
| 5401 | aggtggctac cacctcgtct tcgatgacac tttcaatgcc actgcgtcct cagccaccgc |
| 5461 | agcaacaaat agtgcatccc atccagccaa tcacaaatgg actggccatc accacaacgg |
| 5521 | caagtagcac gctgaccacc accactagca ccacttcgtt aacaacggtg accaagcagg |
| 5581 | tggccaatgt ggcgccgatt atagatcaaa gcaaagcgaa gctggagccc gctaaggcag |
| 5641 | ccaccagtgg agccggtggc ggagcggtgg tcaaaaagaa gccggtgagg aagcccaagg |
| 5701 | caacgccgac caatgcctcc gaactggcca atctaaagaa cgccagcgta gtcaagccaa |
| 5761 | tgccaaagct cgatccccta tcgcaaaagc ccaacaataa cccggtgcag atcgtgcagc |
| 5821 | ctaatgttgc cagtggaaag cagatgattg tggtgggcag ctccagttgc acaacaacta |
| 5881 | ccacgacaac gatgagcgcc agcatccagc ccttccagac aacgagtggc accaaattgg |
| 5941 | tgggcgttac ggtgcccatg cagcagcaac aacccactgg cacggcagct atattgcaac |
| 6001 | agcagcagca acagaggacg agcttcagct tgacggcaac cacagtggcg acacctgctc |
| 6061 | cgatgccatc tccaacggta gcgtctgggg tcagccaact ccaaatgcag caacttccat |
| 6121 | cccagcaggc gcagcaacag ttgcagccgc agcagcagca acagttgcac gctcctaagc |
| 6181 | aacaacaacc gcaaccaaat actgtgtccc gggtgcaaac catccaactt acgcctcaga |
| 6241 | tgcaacagat cttcaagcag gtccaaatgc aaattcagtt cctcactatt aagttacaaa |
| 6301 | ataaatcaac cttcttgccc gtctcgccgg aaattgattc ggcaactatt gccgcctaca |
| 6361 | acaaaccgat gacagatgcg gagataaatc tggctctgca gcgtctcttc gcagagcagc |
| 6421 | accggattct ggcctccggc aaagaggtac ccactccaga gggtatgttg ccaacagcaa |
| 6481 | atggaacggg cggcttcagt ctcatgccac aaccagttca acaacaacaa cagcagcagc |
| 6541 | agcaacagtt gcaatcaact gttcctgagc cactgaaaac aatcgttccg gccaatgtgg |
| 6601 | ttcagccaaa caaccactca tccactcctg caggagcggc cactagtggt gccacaactg |
| 6661 | ctgccaatat ccagcagaat ataacacaga ggatacacat ctaccctatg cagcaccaac |
| 6721 | aacaacaaca gcagcaacaa cagcagcagc aacaacagcc gcagcatcag caacaacagc |
| 6781 | cccagcatca gctacaacag accggagcca aaccaaagca ggcacaacca aagccgccgc |
| 6841 | cacagcagga gcaacaatcg caactccaaa taaaacccaa ggaaccggcc aacaggagca |
| 6901 | aagtgactag ccagcagctg cagcaaaatc ctcagccacc caatggaacc atcaatatgt |
| 6961 | tgccacaaaa agttagcatg atgccggttg tccagcagca acagcagcag cagccgcaac |
| 7021 | aacatcagca gcaaccaatg ctcaacaaaa gcgggccacc tccactgatc ttcgccagct |
| 7081 | ctacaaatat gctgagcaac agcaatagca acaacctcca caacagcaat tctgcaatgc |
| 7141 | aagtaaacaa catgttgccc ctgccaccat tgcaacccca acagcagcct cagccacagt |
| 7201 | tacagcagcc accaactcca atcctgccgc cagtagcacc aacaatggcc atgggagtgg |
| 7261 | cgccaggacc gatgggaaat ctagtgccat ccctcccgac cataccgagc ctgcccctgt |
| 7321 | atatgccgag caaaatggac gaaatgccaa cacagaaacc aaaaattgca cgcctctcct |
| 7381 | tgttcgttcg ccaactggag gtcgatcagg agagctgtct gaagccggat tacgtgaagc |
| 7441 | ctttccgcac aaaggatgag gcggttaaaa ggctaatcag gtatcattgc atgcatgaga |
| 7501 | acgacgttga gttgccttct gacgaagacg aggaatttga gtccactgct ctggaattcc |
| 7561 | aggacaaatt ccgccaactg aacggcaagt tccaggaaat tctgatgcag gagtcaacgc |
| 7621 | tgccacaccg aacttctgag ttgctacaaa tgcagcagct gatgatcgat gatctcaaag |
| 7681 | gcgagatcaa tgagattcga accgctgaaa aggagttaga gcagcagcta aaggaggagc |
| 7741 | agactagcga aaaatcgacg gcggaaagcg atgtactttc ggaggcgaaa gttaaggagg |
| 7801 | agattaaaca ggaaccaata gataagtctg ctcagccaga cggcgttgtt gacaaatttg |
| 7861 | atttgctgaa aaacagcgct gatgtcaata aagcattcag caagtcacag ccacagcaga |
| 7921 | ctactacagt aaagcaagag gagagcaacg aatctgggga accttctgta aatggatctg |
| 7981 | taaagagcga aggccacgat aaggagtcgt cgaaatacat taagaaggac tacgacaact |
| 8041 | tcgacattga gtctgagctc accactagct tcatcatgaa gaaggtggag aacaaagcgg |
| 8101 | ctttggcaaa gagcgctgag aatcgctacc aggaaagaaa tatgatggac caacaacagc |
| 8161 | agcagcaaca tatcaaagcc aatggaggag atcccgtaga ccaggatgat ggctggtact |
| 8221 | gcctgcaaaa ggagcttaac ctgatgaata acgatcatat gcagcaatcg cagcaacaac |
| 8281 | taaatggaaa ccaacagcat ctgcccatca atcgtcaacc ggcgccacag caatcacact |
| 8341 | tcctggacaa ttcaaattcg aacaacatga tgggcaatag caaccacatg tcggatctgt |
| 8401 | tccccaatgg ttgcattgac aagagcaatc aaagcaccac cagcagcagc aatagcagtt |
| 8461 | gcgttagcga aagccttcct ggttctaatc ccgtgaagac tgtctcctca tgcagcggcc |
| 8521 | agcgcgaaca gccggtggtc atggatgctg atcagcaaaa cgagcatccc gccatcagtg |
| 8581 | agttctttag cagcgattcg gacgtccaga aatccgtgga gacacgactc gaggcaatgt |
| 8641 | tcggggagtc gcccgtccat ttggacgtta agagcagcaa cgatgccaac gacattgagt |
| 8701 | ccaacctgga cgagattttt ggcgactcga aatcgccagc tgccaaccac aaaagtaaaa |
| 8761 | tgagcatgtg ggtgccagac gcatcattta tgcagcaggc gccgcagcaa cagacgtcgc |
| 8821 | aacagcagca acaatacgca gggccacaac aaccatcaca gcagcaacat cacatcgaac |
| 8881 | tgaactgcaa caacaatccg cgctggatgc aaagcatgga ggcgcactac agcgactttc |
| 8941 | tttccaccgg gactagcaat ggagagctgg tgaacggaga atcgcgaaaa agaagctggg |
| 9001 | atagccagct catgggctct ggcagtgata tggaagatga caacagcagc aagcgtctgt |
| 9061 | gcacggcttc atcgtcctcc tcttcgtcgc ccatgtcgtc gcagcagcag cacgttcagg |
| 9121 | tggcacagca tggtctcttg gactcggaca tgttccccca gatgttgatg gatcagcagc |
| 9181 | cgcatcagca acaacagcag gtacagcagc aacacaactt tgcctacgcc aacataatgg |
| 9241 | atcaacagca gcagcaacag caccagcagc aacatttcca gcataacctg cagcaacaga |
| 9301 | tacaacagca gcagcagatg catgttagcc acatgggcgg agcagcagta gtggccggcg |
| 9361 | gagagtacga cgatgacatc agtcggcatg tggccagcgc catcgacagc atcctgaact |
| 9421 | tgcagaacag cggcgactcg ctgcagttct ccctgggttc gatcctcggt gacagcatgc |
| 9481 | tggacgatca acggcaagca ggagccaatt tgcccagctg ccagcaggag caggcgatcc |
| 9541 | aacgacgccg ccacctgggc gaggagatga acgactgcct gattagcggc ggtggttccg |
| 9601 | cggttagcgg agtggcggac aacagtagta acatactgct agagcaccac caccagcagc |
| 9661 | atcagttgat gcagagccac caacagcagc cgcatctgca gcatcaccac catcagaaca |
| 9721 | tgtcgcaggc ccaggcgcag cacatgggac agctgaacga ctttagctgt gtagccggtg |
| 9781 | gcttggacga tcctgtcaag tcgataatga cgtcctgact tggagcccca agcagcacat |
| 9841 | cagcggcgga aaatgcttcg aacagtctga tactagagtt taatgccacc accttgtgaa |
| 9901 | aaacggatga taataatgac ttcctgattg tatagatacc cgtcttagtt gtccttggtt |
| 9961 | attgttttac ccccgagtac aacctttaat tagatctaag ttgaatccgt gtagcataaa |
| 10021 | ttgtttttac ttattgtttg tattgtttag ttcaatgatt tgtaccatat ttgaacagcc |
| 10081 | ctcagataac caattactta atgcaagggg acagaacgaa gagagcgagg tgaaagtaat |
| 10141 | ggcatctaca tacaatacgg ctgagcccca ataaagctac gaaagcaatt gacgtggtga |
| 10201 | attgaaaact aaacgacgaa tagcatcaaa tgcttctcac agaggtacac taaaatggaa |
| 10261 | cataatataa aaaattatat agctacagta actaagagaa agcccctaga caatttatac |
| 10321 | aaatgaaacc aagaaagtac gtaagataaa gggaagaagg taacgatggg aataaacacg |
| 10381 | cgcaaagtga gagaaatgaa aggaaaacga attggttgta taacttataa ttaagttact |
| 10441 | tgaagcaatt ctgagaaaca attaaaatca cgttgccaat tttgttgtcg tttgaatatc |
| 10501 | aggcagagca atttatccgc atccaatcga tttttaaaag cccagataaa atatatttat |
| 10561 | ttacgcacta cgttgcgaag gagttttgta aatagttaga acatgcagca gtttcagtat |
| 10621 | atataaatat atagctaagc atttttaaaa ggatatcatt taaagcgctt caagtacgac |
| 10681 | tgcttctgat attacagaca aaccaacaaa ccaaccaacc aaccaatcaa cctattgacg |
| 10741 | tgctcaatat gagagtaaag aatatttcgg catagctacg ggtgtcattg gaaagcaaat |
| 10801 | gcaaagatct tagggattaa cgagtaccaa ctatggcaaa ctacgaagca aaccaaaagc |
| 10861 | agttcaaaaa tcacaatttt agccaattta gtttaaacat ttataaatta taaattattg |
| 10921 | aatctaatgt aatatcttat acacttaaac cgatcgattg aaacgagaag ctagcagttc |
| 10981 | tgagatgagg aaccccagga aaacaaataa caacaaatta acgaatcgta tcaattgtat |
| 11041 | ctcgatcttg cgacaaatct gacttgtgat ttgtgccacg ttttaaatat agatgtctga |
| 11101 | ttagcttgta aacatacata ataaatacga ttaaaaagtc tatacaaaag cccacactca |
| 11161 | gtaagccact acgaagttta tccaaaatga atcgcgactc gcggtagcaa ttgtaaatgt |
| 11221 | ttgacgtggg gagggcgggc gccacgcatc aattacatac caattacaat actaactact |
| 11281 | actttaatac caccactaac acacctacta cgatacgtac taccgtggac ggattgcttg |
| 11341 | tattacgtgt agtctgtaag ggaaattcga gaggtaccat attttaagat gtgcttcgtt |
| 11401 | tgttcaagtc cacccatcca tgacccatga cccatgaacc ctgaccctga actcctgtac |
| 11461 | caaaacccga atcataacca taaccatcca ctcccacaaa agcaaagtct cccacctgcc |
| 11521 | tagcgtcccg tgtgagttgc atgatcttgt atttctattt ggaaactttc tagttattat |
| 11581 | tcaaagacaa ccactttcgt gtaaaaatgt atatgtaata gcataattta agttggcaac |
| 11641 | ccaaaactga ataaacctaa ttctactgat agataactaa tgcgaattgt tttaagttgt |
| 11701 | atttagaacg aaatcgaaat cctcagaact caaatcggtc gcccctcccg caaccaactt |
| 11761 | tccacaacaa caaagcgcag tgtcgtcact tgaattgttc agctaaatgt gtctaaatgt |
| 11821 | aattaaacat aaagagaaaa ctaaagattt ttaaacaaac attgtgagtt ttattaaaca |
| 11881 | tcatcagcat agggagagga gaggacagca gcaaagcaaa ggaaaccaac atctttaata |
| 11941 | tttgatagtt tattaagccc atccattcat agtcgtcata aacattaagt acataaaaga |
| 12001 | aaaacccaac aaaagattca atataaatct cactgaaacg caacaaacaa ccgaaaaccc |
| 12061 | aact |

(SEQ ID NO: 5) (Drosophila melanogaster uncharacterized protein, transcript
variant B (CG11873), mRNA, NCBI Reference Sequence: NM_001276122.1),
which encodes a polypeptide having the amino acid sequence:
MSRRQSLDRPGILSPPGPFLTPSPSPSISASPRLQPSPSLSPFPQDVVLVAGGSQVNANSNPQEHERKAAT
PGRRQSIHQFTNGSPNAGTVVFQPSPSQSPAAATTVIGVTSGGLIATAAGGTGAGLSTGVGAGSSSGVGIG
VALHGNAIGNELNATLVGSNNIQLNKKGTKRATQQQQQQQQQLGHQIFSSTVAPTSISSATAVAGGGGGYG
QFNATAISTPTSFATAGSVVSSSAKGPTKGQKGQQQQQQHQQFQHYQSSSPLIADSPIPSPSGAIAAAAIK
PPTPQPQQMQMLPQQFTISQQPQQQQQAQQVFQFIQGPQGQLIATTPQQQQPIQQQQQQQPQPVQQQQQHR
FVSNAGVTGMSTGKTGKAPQQILPKPQQELQQQKKGTNGGAQISQSAQQTGQANNPTQQQQQQQQQQILLP
ATTNQPQQLLLNQMPVLVQQNPQGVQLILRPPTPQLTTTPSLVIQQNARGQPQLQTQPAQQQLLRIVGING
ATMQLAAAPTFIVSSQANLIHQTAAGQFQSAIKSGTQLTGLHAALAQAQAQAQAPRSQPFATTATLNTQLL
GQSVAAQLQNLQLAAAQIQMPNGLTAISQLPAHLQQSLGGATINLNQLNGAHIQQIANAFQAPQAPGTNSG
GANSTTELFTQSSPAHVPLAVTPEPLRQPTPVPMMQQQQQAAMATIQLQQQQQQQQQAQIQQLQHQLQQTQ
SQVQQAQQSVQATTIPEPKKKPRPRKKKQPPAPTPPAASPTVAPAEVRPPTPQKIAIAATPAPQPTVRAKS
PSPPPTTIQRSSNGKLDLGDVMKFCGITGDDDDDEDYGMGLETGLASESEPAQAHAPATGSAAATTGSSGD
IMISIPNQNGSDGLPFTLTIPAPQPQSSTGSQAAGNESATEIPNILIKIDPSAEAAVPPFGLSTPRLPTAE
EIQKQAQQHLAQQAAAAAAAAKATTATPTINISLPSMTLQPQVAPAPVVTPTPAPSSNQVSTTVVVKPRRK
PAVRRNAKKPDVTSSASNMISSSSGTTTTISSSTSTILNNSQPTTVNTITLTTPTPVTSSSNSTLMLTHGT
PTAVPSQIGNIQISQVHNHHPSALPVSSAVENKIQIMPILPPGATVMGGTPGTGGHAPTTQSTQLVFQPAA
PSVAPTITSDSTGFKLSSDGRQMQLVAIPQATPPSPAASSAQPPVPTPTPALTAGGATILPPQLTGMPANV
SVSVGSPASAAALVPQLTGSLTLTVSEQCERLILRHDPNNPQDHQSQLILQALLKGALPNVTIINEPTRVD
PNKQTTPMLQPQQQVIQIPQPLTQPQQILQQQPKVSTAPKIEVKVANNRKLSGQVTHPASSMLGMTSTTTT
MSTMKPPANLPAKQNEVVSFPQLPLNSQVLIQQQPMISAQLQPQLQPVTVPVSLPTQPVPIPSPAPAPQLA
NNGQQRYIALPPIDPTTQQLFCLNSVTNQITAMSAGQTAASIGPTERLLIAPAGINAQQLAQCLQLGQLHE
NDVNPLPAQQQQQQVATTSSSMTLSMPLRPQPPQQQIVHPIQPITNGLAITTTASSTLTTTTSTTSLTTVT
KQVANVAPIIDQSKAKLEPAKAATSGAGGGAVVKKKPVRKPKATPTNASELANLKNASVVKPMPKLDPLSQ
KPNNNPVQIVQPNVASGKQMIVVGSSSCTTTTTTTMSASIQPFQTTSGTKLVGVTVPMQQQQPTGTAAILQ
QQQQQRTSFSLTATTVATPAPMPSPTVASGVSQLQMQQLPSQQAQQQLQPQQQQQLHAPKQQQPQPNTVSR
VQTIQLTPQMQQIFKQVQMQIQFLTIKLQNKSTFLPVSPEIDSATIAAYNKPMTDAEINLALQRLFAEQHR
ILASGKEVPTPEGMLPTANGTGGFSLMPQPVQQQQQQQQQQLQSTVPEPLKTIVPANVVQPNNHSSTPAGA
ATSGATTAANIQQNITQRIHIYPMQHQQQQQQQQQQQQQQPQHQQQQPQHQLQQTGAKPKQAQPKPPPQQE
QQSQLQIKPKEPANRSKVTSQQLQQNPQPPNGTINMLPQKVSMMPVVQQQQQQQPQQHQQQPMLNKSGPPP
LIFASSTNMLSNSNSNNLHNSNSAMQVNNMLPLPPLQPQQQPQPQLQQPPTPILPPVAPTMAMGVAPGPMG
NLVPSLPTIPSLPLYMPSKMDEMPTQKPKIARLSLFVRQLEVDQESCLKPDYVKPFRTKDEAVKRLIRYHC
MHENDVELPSDEDEEFESTALEFQDKFRQLNGKFQEILMQESTLPHRTSELLQMQQLMIDDLKGEINEIRT
AEKELEQQLKEEQTSEKSTAESDVLSEAKVKEEIKQEPIDKSAQPDGVVDKFDLLKNSADVNKAFSKSQPQ
QTTTVKQEESNESGEPSVNGSVKSEGHDKESSKYIKKDYDNFDIESELTTSFIMKKVENKAALAKSAENRY
QERNMMDQQQQQQHIKANGGDPVDQDDGWYCLQKELNLMNNDHMQQSQQQLNGNQQHLPINRQPAPQQSHF
LDNSNSNNMMGNSNHMSDLFPNGCIDKSNQSTTSSSNSSCVSESLPGSNPVKTVSSCSGQREQPVVMDADQ
QNEHPAISEFFSSDSDVQKSVETRLEAMFGESPVHLDVKSSNDANDIESNLDEIFGDSKSPAANHKSKMSM
WVPDASFMQQAPQQQTSQQQQQYAGPQQPSQQQHHIELNCNNNPRWMQSMEAHYSDFLSTGTSNGELVNGE
SRKRSWDSQLMGSGSDMEDDNSSKRLCTASSSSSSSPMSSQQQHVQVAQHGLLDSDMFPQMLMDQQPHQQQ
QQVQQQHNFAYANIMDQQQQQQHQQQHFQHNLQQQIQQQQQMHVSHMGGAAVVAGGEYDDDISRHVASAID
SILNLQNSGDSLQFSLGSILGDSMLDDQRQAGANLPSCQQEQAIQRRRHLGEEMNDCLISGGGSAVSGVAD
NSSNILLEHHHQQHQLMQSHQQQPHLQHHHHQNMSQAQAQHMGQLNDFSCVAGGLDDPVKSIMTSXLGAPS
STSAAENASNSLILEFNATTL
(SEQ ID NO: 12)

| | |
|---|---|
| 1 | gctcattaac gtttcgcttg gctaagtgcg cacctcgtca ctgaaaccag ttagcaaaag |
| 61 | aaatttttgt tcaagcttaa accgatacct acgtcactat tcgatcattt tgaatgcaaa |
| 121 | ttattcccag attaataaac taggtcgcca tccgtggcgc tgccgccggg ctgcggaccc |
| 181 | gtactttcga cggaaaatat cgggaccatt ttcataaacg atctatgata cagttttaaa |
| 241 | acactttaac gctttccatc gtgcacactt aaaacaaaac tcataaagtg aaagttcata |
| 301 | tcctggcaag taaaaagtga taaatgttaa aacgtctgaa gtgatagtct ggagtttgat |
| 361 | taccaggtga agaagataat atatatggga tgtacaaagt tggcaacgaa gccccccttc |
| 421 | taacaacaat gaaaaaggga gtaatcctac tttctcactt tcaataataa acatacgcac |
| 481 | gtcagccacc gtcgaagtct ccaacgccgc attaaaaata attaattatg atgctaacaa |
| 541 | ctgaacacat aatgcatggg catccccact cgtcagtcgg gcagagtact ctatttgggt |
| 601 | gctccacggc gggccatagc ggaataaatc agctgggcgg cgtatatgtt aatggccggc |
| 661 | cactgcccga ttcaacgcgt caaaaaattg tcgaattggc tcattccggc gcacgtcctt |
| 721 | gtgatatttc aagaatacta caagtgtcca acggttgcgt aagcaaaatt ttgggcagat |
| 781 | attatgaaac tggatcgata aaacctcgag ctataggtgg ttcaaagcca cgagtagcta |
| 841 | caaccccggt tgtgcaaaaa attgcagatt acaaacggga atgtcccagc atatttgcgt |
| 901 | gggaaatacg agatcgactg ctatcggaac aagtttgcaa tagtgataac attccaagtg |
| 961 | tttcatctat taatcgagtc ttacgtaacc tggcctcaca aaaggagcag caagctcagc |
| 1021 | aacaaaacga atccgtttat gaaaagcttc gcatgtttaa tggccaaacg ggcggatggg |
| 1081 | catggtatcc aagcaataca acgacggcac atttgacgct accaccagca gcttccgttg |
| 1141 | tgacatctcc tgcaaattta tcaggacagg ccgatcggga tgatgttcaa aaaagagaat |
| 1201 | tacaattttc agtagaagtt tcgcatacaa actctcacga tagtacatcg gatggaaact |
| 1261 | ctgaacataa ttcatccggg gacgaagact ctcaaatgcg gttgcgccta aaaaggaagt |
| 1321 | tacagcgcaa tcggacatca ttttctaatg agcaaattga cagtcttgaa aaagaatttg |
| 1381 | aaagaacaca ttatcccgat gtttttgcgc gagaaaggct tgctgataaa attggtttgc |
| 1441 | cagaggcacg tattcaggtt tggttttcaa accgacgagc taaatggcgc cgagaagaaa |
| 1501 | aaatgcgaac tcagagacga tcggccgata ccgtggacgg cagtggtcga accagcacgg |
| 1561 | caaataatcc ttcaggaaca actgcatctt cctccgtcgc aacgtcaaac aactcgactc |
| 1621 | cagggattgt gaactcagca atcaacgttg cggaacgaac atcatctgca ttagttagta |
| 1681 | atagccttcc cgaggcttca aatggaccaa ctgttttggg tggtgaagct aatactacac |
| 1741 | acaccagctc tgaaagccca ccccttcagc cagcggcacc gcggctaccc ttaaattctg |
| 1801 | gattcaacac catgtactca tctattccac aaccgattgc aacgatggct gaaaattaca |
| 1861 | actcctcatt aggatcaatg accccgtcat gcttacaaca acgcgatgcc tatccttaca |
| 1921 | tgtttcacga tccgttatca ctaggatctc cctatgtgtc agcccaccat cgaaacacag |
| 1981 | cttgcaaccc ctcagctgcg caccaacagc cccctcagca tggcgtttat accaatagtt |
| 2041 | ctccaatgcc atcatcaaac acaggtgtca tttctgcggg cgtttcggtg cctgtccaga |
| 2101 | tttcaacgca aaatgtatct gacctaacgg gaagcaatta ctggccacgt cttcagtgat |
| 2161 | cgtcaatctt tggctcacca ttagatcatt tgtcaaaggc gactgccgct gcaatcattg |
| 2221 | ccgcacaagc agctgagaaa agccataaac accgaaaaga gcattcaatt gttagtatac |
| 2281 | acaccacaaa aagaaagaaa aacccaccta gtttgaagag acagacaacg tgctgttcat |
| 2341 | ccagatattt aaacatgacc ttcggggcgg ggaaggatgt agatgttgtc ataggccaag |
| 2401 | gtcttatagt gtccttaaca ccacatattt caggaaagcc ggagcttttt ggtagtccta |
| 2461 | acgttaacta ccatcaggac tttcaagtaa tttaaatagg ctgaaccttt tcggtctgaa |
| 2521 | acttgtgaat tgcacactat atgtatttta aaaggcaaat gaaatgttaa atctacatta |
| 2581 | aaattgatgt tatttttgaa taaaaattca agatttgtaa acaatatttc cgcatctttc |
| 2641 | gcatcctttt ttaagttcca aacattcctg atctgctaag ataggttaaa gtatttcggg |
| 2701 | gaatcttcaa ctttactgtt gataagcacg caatcgggat attagacttc tgcagttgct |
| 2761 | tgcaattttt taagccttca caattatatt tatattttga accatttttt gatattataa |
| 2821 | attaaactta ttcatgtatt aatactattt attaatcgtc tttaagtaaa gaatgagcaa |
| 2881 | ttatgctcat acatacttaa aaaaaaaacc atgtgttacc aaacttaggg aagtgcgtgg |
| 2941 | aagacggata taaagcaaag tgacgttata catgatttta catttacatt aaattcttat |
| 3001 | atgacaattt atgtttttag agtcgtaggc gttatatagt ttcttctact agtaacgtac |
| 3061 | ttctaaacaa atctctcata cccttttcct ctacgattat atcaccaaat tgttttgttt |
| 3121 | cccaaatata accattgcat tttgaattaa aaattacatt tttttaagtt caataaaaac |
| 3181 | taccagataa tgctaattaa aggtcttata aacattttga cgaaattatg actttgaatt |
| 3241 | atatatatac atttaagtac acgaacaatt tctactgtac gtaactatta aaagatatag |
| 3301 | tgtttctaat ttgatcggca tgaaataaac taaaaccact gattcatttg ctagaaaacc |
| 3361 | cataaacctc ataaaaataa ataaaaaaca tttcgcgcat actggtcgat atgttttaaa |
| 3421 | aattgtatat tttattataa tgtattcaaa ttataaatta tctgtacatg attcttttaa |
| 3481 | atgtaagcta ttagttatta atccatgtgt acaccgtttg taaaacagaa acttataaca |
| 3541 | acgtaagatt gaatgaattg caacgaactt gactccaatg aaggaaatga gctaacatct |
| 3601 | catccagata aacaccaacc ccaacaagcc ttcaggttta tatcataata ataaaatgtc |
| 3661 | gtatatgtat aatgtatggt tcaaatttat atttagaata aattaatgta acatttcaga |
| 3721 | atatacaggt gtatctgttg ttcttaaaag gggcttaatg aaatccaact ttaataaaaa |
| 3781 | acaagagaga atgctatagt cgagttcgtc gactattaga tacccgttac tcagctagtg |
| 3841 | tgtgaccgtt ttggctgcaa aaaacgatag aaatttacac aactaataaa atttaaaaaa |
| 3901 | aaaatattca aaaaaaattt c |

(SEQ ID NO: 6) (Drosophila melanogaster twin of eyeless (toy), transcript
variant D, mRNA, NCBI Reference Sequence: NM_001382011.1), which
encodes a polypeptide having the amino acid sequence
MMLTTEHIMHGHPHSSVGQSTLFGCSTAGHSGINQLGGVYVNGRPLPDSTRQKIVELAHSGARP
CDISRILQVSNGCVSKILGRYYETGSIKPRAIGGSKPRVATTPVVQKIADYKRECPSIFAWEIR
DRLLSEQVCNSDNIPSVSSINRVLRNLASQKEQQAQQQNESVYEKLRMENGQTGGWAWYPSNTT
TAHLTLPPAASVVTSPANLSGQADRDDVQKRELQFSVEVSHTNSHDSTSDGNSEHNSSGDEDSQ
MRLRLKRKLQRNRTSFSNEQIDSLEKEFERTHYPDVFARERLADKIGLPEARIQVWESNRRAKW
RREEKMRTQRRSADTVDGSGRTSTANNPSGTTASSSVATSNNSTPGIVNSAINVAERTSSALVS
NSLPEASNGPTVLGGEANTTHTSSESPPLQPAAPRLPLNSGENTMYSSIPQPIATMAENYNSSL
GSMTPSCLQQRDAYPYMFHDPLSLGSPYVSAHHRNTACNPSAAHQQPPQHGVYTNSSPMPSSNT
GVISAGVSVPVQISTQNVSDLTGSNYWPRLQXSSIFGSPLDHLSKATAAAIIAAQAAEKSHKHR
KEHSIVSIHTTKRKKNPPSLKRQTTCCSSRYLNMTFGAGKDVDVVIGQGLIVSLTPHISGKPEL
FGSPNVNYHQDFQVI
(SEQ ID NO: 13)

The Drosophila Pax6 paralogs have different functions in head development but can partially substitute for each other. See, e.g., Jacobsson, et al., “The Drosophila Pax6 paralogs have different functions in head development but can partially substitute for each other,” Mol Genet Genomics. 2009 September; 282(3): 217-231; Published online 2009 May 30. doi: 10.1007/s00438-009-0458-2; ey eyeless [Drosophila melanogaster (fruit fly), Gene ID: 43812; and toy twin of eyeless [Drosophila melanogaster (fruit fly) Gene ID: 43833

### 2. MYB Sequences

Nucleic acid and amino acid sequences for Myb are known in the art. See, non-limiting example, NCBI Accession No. NM_001130173.2 and Gene ID: 4602 which re specifically incorporated by references herein, and including all of the sequence provided, cited, and referenced therein.

| | |
|---|---|
| 1 | acactttaat atcaacctgt ttcctcctcc tccttctcct cctcctccgt gacctcctcc |
| 61 | tcctctttct cctgagaaac ttcgccccag cggtgcggag cgccgctgcg cagccgggga |
| 121 | gggacgcagg caggcggcgg gcagcgggag gcggcagccc ggtgcggtcc ccgcggctct |
| 181 | cggcggagcc ccgcgcccgc cgcgccatgg cccgaagacc ccggcacagc atatatagca |
| 241 | gtgacgagga tgatgaggac tttgagatgt gtgaccatga ctatgatggg ctgcttccca |
| 301 | agtctggaaa gcgtcacttg gggaaaacaa ggtggacccg ggaagaggat gaaaaactga |
| 361 | agaagctggt ggaacagaat ggaacagatg actggaaagt tattgccaat tatctcccga |
| 421 | atcgaacaga tgtgcagtgc cagcaccgat ggcagaaagt actaaaccct gagctcatca |
| 481 | agggtccttg gaccaaagaa gaagatcaga gagtgataga gcttgtacag aaatacggtc |
| 541 | cgaaacgttg gtctgttatt gccaagcact taaaggggag aattggaaaa caatgtaggg |
| 601 | agaggtggca taaccacttg aatccagaag ttaagaaaac ctcctggaca gaagaggaag |
| 661 | acagaattat ttaccaggca cacaagagac tggggaacag atgggcagaa atcgcaaagc |
| 721 | tactgcctgg acgaactgat aatgctatca agaaccactg gaattctaca atgcgtcgga |
| 781 | aggtcgaaca ggaaggttat ctgcaggagt cttcaaaagc cagccagcca gcagtggcca |
| 841 | caagcttcca gaagaacagt catttgatgg gttttgctca ggctccgcct acagctcaac |
| 901 | tccctgccac tggccagccc actgttaaca acgactattc ctattaccac atttctgaag |
| 961 | cacaaaatgt ctccagtcat gttccatacc ctgtagcgtt acatgtaaat atagtcaatg |
| 1021 | tccctcagcc agctgccgca gccattcaga gacactataa tgatgaagac cctgagaagg |
| 1081 | aaaagcgaat aaaggaatta gaattgctcc taatgtcaac cgagaatgag ctaaaaggac |
| 1141 | agcaggtgct accaacacag aaccacacat gcagctaccc cgggtggcac agcaccacca |
| 1201 | ttgccgacca caccagacct catggagaca gtgcacctgt ttcctgtttg ggagaacacc |
| 1261 | actccactcc atctctgcca gcggatcctg gctccctacc tgaagaaagc gcctcgccag |
| 1321 | caaggtgcat gatcgtccac cagggcacca ttctggataa tgttaagaac ctcttagaat |
| 1381 | ttgcagaaac actccaattt atagattctg attcttcatc atggtgtgat ctcagcagtt |
| 1441 | ttgaattctt tgaagaagca gatttttcac ctagccaaca tcacacaggc aaagccctac |
| 1501 | agcttcagca aagagagggc aatgggacta aacctgcagg agaacctagc ccaagggtga |
| 1561 | acaaacgtat gttgagtgag agttcacttg acccacccaa ggtcttacct cctgcaaggc |
| 1621 | acagcacaat tccactggtc atccttcgaa aaaaacgggg ccaggccagc cccttagcca |
| 1681 | ctggagactg tagctccttc atatttgctg acgtcagcag ttcaactccc aagcgttccc |
| 1741 | ctgtcaaaag cctacccttc tctccctcgc agttcttaaa cacttccagt aaccatgaaa |
| 1801 | actcagactt ggaaatgcct tctttaactt ccacccccct cattggtcac aaattgactg |
| 1861 | ttacaacacc atttcataga gaccagactg tgaaaactca aaaggaaaat actgttttta |
| 1921 | gaaccccagc tatcaaaagg tcaatcttag aaagctctcc aagaactcct acaccattca |
| 1981 | aacatgcact tgcagctcaa gaaattaaat acggtcccct gaagatgcta cctcagacac |
| 2041 | cctctcatct agtagaagat ctgcaggatg tgatcaaaca ggaatctgat gaatctggaa |
| 2101 | ttgttgctga gtttcaagaa aatggaccac ccttactgaa gaaaatcaaa caagaggtgg |
| 2161 | aatctccaac tgataaatca ggaaacttct tctgctcaca ccactgggaa ggggacagtc |
| 2221 | tgaataccca actgttcacg cagacctcgc ctgtggcaga tgcaccgaat attcttacaa |
| 2281 | gctccgtttt aatggcacca gcatcagaag atgaagacaa tgttctcaaa gcatttacag |
| 2341 | tacctaaaaa caggtccctg gcgagcccct tgcagccttg tagcagtacc tgggaacctg |
| 2401 | catcctgtgg aaagatggag gagcagatga catcttccag tcaagctcgt aaatacgtga |
| 2461 | atgcattctc agcccggacg ctggtcatgt gagacatttc cagaaaagca ttatggtttt |
| 2521 | cagaacactt caagttgact tgggatatat cattcctcaa catgaaactt ttcatgaatg |
| 2581 | ggagaagaac ctatttttgt tgtggtacaa cagttgagag cagcaccaag tgcatttagt |
| 2641 | tgaatgaagt cttcttggat ttcacccaac taaaaggatt tttaaaaata aataacagtc |
| 2701 | ttacctaaat tattaggtaa tgaattgtag ccagttgtta atatcttaat gcagattttt |
| 2761 | ttaaaaaaaa cataaaatga tttatctgta ttttaaagga tccaacagat cagtattttt |
| 2821 | tcctgtgatg ggttttttga aatttgacac attaaaaggt actccagtat ttcacttttc |
| 2881 | tcgatcacta aacatatgca tatattttta aaaatcagta aaagcattac tctaagtgta |
| 2941 | gacttaatac catgtgacat ttaatccaga ttgtaaatgc tcatttatgg ttaatgacat |
| 3001 | tgaaggtaca tttattgtac caaaccattt tatgagtttt ctgttagctt gctttaaaaa |
| 3061 | ttattactgt aagaaatagt tttataaaaa attatatttt tattcagtaa tttaattttg |
| 3121 | taaatgccaa atgaaaaacg ttttttgctg ctatggtctt agcctgtaga catgctgcta |
| 3181 | gtatcagagg ggcagtagag cttggacaga aagaaaagaa acttggtgtt aggtaattga |
| 3241 | ctatgcacta gtatttcaga ctttttaatt ttatatatat atacattttt tttccttctg |
| 3301 | caatacattt gaaaacttgt ttgggagact ctgcattttt tattgtggtt tttttgttat |
| 3361 | tgttggttta tacaagcatg cgttgcactt cttttttggg agatgtgtgt tgttgatgtt |
| 3421 | ctatgtttt ttttgagtgt agcctgactg ttttataatt tgggagttct gcatttgatc |
| 3481 | cgcatcccct gtggtttcta agtgtatggt ctcagaactg ttgcatggat cctgtgtttg |
| 3541 | caactgggga gacagaaact gtggttgata gccagtcact gccttaagaa catttgatgc |
| 3601 | aagatggcca gcactgaact tttgagatat gacggtgtac ttactgcctt gtagcaaaat |
| 3661 | aaagatgtgc ccttatttta ccta |

(SEQ ID NO: 7), (Homo sapiens MYB proto-oncogene, transcription factor
(MYB), transcript variant 1, mRNA
NCBI Reference Sequence: NM_001130173.2), which encodes a polypeptide
having the amino acid sequence
MARRPRHSIYSSDEDDEDFEMCDHDYDGLLPKSGKRHLGKTRWTREEDEKLKKLVEQNGTDDWK
VIANYLPNRTDVQCQHRWQKVLNPELIKGPWTKEEDQRVIELVQKYGPKRWSVIAKHLKGRIGK
QCRERWHNHLNPEVKKTSWTEEEDRIIYQAHKRLGNRWAEIAKLLPGRTDNAIKNHWNSTMRRK
VEQEGYLQESSKASQPAVATSFQKNSHLMGFAQAPPTAQLPATGQPTVNNDYSYYHISEAQNVS
SHVPYPVALHVNIVNVPQPAAAAIQRHYNDEDPEKEKRIKELELLLMSTENELKGQQVLPTQNH
TCSYPGWHSTTIADHTRPHGDSAPVSCLGEHHSTPSLPADPGSLPEESASPARCMIVHQGTILD
NVKNLLEFAETLQFIDSDSSSWCDLSSFEFFEEADESPSQHHTGKALQLQQREGNGTKPAGEPS
PRVNKRMLSESSLDPPKVLPPARHSTIPLVILRKKRGQASPLATGDCSSFIFADVSSSTPKRSP
VKSLPFSPSQFLNTSSNHENSDLEMPSLTSTPLIGHKLTVTTPFHRDQTVKTQKENTVERTPAI
KRSILESSPRTPTPFKHALAAQEIKYGPLKMLPQTPSHLVEDLQDVIKQESDESGIVAEFQENG
PPLLKKIKQEVESPTDKSGNFFCSHHWEGDSLNTQLFTQTSPVADAPNILTSSVLMAPASEDED
NVLKAFTVPKNRSLASPLQPCSSTWEPASCGKMEEQMTSSSQARKYVNAFSARTLVM
(SEQ ID NO: 14).

### B. Pax6 Inhibitors

Compounds for decreasing the expression, activity, and/or bioavailability of Pax6, and formulations formed therewith are provided. In some embodiments, the compound is an inhibitory polypeptide; a small molecule or peptidomimetic, or an inhibitory nucleic acid that targets genomic or expressed Pax6 nucleic acids (e.g., Pax6 mRNA), or a vector that encodes an inhibitory nucleic acid. The compound can reduce the expression or bioavailability of Pax6. Pax6 inhibition can be competitive, non-competitive, uncompetitive, or product inhibition. Thus, an Pax6 inhibitor can directly inhibit Pax6, a Pax6 inhibitor can inhibit another factor in a pathway that leads to induction, persistence, or amplification of Pax6 expression, or a combination thereof, for example other pathway members discussed herein, such as c-Myb (e.g., cMyb mRNA).

In other embodiments, the methods are carried out by targeting a target of Pax6. The Examples below show that Pax6 induces Tau phosphorylation by increasing expression of one or more of Cdk5, p35, GSK-3β, mitogen-activated protein kinases (MAPK), serine/threonine protein kinase (MARK), calcium/calmodulin-dependent protein kinase type II a (CAMK2a), etc. Thus, in some embodiments the compound for use in the disclosed methods is an inhibitor of one or more of these targets.

Exemplary inhibitors are described below.

### 1. Pharmacological Pax6 Inhibitors

In some embodiments, the inhibitor is a small molecule. Exemplary small molecule compounds include, but are not limited to, palbociclib, abemaciclib, ribociclib, flavopiridol, apigenin, and ICCB280, as well as prodrugs, analogues and other structural variants, tautomers, isomers, epimers, diastereoisomer, as well as any form of the compounds, such as the base (zwitter ion), pharmaceutically acceptable salts, e.g., pharmaceutically acceptable acid addition salts, hydrates or solvates of the base or salt, as well as anhydrates, and also amorphous, or crystalline forms thereof.

Palbociclib (IBRANCE), abemaciclib, and ribociclib are selective inhibitors of the cyclin-dependent kinases CDK4 and CDK6. Palbociclib was the first CDK4/6 inhibitor to be approved as a cancer therapy. See, e.g., Liu, et al., “Mechanisms of the CDK4/6 inhibitor palbociclib (PD 0332991) and its future application in cancer treatment,” Oncol Rep, 2018 March; 39(3):901-911. doi: 10.3892/or.2018.6221. Epub 2018 Jan. 19. and U.S. Pat. Nos. 6,936,612, 7,208,489, 7,456,168, 10,723,730, RE47,739, 7,855,211, 8,324,225, 8,415,355, 8,685,980, 8,962,630, 9,193,732, 9,416,136, 9,868,739, 10,799,506 each of which is specifically incorporated by reference herein in its entirety.

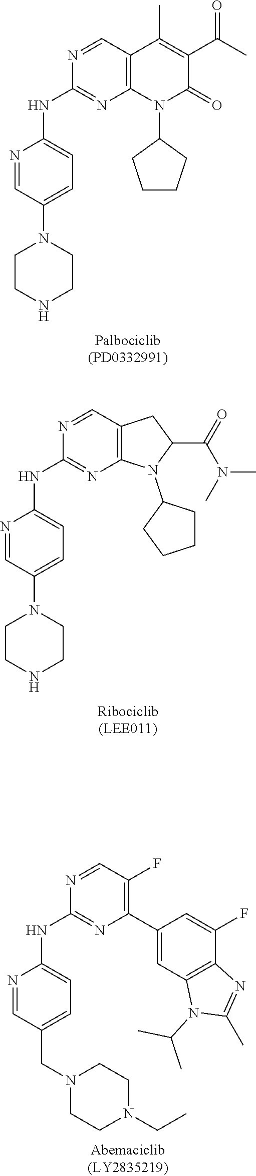 [SMILES: CC(=O)c1c(C)c2cnc(Nc3ccc(N4CCNCC4)cn3)nc2n(C2CCCC2)c1=O ; CN(C)C(=O)C1Cc2cnc(Nc3ccc(N4CCNCC4)cn3)nc2N1C1CCCC1 ; CCN1CCN(Cc2ccc(Nc3ncc(F)c(-c4cc(F)c5nc(C)n(C(C)C)c5c4)n3)nc2)CC1]

Apigenin (4′,5,7-trihydroxyflavone) is found in many plants, is a natural product belonging to the flavone class that is the aglycone of several naturally occurring glycosides. Apigenin is found in many fruits and vegetables, but parsley, celery, celeriac, and chamomile tea are the most common sources. See, e.g., Salehi, et al., “The Therapeutic Potential of Apigenin,” Int J Mol Sci. 2019 March; 20(6): 1305, which is specifically incorporated by reference herein in its entirety.

Flavopiridol (HMR 1275, L86-8275), a flavonoid derived from an indigenous plant from India, demonstrated potent and specific in vitro inhibition of all cdks tested (cdks 1, 2, 4 and 7) with clear block in cell cycle progression at the G1/S and G2/M boundaries, and became the first cyclin-dependent kinase inhibitor in human clinical trials. See, Senderowicz, et al., “Flavopiridol: the first cyclin-dependent kinase inhibitor in human clinical trials,” Invest New Drugs. 1999; 17(3):313-20. doi: 10.1023/a:1006353008903.

ICCB280, having the structure

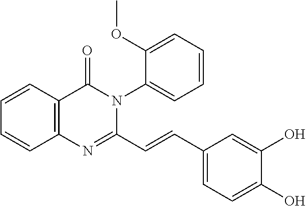 [SMILES: COc1ccccc1-n1c(/C=C/c2ccc(O)c(O)c2)nc2ccccc2c1=O]

is a potent inducer of C/EBPa. ICCB280 exhibits anti-leukemic properties including terminal differentiation, proliferation arrest, and apoptosis through activation of C/EBPu and affecting its downstream targets (such as C/EBPE, G-CSFR and c-Myc). It believed that ICCB280 will also up-regulates CEBP-alpha expression then CEBP-alpha protein will interact with CDK4 to inhibit down stream genes of E2F1, c-Myb, Pax6, Gsk3-beta, P-Tau and total Tau in Tau-related disorders (e.g., Alzheimer's disease, FTLD, autism, epilepsy, stroke, Dravet syndrome and seizures, etc.).

See, Radomska, et al., “A Cell-Based High-Throughput Screening for Inducers of Myeloid Differentiation,” J Biomol Screen. 2015 October; 20(9): 1150-1159; Kobayashi and Takei, “[Transcription factor-based therapies for acute myeloid leukemia],” Rinsho Ketsueki, 2018; 59(7):922-931. doi: 10.11406/rinketsu.59.922.

In some embodiments, the Pax6 inhibitor is an E2F1 inhibitor. E2F1 inhibitors include, but are not limited to, diclofenac, indomethacin, non-steroidal anti-inflammatory (NSAIDs) drugs, (−)-kusunokinin, bortezomib (BZB), valproic acid (VPA), bigelovin, eugenol, emodin, icilin, NSC69603, gambogic acid, tolfenamic acid, HDAC inhibitors such as oxamflatin, 4-Allyl-2-methoxyphenol (eugenol), piperlongumine, Delta 9-tetrahydrocannabinol, bortezomib, sorafenib, dracorhodin perchlorate, triptolide fangchinoline, PD-0332991, or methyl gallate.

See, e.g., Valle, et al., “Non-Steroidal Anti-inflammatory Drugs Decrease E2F1 Expression and Inhibit Cell Growth in Ovarian Cancer Cells”, PLoS One. 2013; 8(4): e61836; Tedasen, et al., “(−)-Kusunokinin inhibits breast cancer in N-nitrosomethylurea-induced mammary tumor rats”, Eur J Pharmacol., 2020 Sep. 5; 882:173311. doi: 10.1016/j.ejphar.2020.173311. Epub 2020 Jun. 30; Farra, et al., “Impairment of the Pin1/E2F1 axis in the anti-proliferative effect of bortezomib in hepatocellular carcinoma cells,” Biochimie, 2015 May; 112:85-95. doi: 10.1016/j.biochi.2015.02.015. Epub 2015 Mar. 3; Fang, et al., “Valproic acid suppresses Warburg effect and tumor progression in neuroblastoma,” Biochem Biophys Res Commun, 2019 Jan. 1; 508(1):9-16. doi: 10.1016/j.bbrc.2018.11.103. Epub 2018 Nov. 20; Liu, et al., “Small compound bigelovin exerts inhibitory effects and triggers proteolysis of E2F1 in multiple myeloma cells,” Cancer Sci., 2013 December; 104(12):1697-704. doi: 10.1111/cas.12295. Epub 2013 Nov. 8; Al-Sharif, et al., “Eugenol triggers apoptosis in breast cancer cells through E2F1/survivin down-regulation,” BMC Cancer, 2013 Dec. 13; 13:600. doi: 10.1186/1471-2407-13-600; Xu, et al., “Emodin as a selective proliferative inhibitor of vascular smooth muscle cells versus endothelial cells suppress arterial intima formation,” Life Sci, 2018 Aug. 15; 207:9-14. doi: 10.1016/j.lfs.2018.05.042. Epub 2018 May 24; Lee, et al., “Icilin inhibits E2F1-mediated cell cycle regulatory programs in prostate cancer,” Biochem Biophys Res Commun, 2013 Nov. 29; 441(4):1005-10. doi: 10.1016/j.bbrc.2013.11.015. Epub 2013 Nov. 12; Martirosyan, et al., “Differentiation-inducing quinolines as experimental breast cancer agents in the MCF-7 human breast cancer cell model,” Biochem Pharmacol., 2004 Nov. 1; 68(9):1729-38. doi: 10.1016/j.bcp.2004.05.003; Xia, et al., “Gambogic acid sensitizes gemcitabine efficacy in pancreatic cancer by reducing the expression of ribonucleotide reductase subunit-M2 (RRM2),” J Exp Clin Cancer Res., 2017 Aug. 10; 36(1):107. doi: 10.1186/s13046-017-0579-0; Sankpal, et al., “Small molecule tolfenamic acid inhibits PC-3 cell proliferation and invasion in vitro, and tumor growth in orthotopic mouse model for prostate cancer,” Prostate, 2012 November; 72(15):1648-58. doi: 10.1002/pros.22518. Epub 2012 Apr. 2; Wang, et al., “HDAC Inhibitor Oxamflatin Induces Morphological Changes and has Strong Cytostatic Effects in Ovarian Cancer Cell Lines,” Curr Mol Med., 2016; 16(3):232-42. doi: 10.2174/1566524016666160225151408; Ghosh, et al., “Eugenol causes melanoma growth suppression through inhibition of E2F1 transcriptional activity,” J Biol Chem., 2005 Feb. 18; 280(7):5812-9. doi: 10.1074/jbc.M411429200. Epub 2004 Dec. 1; Han, et al., “Piperlongumine inhibits proliferation and survival of Burkitt lymphoma in vitro,” Leuk Res., 2013 February; 37(2):146-54. doi: 10.1016/j.leukres.2012.11.009. Epub 2012 Dec. 10; Galanti, et al., “Delta 9-tetrahydrocannabinol inhibits cell cycle progression by downregulation of E2F1 in human glioblastoma multiforme cells,” Acta Oncol., 2008; 47(6):1062-70. doi: 10.1080/02841860701678787; Baiz, et al., “Bortezomib arrests the proliferation of hepatocellular carcinoma cells HepG2 and JHH6 by differentially affecting E2F1, p21 and p27 levels,” Biochimie, 2009 March; 91(3):373-82. doi: 10.1016/j.biochi.2008.10.015. Epub 2008 Nov. 12; Zhai, et al., “Sorafenib enhances the chemotherapeutic efficacy of S-1 against hepatocellular carcinoma through downregulation of transcription factor E2F-1,” Cancer Chemother Pharmacol., 2013 May; 71(5):1255-64. doi: 10.1007/s00280-013-2120-2. Epub 2013 Feb. 23; Rasul, et al., “Dracorhodin perchlorate inhibits PI3K/Akt and NF-κB activation, up-regulates the expression of p53, and enhances apoptosis,” Apoptosis, 2012 October; 17(10):1104-19. doi: 10.1007/s10495-012-0742-1; Oliveira, et al., “Triptolide abrogates growth of colon cancer and induces cell cycle arrest by inhibiting transcriptional activation of E2F,” Lab Invest., 2015 June; 95(6):648-659. doi: 10.1038/labinvest.2015.46. Epub 2015 Apr. 20; Luo, et al., “Fangchinoline inhibits the proliferation of SPC-A-1 lung cancer cells by blocking cell cycle progression,” Exp Ther Med., 2016 February; 11(2):613-618. doi: 10.3892/etm.2015.2915. Epub 2015 Dec. 4; Logan, et al., “PD-0332991, a potent and selective inhibitor of cyclin-dependent kinase 4/6, demonstrates inhibition of proliferation in renal cell carcinoma at nanomolar concentrations and molecular markers predict for sensitivity,” Anticancer Res., 2013 August; 33(8):2997-3004; Rahman, et al., “Methyl gallate, a potent antioxidant inhibits mouse and human adipocyte differentiation and oxidative stress in adipocytes through impairment of mitotic clonal expansion,” Biofactors, 2016 Nov. 12; 42(6):716-726. doi: 10.1002/biof.1310. Epub 2016 Jul. 13, each of which is specifically incorporated by reference herein in its entirety.

### 2. Functional Nucleic Acids Inhibitors of Pax6

The Pax6 inhibitor can be a functional nucleic acid. Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, shRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Therefore the compositions can include one or more functional nucleic acids designed to reduce expression of the Pax6 gene, or a gene product thereof. For example, the functional nucleic acid or polypeptide can be designed to target and reduce or inhibit expression or translation of Pax6 mRNA; or to reduce or inhibit expression, reduce activity, or increase degradation of Pax6 protein. In some embodiments, the composition includes a vector suitable for in vivo expression of the functional nucleic acid.

In some embodiments, a functional nucleic acid or polypeptide is designed to target a segment of the nucleic acid sequence of any of SEQ ID NOS:1-7, or the complement thereof, or a genomic sequence corresponding therewith, or variants thereof having a nucleic acid sequence at least 65%, 70%, 71%, 72%, 73%, 74%, 75%, 76%, 77%, 78%, 79% 80%, 8%1%, 82%, 83%, 84%, 85%, 86%, 87%, 88%, 89%, 90%, 91%, 92%, 93%, 94%, 95%, 96%, 97%, 98%, 99% identical to any of SEQ ID NOS:1-7.

In a particular embodiments, the Pax6 inhibitor is an peptide inhibitor of E2F, such as PEP:

(SEQ ID NO15)
L-Arg PEP Penetratin-HHHRLSH
(SEQ ID NO: 15)
D-Arg PEP Penetratin-HHH(D)RLSH

See, e.g., Shaik, et al., “Modeling and antitumor studies of a modified L-penetratin peptide targeting E2F in lung cancer and prostate cancer,” Oncotarget. 2018 Sep. 7; 9(70): 33249-33257, Published online 2018 Sep. 7. doi: 10.18632/oncotarget.26064; and Xie, et al., “A novel peptide that inhibits E2F transcription and regresses prostate tumor xenografts,” Oncotarget. 2014; 5:901-doi.org/10.18632/oncotarget.1809.

In some embodiments, a functional nucleic acid or polypeptide is designed to target a segment of a the nucleic acid encoding the amino acid sequence of any of SEQ ID NOS:8-14, or the complement thereof, or variants thereof having a nucleic acid sequence 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a nucleic acid encoding the amino acid sequence of any of SEQ ID NOS:8-14.

In some embodiments, the function nucleic acid hybridizes to the nucleic acid of any of SEQ ID NOS:1-7, or a complement thereof, for example, under stringent conditions. In some embodiments, the functional nucleic acid hybridizes to a nucleic acid sequence that encodes any of SEQ ID NOS:1-7, or a complement thereof, for example, under stringent conditions.

a. Antisense

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

b. Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

c. Ribozymes

The functional nucleic acids can be ribozymes. Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

d. Triplex Forming Oligonucleotides

The functional nucleic acids can be triplex forming molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

e. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

f. RNA Interference

In some embodiments, the functional nucleic acids induce gene silencing through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Texas), ChemGenes (Ashland, Massachusetts), Dharmacon (Lafayette, Colorado), Glen Research (Sterling, Virginia), MWB Biotech (Esbersberg, Germany), Proligo (Boulder, Colorado), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors having shRNA are available, such as, for example, Imgenex's GENESUPPRESSOR™ Construction Kits and Invitrogen's BLOCK-IT™ inducible RNAi plasmid and lentivirus vectors.

In some embodiment, the functional nucleic acid is siRNA, shRNA, miRNA. In some embodiments, the composition includes a vector expressing the functional nucleic acid. Methods of making and using vectors for in vivo expression of functional nucleic acids such as antisense oligonucleotides, siRNA, shRNA, miRNA, EGSs, ribozymes, and aptamers are known in the art.

The experiments below show that total Tau decreased after Pax6 downregulation in the RNA sequence data and western blot data. It was also observed that several potential MicroRNAs (miRNAs) regulating tau expression were elevated (fold increase>2) after siRNA Pax6 knockdown including, miR-670 (9-fold), miR-34a (6-fold) miR-16, and miR-692 (2-fold) in the RNA-seq data. In some embodiments, the compositions are or include any of the miRNA, or an expression construct encoding the same.

A dose dependent delivery of miR-16 into mouse brain has been shown to downregulate total tau expression in cortex, brainstem, and striatum (Parsi, et al., “Preclinical Evaluation of miR-15/107 Family Members as Multifactorial Drug Targets for Alzheimer's Disease,” Mol Ther Nucleic Acids, 2015 Oct. 6; 4(10):e256. doi: 10.1038/mtna.2015.33.). Overexpression of miR-34 reduces tau expression in cultured cells (Dickson, et al., “Alternative polyadenylation and miR-34 family members regulate tau expression”, J Neurochem, 2013 December; 127(6):739-49. doi: 10.1111/jnc.12437. Epub 2013 Sep. 18.).

g. Small Activating RNA

In yet another embodiment, the function nucleic acid is a small activating RNA (saRNA), most particularly CEBPA-saRNA. It is believed that using a CEBP-alpha Small Active RNA (CEBPA-saRNA) (Reebye, et al., “Gene activation of CEBPA using saRNA: preclinical studies of the first in human saRNA drug candidate for liver cancer,” Oncogene volume 37, pages 3216-3228 (2018)) will up regulate CEBP-alpha expression then CEBP-alpha protein will interact with CDK4 (Wang, et al., “C/EBPalpha triggers proteasome-dependent degradation of cdk4 during growth arrest,” EMBO J, 2002 Mar. 1; 21(5):930-41. doi: 10.1093/emboj/21.5.930.) to inhibit down stream genes of E2F1, c-Myb, pax6, Gsk3-beta, p-Tau and total Tau in those disorders (e.g., Alzheimer's disease, FTLD, autism, epilepsy, stroke, Dravet syndrome and seizures, etc.).

See Reebye, et al., “Gene activation of CEBPA using saRNA: preclinical studies of the first in human saRNA drug candidate for liver cancer,” Oncogene volume 37, pages 3216-3228 (2018), Wang, et al., “C/EBPalpha triggers proteasome-dependent degradation of cdk4 during growth arrest,” EMBO J, 2002 Mar. 1; 21(5):930-41. doi: 10.1093/emboj/21.5.930, Sarker, et al., “MTL-CEBPA, a Small Activating RNA Therapeutic Upregulating C/EBP-α, in Patients with Advanced Liver Cancer: A First-in-Human, Multicenter, Open-Label, Phase I Trial”, Clin Cancer Res, 2020 Aug. 1; 26(15):3936-3946. doi: 10.1158/1078-0432.CCR-20-0414; Zhou, et al., “Anti-inflammatory Activity of MTL-CEBPA, a Small Activating RNA Drug, in LPS-Stimulated Monocytes and Humanized Mice,” Mol Ther, 2019 May 8; 27(5):999-1016. doi: 10.1016/j.ymthe.2019.02.018. Epub 2019 Feb. 26, Yoon, et al., “Targeted Delivery of C/EBPα-saRNA by RNA Aptamers Shows Anti-tumor Effects in a Mouse Model of Advanced PDAC,” Mol Ther Nucleic Acids, 2019 Dec. 6; 18:142-154. doi: 10.1016/j.omtn.2019.08.017. Epub 2019 Aug. 22; Kwok, et al., “Developing small activating RNA as a therapeutic: current challenges and promises,” Ther Deliv, 2019 March; 10(3):151-164. doi: 10.4155/tde-2018-0061.

### C. Isolated Nucleic Acid Molecules

Isolated nucleic acid sequences encoding Pax6 proteins, polypeptides, fusions fragments and variants thereof, as well as inhibitor nucleic acid, and vectors and other expression constructs encoding the foregoing are also disclosed herein. As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-Pax6 proteins). The term “isolated” as used herein with respect to nucleic acids also includes the combination with any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The nucleic acid sequences encoding Pax6 polypeptides include genomic sequences. Also disclosed are mRNA sequence wherein the exons have been deleted. Other nucleic acid sequences encoding Pax6 polypeptides, such polypeptides that include the above-identified amino acid sequences and fragments and variants thereof, are also disclosed. Nucleic acids encoding Pax6 polypeptides may be optimized for expression in the expression host of choice. Codons may be substituted with alternative codons encoding the same amino acid to account for differences in codon usage between the organism from which the Pax6 nucleic acid sequence is derived and the expression host. In this manner, the nucleic acids may be synthesized using expression host-preferred codons.

Nucleic acids can be in sense or antisense orientation, or can be complementary to a reference sequence encoding a Pax6 polypeptide. Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Common modifications are discussed in more detail below.

Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

### 1. Vectors and Host Cells

Vectors encoding Pax6 polypeptides, fusion, fragments, and variants and inhibitor nucleic acids thereof are also provided. Nucleic acids, such as those described above, can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the Pax6 polypeptides or fusion polypeptides described herein.

The vectors can be used to express Pax6 or nucleic acids inhibitory thereof in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active DNA into mammalian somatic tissue or organ in vivo.

Nucleic acids may also be administered in vivo by viral means. Nucleic acid molecules encoding polypeptides or fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

### 2. Oligonucleotide Composition

The disclosed nucleic acids nucleic acids can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone.

a. Heterocyclic Bases

The principal naturally-occurring nucleotides include uracil, thymine, cytosine, adenine and guanine as the heterocyclic bases. The oligonucleotides can include chemical modifications to their nucleobase constituents. Chemical modifications of heterocyclic bases or heterocyclic base analogs may be effective to increase the binding affinity or stability in binding a target sequence. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-.beta.-D-ribofuranosyl)pyridine (2-aminopyridine), and various pyrrolo- and pyrazolopyrimidine derivatives.

b. Sugar Modifications

Oligonucleotides can also contain nucleotides with modified sugar moieties or sugar moiety analogs. Sugar moiety modifications include, but are not limited to, 2′-O-aminoetoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

In some embodiments, the nucleic acid is a morpholino oligonucleotide. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tₘ, even with oligomers as short as 10-14 bases; the ability of the oligomer to be actively transported into mammalian cells; and the ability of an oligomer:RNA heteroduplex to resist RNAse degradation.

In some embodiments, oligonucleotides employ morpholino-based subunits bearing base-pairing moieties, joined by uncharged linkages, as described above.

c. Internucleotide Linkages

Oligonucleotides connected by an internucleotide bond that refers to a chemical linkage between two nucleoside moieties. Modifications to the phosphate backbone of DNA or RNA oligonucleotides may increase the binding affinity or stability oligonucleotides, or reduce the susceptibility of oligonucleotides nuclease digestion. Cationic modifications, including, but not limited to, diethyl-ethylenediamide (DEED) or dimethyl-aminopropylamine (DMAP) may be especially useful due to decrease electrostatic repulsion between the oligonucleotide and a target. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

Examples of modified nucleotides with reduced charge include modified internucleotide linkages such as phosphate analogs having achiral and uncharged intersubunit linkages (e.g., Sterchak, E. P. et al., Organic. Chem., 52:4202, (1987)), and uncharged morpholino-based polymers having achiral intersubunit linkages (see, e.g., U.S. Pat. No. 5,034,506), as discussed above. Some internucleotide linkage analogs include morpholidate, acetal, and polyamide-linked heterocycles.

In another embodiment, the oligonucleotides are composed of locked nucleic acids. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

In some embodiments, the oligonucleotides are composed of peptide nucleic acids. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds. PNAs maintain spacing of heterocyclic bases that is similar to conventional DNA oligonucleotides, but are achiral and neutrally charged molecules. Peptide nucleic acids are composed of peptide nucleic acid monomers.

Other backbone modifications include peptide and amino acid variations and modifications. Thus, the backbone constituents of oligonucleotides such as PNA may be peptide linkages, or alternatively, they may be non-peptide peptide linkages. Examples include acetyl caps, amino spacers such as 8-amino-3,6-dioxaoctanoic acid (referred to herein as O-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Oligonucleotides optionally include one or more terminal residues or modifications at either or both termini to increase stability, and/or affinity of the oligonucleotide for its target. Commonly used positively charged moieties include the amino acids lysine and arginine, although other positively charged moieties may also be useful. Oligonucleotides may further be modified to be end capped to prevent degradation using a propylamine group. Procedures for 3′ or 5′ capping oligonucleotides are well known in the art.

In some embodiments, the nucleic acid can be single stranded or double stranded.

### D. Delivery Vehicles

The disclose compounds can be administered and taken up into the cells of a subject with or without the aid of a delivery vehicle. Appropriate delivery vehicles for the disclosed inhibitors are known in the art and can be selected to suit the particular inhibitor. For example, if the compound is a nucleic acid or vector, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome (see e.g., Pastan et al., (1988) Proc. Natl. Acad. Sci. U.S.A. 85:4486; Miller et al., (1986) Mol. Cell. Biol. 6:2895). The recombinant retrovirus can then be used to infect and thereby deliver to the infected cells nucleic acid encoding the compound inhibitor. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948 (1994)), adeno-associated viral (AAV) vectors (Goodman et al., Blood 84:1492-1500 (1994)), lentiviral vectors (Naidini et al., Science 272:263-267 (1996)), pseudotyped retroviral vectors (Agrawal et al., Exper. Hematol. 24:738-747 (1996)).

Physical transduction techniques can also be used, such as liposome delivery and receptor-mediated and other endocytosis mechanisms (see, for example, Schwartzenberger et al., Blood 87:472-478 (1996)). For example in some embodiments, the CTPS1 inhibitor is delivered via a liposome. Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.). This disclosed compositions and methods can be used in conjunction with any of these or other commonly used gene transfer methods.

In some embodiments, the delivery vehicle is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the compound. In some embodiments, release of the drug(s) is controlled by diffusion of the compound out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

### E. Protein Transduction Domains and Targeting Moieties

### 1. Protein Transduction Domains

Any of the compounds disclosed herein, and delivery vehicles including the compounds, can be modified to one or more domains for enhancing delivery of the compound across the plasma membrane in into the interior of cells. The compounds can be modified to include a protein transduction domain (PTD), also known as cell penetrating peptides (CPPS). PTDs are known in the art, and include, but are not limited to, small regions of proteins that are able to cross a cell membrane in a receptor-independent mechanism (Kabouridis, P., Trends in Biotechnology (11):498-503 (2003)). Although several of PTDs have been documented, the two most commonly employed PTDs are derived from TAT (Frankel and Pabo, Cell, 55(6):1189-93(1988)) protein of HIV and Antennapedia transcription factor from Drosophila, whose PTD is known as Penetratin (Derossi et al., J Biol Chem., 269(14):10444-50 (1994)).

The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of MV-1. The TAT PTD consists of an 11 amino acid sequence domain (residues 47 to 57; YGRKKRRQRRR (SEQ ID NO:16)) of the parent protein that appears to be important for uptake. Additionally, the basic domain Tat(49-57) or RKKRRQRRR (SEQ ID NO:17) has been shown to be a PTD. TAT has been favored for fusion to proteins of interest for cellular import. Several modifications to TAT, including substitutions of Glutatmine to Alanine, i.e., Q→A, have demonstrated an increase in cellular uptake anywhere from 90% (Wender et al., Proc Natl Acad Sci USA., 97(24):13003-8 (2000)) to up to 33 fold in mammalian cells. (Ho et al., Cancer Res., 61(2):474-7 (2001)) The most efficient uptake of modified proteins was revealed by mutagenesis experiments of TAT-PTD, showing that an 11 arginine stretch was several orders of magnitude more efficient as an intercellular delivery vehicle. Thus, some embodiments include PTDs that are cationic or amphipathic. Additionally, exemplary PTDs include, but are not limited to, poly-Arg—RRRRRRR (SEQ ID NO:18); PTD-5—RRQRRTSKLMKR (SEQ ID NO:19); Transportan GWTLNSAGYLLGKINLKALAALAKKIL (SEQ ID NO:20); KALA—WEAKLAKALAKALAKHLAKALAKALKCEA (SEQ ID NO:21); and RQIKIWFQNRRMIKWKK (SEQ ID NO:22).

In some embodiments, the compounds includes an endosomal escape sequence that improves delivery of the compound to the interior of the cell. Endosomal escape sequences are known in the art, see for example, Barka, et al., Histochem. Cytochem., 48(11):1453-60 (2000) and Wadia and Stan, Nat. Med., 10(3):310-5 (2004).

### 2. Targeting Signal or Domain

Any of the compounds disclosed herein and delivery vehicles including the compounds can be modified to include one or targeting signals or domains. The targeting signal or sequence can be specific for a host, tissue, organ, cell, organelle, an organelle such as the nucleus, or cellular compartment. Moreover, the compositions disclosed here can be targeted to other specific intercellular regions, compartments, or cell types.

In some embodiments, the targeting signal binds to a ligand or receptor which is located on the surface of a target cell such as to bring the compound and cell membranes sufficiently close to each other to allow penetration of the compound into the cell. Additional embodiments are directed to specifically delivering the compound to specific tissue or cell types.

Preferably, the targeting moiety enhances targeting to muscle, most preferably muscle satellite cells.

In a preferred embodiment, the targeting molecule is selected from the group consisting of an antibody or antigen binding fragment thereof, an antibody domain, an antigen, a cell surface receptor, a cell surface adhesion molecule, a viral envelope protein and a peptide selected by phage display that binds specifically to a defined cell.

Targeting domains to specific cells can be accomplished by modifying the disclosed compounds to include specific cell and tissue targeting signals. These sequences target specific cells and tissues, but in some embodiments the interaction of the targeting signal with the cell does not occur through a traditional receptor:ligand interaction. The eukaryotic cell includes a number of distinct cell surface molecules. The structure and function of each molecule can be specific to the origin, expression, character and structure of the cell. Determining the unique cell surface complement of molecules of a specific cell type can be determined using techniques well known in the art.

One skilled in the art will appreciate that the tropism of the compound can be altered by changing the targeting signal.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

Another embodiment provides an antibody or antigen binding fragment thereof bound to the disclosed recombinant polypeptides acting as the targeting signal. The antibodies or antigen binding fragment thereof are useful for directing the fusion protein to a cell type or cell state. In one embodiment, the fusion protein possesses an antibody binding domain, for example from proteins known to bind antibodies such as Protein A and Protein G from Staphylococcus aureus. Other domains known to bind antibodies are known in the art and can be substituted. In certain embodiments, the antibody is polyclonal, monoclonal, linear, humanized, chimeric or a fragment thereof. Representative antibody fragments are those fragments that bind the antibody binding portion of the non-viral vector and include Fab, Fab′, F(ab′), Fv diabodies, linear antibodies, single chain antibodies and bispecific antibodies known in the art.

In some embodiments, the targeting domain includes all or part of an antibody that directs the compound to the desired target cell type or cell state. Antibodies can be monoclonal or polyclonal, but are preferably monoclonal. For human gene therapy purposes, antibodies are derived from human genes and are specific for cell surface markers, and are produced to reduce potential immunogenicity to a human host as is known in the art. For example, transgenic mice which contain the entire human immunoglobulin gene cluster are capable of producing “human” antibodies can be utilized. In one embodiment, fragments of such human antibodies are employed as targeting signals. In a preferred embodiment, single chain antibodies modeled on human antibodies are prepared in prokaryotic culture.

Additional embodiments are directed to specifically delivering the compound to intracellular compartments or organelles. Eukaryotic cells contain membrane bound structures or organelles.

For example, in some embodiments, the compounds include a nuclear localization signal. Most proteins transported across the nuclear envelope contain a nuclear localization signal (NLS). The NLS is recognized by a nuclear import complex, enabling active transport to the nucleus. Even the transport of small proteins that can diffuse through the nuclear pore is increased by an NLS. NLS domains are known in the art and include for example, SV 40 T antigen or a fragment thereof, such as PKKKRKV (SEQ ID NO:23). The NLS can be simple cationic sequences of about 4 to about 8 amino acids, or can be bipartite having two interdependent positively charged clusters separated by a mutation resistant linker region of about 10-12 amino acids. The cauliflower mosaic virus (CMV) major capsid protein, CP, possesses an amino-terminal NLS. Additional representative NLS include but are not limited to GKKRSKV (SEQ ID NO:24); KSRKRKL (SEQ ID NO:25); KRPAATKKAGQAKKKKLDK (SEQ ID NO: 26); RKKRKTEEESPLKDKAKKSK (SEQ ID NO:27); KDCVMNKHHRNRCQYCRLQR (SEQ ID NO:28); PAAKRVKLD (SEQ ID NO:29); and KKYENVVIKRSPRKRGRPRK (SEQ ID NO:30).

### F. Formulations

The disclosed compounds can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are typically prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6^{th} Edition, Ansel et. al., (Media, PA: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric particles which provide controlled release of the active agent(s). In some embodiments, release of the drug(s) is controlled by diffusion of the active agent(s) out of the particles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing particles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

### 1. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants.

Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-. beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

### 3. Extended Release Dosage Forms

The extended release formulations are generally prepared as diffusion or osmotic systems, for example, as described in “Remington—The science and practice of pharmacy” (20th ed., Lippincott Williams & Wilkins, Baltimore, M D, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form having single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### 4. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods of Manufacturing

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material, typically although not necessarily incorporating a polymeric material, increasing drug particle size, placing the drug within a matrix, and forming complexes of the drug with a suitable complexing agent.

The delayed release dosage units may be coated with the delayed release polymer coating using conventional techniques, e.g., using a conventional coating pan, an airless spray technique, fluidized bed coating equipment (with or without a Wurster insert). For detailed information concerning materials, equipment and processes for preparing tablets and delayed release dosage forms, see Pharmaceutical Dosage Forms: Tablets, eds. Lieberman et al. (New York: Marcel Dekker, Inc., 1989), and Ansel et al., Pharmaceutical Dosage Forms and Drug Delivery Systems, 6.sup.th Ed. (Media, PA: Williams & Wilkins, 1995).

A preferred method for preparing extended release tablets is by compressing a drug-containing blend, e.g., blend of granules, prepared using a direct blend, wet-granulation, or dry-granulation process. Extended release tablets may also be molded rather than compressed, starting with a moist material containing a suitable water-soluble lubricant. However, tablets are preferably manufactured using compression rather than molding. A preferred method for forming extended release drug-containing blend is to mix drug particles directly with one or more excipients such as diluents (or fillers), binders, disintegrants, lubricants, glidants, and colorants. As an alternative to direct blending, a drug-containing blend may be prepared by using wet-granulation or dry-granulation processes. Beads containing the active agent may also be prepared by any one of a number of conventional techniques, typically starting from a fluid dispersion. For example, a typical method for preparing drug-containing beads involves dispersing or dissolving the active agent in a coating suspension or solution containing pharmaceutical excipients such as polyvinylpyrrolidone, methylcellulose, talc, metallic stearates, silicone dioxide, plasticizers or the like. The admixture is used to coat a bead core such as a sugar sphere (or so-called “non-pareil”) having a size of approximately 60 to 20 mesh.

An alternative procedure for preparing drug beads is by blending drug with one or more pharmaceutically acceptable excipients, such as microcrystalline cellulose, lactose, cellulose, polyvinyl pyrrolidone, talc, magnesium stearate, a disintegrant, etc., extruding the blend, spheronizing the extrudate, drying and optionally coating to form the immediate release beads.

### 5. Formulations for Mucosal and Pulmonary Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions composed of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

### 6. Topical and Transdermal Formulations

Transdermal formulations may also be prepared. These will typically be gels, ointments, lotions, sprays, or patches, all of which can be prepared using standard technology. Transdermal formulations can include penetration enhancers.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) composed of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

The basic difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components.

Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alkylene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Additional agents that can be added to the formulation include penetration enhancers. In some embodiments, the penetration enhancer increases the solubility of the drug, improves transdermal delivery of the drug across the skin, in particular across the stratum comeum, or a combination thereof. Some penetration enhancers cause dermal irritation, dermal toxicity and dermal allergies. However, the more commonly used ones include urea, (carbonyldiamide), imidurea, N, N-diethylformamide, N-methyl-2-pyrrolidone, 1-dodecal-azacyclopheptane-2-one, calcium thioglycate, 2-pyrrolidone, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.). Chemical penetrations and methods of increasing transdermal drug delivery are described in Inayat, et al., Tropical Journal of Pharmaceutical Research, 8(2):173-179 (2009) and Fox, et al., Molecules, 16:10507-10540 (2011). In some embodiments, the penetration enhancer is, or includes, an alcohol such ethanol, or others disclosed herein or known in the art.

Delivery of drugs by the transdermal route has been known for many years. Advantages of a transdermal drug delivery compared to other types of medication delivery such as oral, intravenous, intramuscular, etc., include avoidance of hepatic first pass metabolism, ability to discontinue administration by removal of the system, the ability to control drug delivery for a longer time than the usual gastrointestinal transit of oral dosage form, and the ability to modify the properties of the biological barrier to absorption.

Controlled release transdermal devices rely for their effect on delivery of a known flux of drug to the skin for a prolonged period of time, generally a day, several days, or a week. Two mechanisms are used to regulate the drug flux: either the drug is contained within a drug reservoir, which is separated from the skin of the wearer by a synthetic membrane, through which the drug diffuses; or the drug is held dissolved or suspended in a polymer matrix, through which the drug diffuses to the skin. Devices incorporating a reservoir will deliver a steady drug flux across the membrane as long as excess undissolved drug remains in the reservoir; matrix or monolithic devices are typically characterized by a falling drug flux with time, as the matrix layers closer to the skin are depleted of drug. Usually, reservoir patches include a porous membrane covering the reservoir of medication which can control release, while heat melting thin layers of medication embedded in the polymer matrix (e.g., the adhesive layer), can control release of drug from matrix or monolithic devices. Accordingly, the active agent can be released from a patch in a controlled fashion without necessarily being in a controlled release formulation.

Patches can include a liner which protects the patch during storage and is removed prior to use; drug or drug solution in direct contact with release liner; adhesive which serves to adhere the components of the patch together along with adhering the patch to the skin; one or more membranes, which can separate other layers, control the release of the drug from the reservoir and multi-layer patches, etc., and backing which protects the patch from the outer environment.

Common types of transdermal patches include, but are not limited to, single-layer drug-in-adhesive patches, wherein the adhesive layer contains the drug and serves to adhere the various layers of the patch together, along with the entire system to the skin, but is also responsible for the releasing of the drug; multi-layer drug-in-adhesive, wherein which is similar to a single-layer drug-in-adhesive patch, but contains multiple layers, for example, a layer for immediate release of the drug and another layer for control release of drug from the reservoir; reservoir patches wherein the drug layer is a liquid compartment containing a drug solution or suspension separated by the adhesive layer; matrix patches, wherein a drug layer of a semisolid matrix containing a drug solution or suspension which is surrounded and partially overlaid by the adhesive layer; and vapor patches, wherein an adhesive layer not only serves to adhere the various layers together but also to release vapor. Methods for making transdermal patches are described in U.S. Pat. Nos. 6,461,644, 6,676,961, 5,985,311, and 5,948,433.

### G. Combination Therapies

Combination therapies include administering a subject in need thereof a compositions that that reduces expression, activity, and/or bioavailability Pax6 and second therapeutic agent or other intervention. The second therapeutic can be a conventional therapeutic agent for treating a proteinopathy, amyloidosis, or a tauopathy. The second agent can determined based on the disease to be treated. For example, if the disease is Alzheimer's disease, the compositions disclosed herein can be coadministered with a conventional Alzheimer's disease treatment such as Aβ42 immunization (Wisniewski and Konietzko, Lancet Neurol., 7:805-811 (2008)), tarenflurbil (Flurizan™, Myriad Pharmaceuticals) which is believed to act by decreasing the production of Aβ42 (Aisen, Lancet Neurol., 7:468-469 (2008)), and tramiprosate (Alzhemed™, Neurochem Inc.) which was designed to bind to beta amyloid peptide and prevent it from reacting with glycosaminoglycans (Aisen et al., Curr. Alzheimer Res., 4:473-478 (2007)).

In some embodiments, the other intervention is or includes amyloid β removal.

### EXAMPLES

### Example 1: Pax6 is a Downstream Target for E2F1/c-Myb

### Materials and Methods

### Mining Human Brain Microarray Datasets

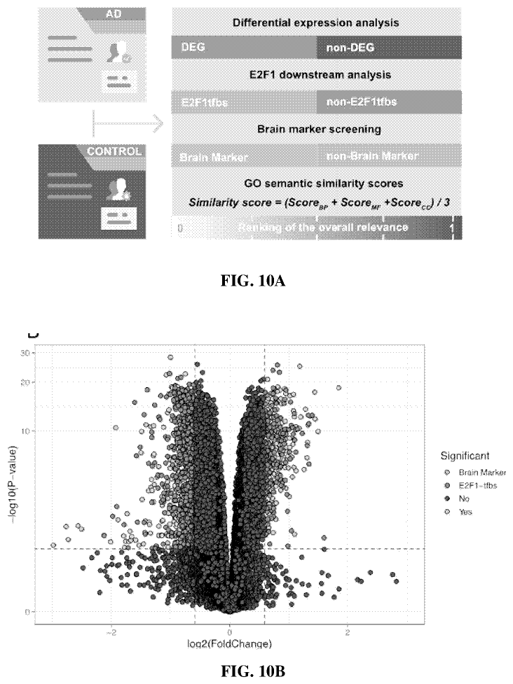

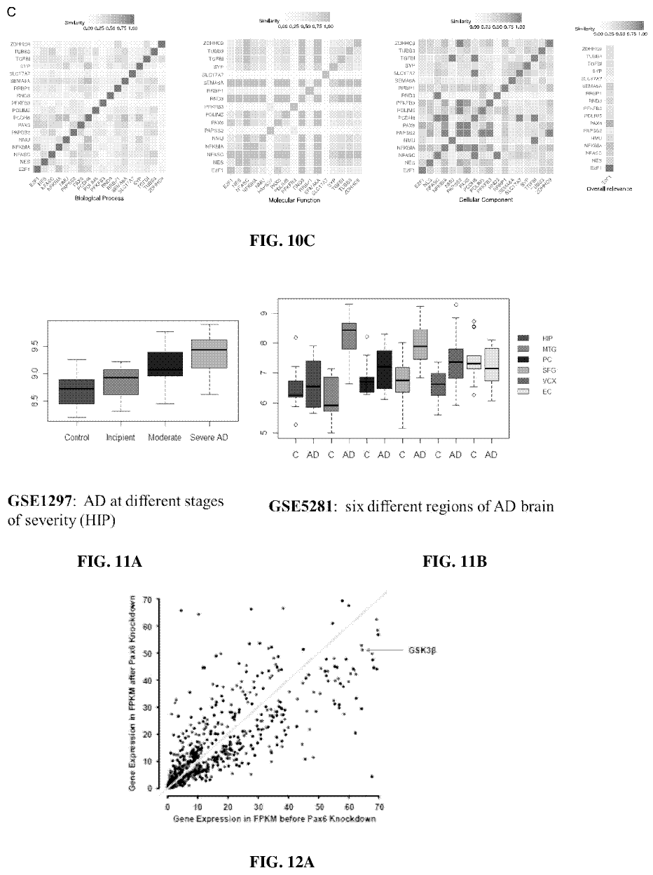

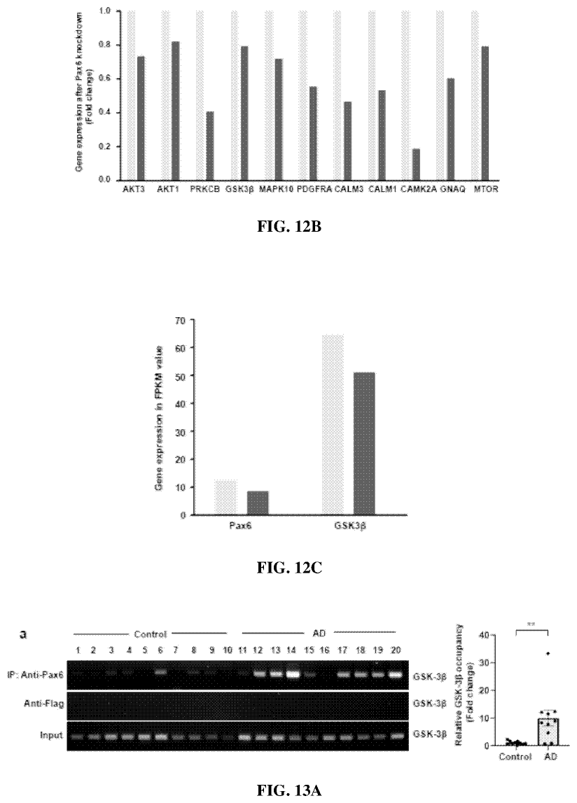

The workflow of data processing is shown in FIG. 10A. Four steps were performed to screen and rank genes regulated by E2F1. First, gene expression profile data for GSE15222²² were obtained from the Gene Expression Omnibus (GEO) database (platform GPL2700). Brain samples with a confirmed pathological diagnosis of late-onset Alzheimer's disease (n=176) and control brains (n=187) were extracted. Gene differential expression was assessed by R package Limma (version 3.40.6)²³, and genes with fold change>1.5 and adjusted p-value<0.05 were categorized as differentially expressed genes. ChIP-sequencing data sets for human transcription factor binding sites were downloaded for annotation and screening of the downstream targets of E2F1²⁴ Potential E2F1 downstream regulatory genes were derived by overlapping E2F1 transcription factor binding sites and the promoter regions (2 kb upstream to 1 kb downstream of the transcription start site for each gene) of the differentially expressed genes. Gene markers in the human brain were extracted from the Cell Marker database²⁵ for further screening. Finally, Gene Ontology (GO) semantic similarity scores of the selected genes were assessed by R package GOSemSim (version 2.10.0)²⁶. The overall relevance between E2F1 and the selected genes was calculated on the basis of the arithmetic average value of the three GO semantic similarity scores (biological process, molecular function and cellular component).

### Plasmid Constructs

Pax6 P1 promoter luciferase reporter construct pGL3b-Pax6 (1-346) was a gift from Dr. Andrew Chantry, Pax6 luciferase reporter construct P6CON-luc, PQ107 was a gift from Dr. Ales Cvekl, c-Myb shRNA construct pSiren-retroQ-Myb and control construct pSiren-retroQ-Luc were gifts from Dr. Robert K. Slany, c-Myb responsive reporter construct p5×MRE-A-luc (5×MRE) and wild type c-Myb construct pcDNA3.1-FL-c-Myb were gifts from Dr. Juraj Bies, E2F1 shRNA construct BS/U6 E2F1 RNAi and control construct BS/U6 RNAi vector were gifts from Dr. W. Douglas Cress, wild type E2F1 construct pcDNA3.1-HA-E2F1 was a gift from Dr. Joseph R. Nevins.

### Statistical Analysis

Unpaired two-tailed t-test and one-way ANOVA with Tukey's multiple comparison test were performed when groups were compared. P values were calculated using GraphPad Prism software version 8.0. All graphs show means SEM. Differences were considered significant at *p<0.05.

### Results

To examine the downstream events in the Cdk4/pRb/E2F1 pathway, workflow of data processing is shown in FIG. 10A and Methods. In an array-based expression analysis (GSE15222)²², of the 1389 genes identified as differentially expressed between the 176 Alzheimer's disease brains and 187 healthy brains, 670 genes were predicted to be E2F1 regulatory targets from a chromatin immunoprecipitation (ChIP)-sequencing dataset. Among them, 16 genes were confirmed as brain cell markers²⁵ (FIG. 10B). Using semantic similarity among three Gene Ontology (GO) terms (biological process [BP], molecular function [MF] and cellular component [CC]) of the 16 genes, Pax6 showed the highest overall correlation with E2F1 (FIG. 10C) and was significantly upregulated in human Alzheimer's disease brains.

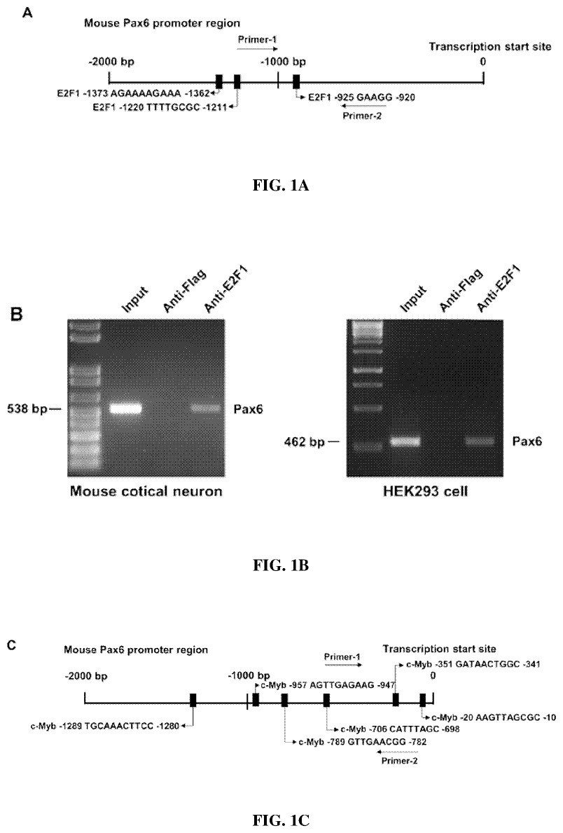

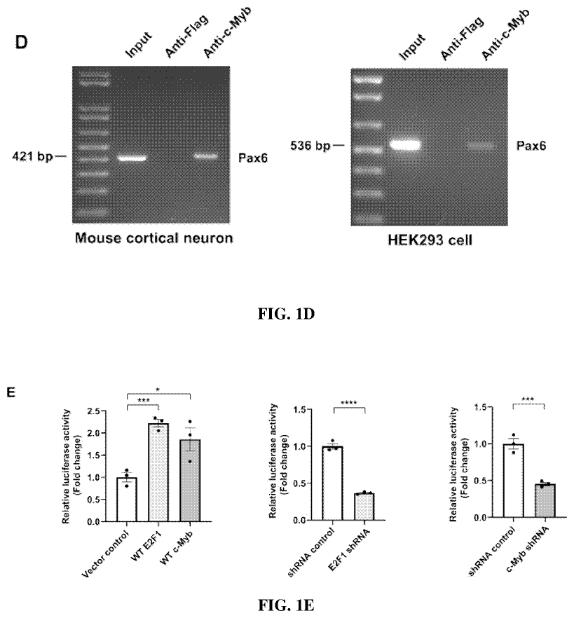

Upon further analysis, transcription factor binding sites were identified in the promoters of several novel downstream targets (e.g. Pax6 and c-Myb) of E2F1. Although Pax6 is a known transcription factor important for eye, brain and olfactory system development³⁴⁻³⁶, there is no information about Pax6 involvement in the E2F1 pathway and its function in Alzheimer's disease pathogenesis. Both the human and murine Pax6 promoters harbored putative E2F1 and c-Myb binding sites situated closely together (FIGS. 1A and C). ChIP analyses was performed for E2F1 and c-Myb in murine cortical neurons and human HEK293 cells and found that both the mouse and human Pax6 promoters were occupied by E2F1 and c-Myb (FIGS. 1B and 1D). To investigate whether E2F1 and c-Myb transactivate Pax6 in vitro, both wild-type E2F1 and c-Myb were overexpressed in murine neuroblastoma cells (N2a). Increased Pax6 promoter activity in a luciferase promoter assay were observed (FIG. 1E). By contrast, shRNA-mediated knockdown of E2F1 or c-Myb resulted in decreased Pax6 promoter activity in N2a cells (FIG. 1E). These data confirmed that both E2F1 and c-Myb transactivate Pax6.

### Example 2: Pax6 and c-Myb are Overexpressed in Alzheimer's Disease Post-Mortem Brains

### Materials and Methods

### Human Brain Specimens and Animals

Post-mortem human brain tissues from the frontal cortex of patients with Alzheimer's disease and nondemented control subjects were obtained from the Canadian Brain Tissue Bank and New York Brain Bank of Columbia University. Study of human brain tissue was approved by the Ethics Committee of the Faculty of Medicine of the University of Toronto (NO: 00026798) and the University of Hong Kong (NO: UW 13-177). All mice were on a C57BL/6 genetic background. TgCRND8 mice express human amyloid precursor protein 695 (APP695) with the Swedish mutation (KM670/671NL) and Indiana mutation (V717F) under the control of the PrP gene promoter²⁰. All experiments using animals were approved by the Committee for the Use of Live Animals in Teaching and Research at the University of Hong Kong (NO: CULATR2792-12, CULATR 3732-15, CULATR 5212-19).

### Antibodies and Reagents

For western blot, anti-Pax6 (Thermo Fisher Scientific, 42-6600, 1:1000), anti-Pax6 (Sigma, SAB5300039, 1:1000), anti-c-Myb (Cell Signaling, 12319, 1:1000), anti-c-Myb (Abcam, ab117635, 1:3000), anti-E2F1 (Santa Cruz, sc-251, 1:1000), anti-GSK-3β (Cell Signaling, 9315, 1:3000), anti-phospho-Tau (pSer356) (Sigma, SAB4504556, 1:1000), anti-phospho-Tau (pSer396) (Sigma, T7319, 1:3000), anti-phospho-Tau (pSer404) (Sigma, T7444, 1:3000), anti-β-actin (Cell Signaling, 4970, 1:3000), anti-GAPDH (Cell Signaling, 2118, 1:3000). For chromatin immunoprecipitation, anti-Pax6 (Santa Cruz, sc-32766, 1:50), anti-c-Myb (Santa Cruz, sc-74512, 1:50), anti-E2F1 (Santa Cruz, sc-251, 1:50), anti-Flag (Sigma, F1804, 1:500). For immunostaining, anti-Pax6 (Covance, PRB-278P, 1:2000), anti-NeuN (Chemicon, MAB377, 1:1000), secondary antibodies goat anti-rabbit Alexa Fluor 568 (Thermo Fisher Scientific, A-11011, 1:4000) and goat anti-mouse Alexa Fluor 488 (Thermo Fisher Scientific, A-11008, 1:4000). For primary neuron treatment, amyloid β₁₋₄₂ peptide was purchased from Bachem (H1368), flavopiridol was purchased from Sigma (F3055).

### Western Blot Analysis

Human brain tissues or mouse cortical neurons were dissolved in ice-cold 1×RIPA buffer (Cell Signaling) with proteinase inhibitor cocktail (Sigma). Proteins were separated on NuPAGE Novex 4-12% Bis-Tris gels (Thermo Fisher Scientific) and transferred to nitrocellulose membranes. Membranes were probed with indicated primary antibodies. The blots were analysed and quantified by densitometry using ImageJ software (Version 1.52t, National Institutes of Health).

### Results

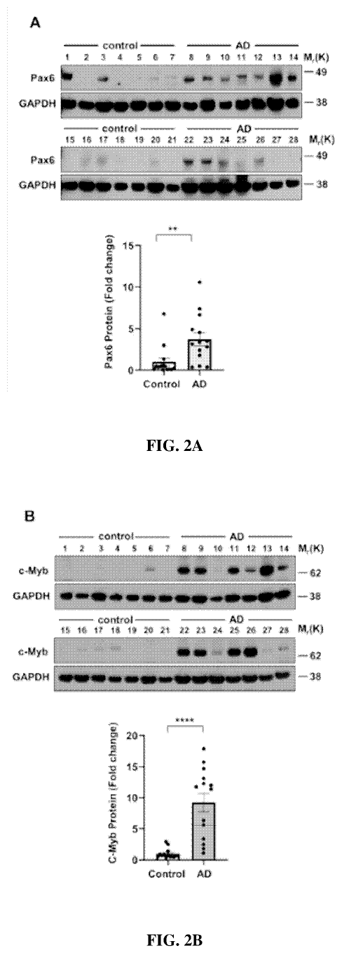

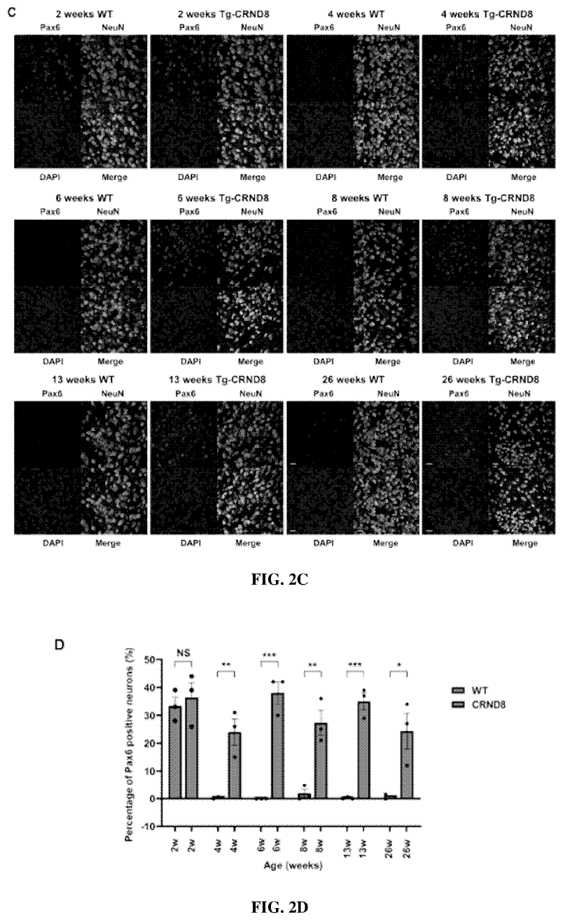

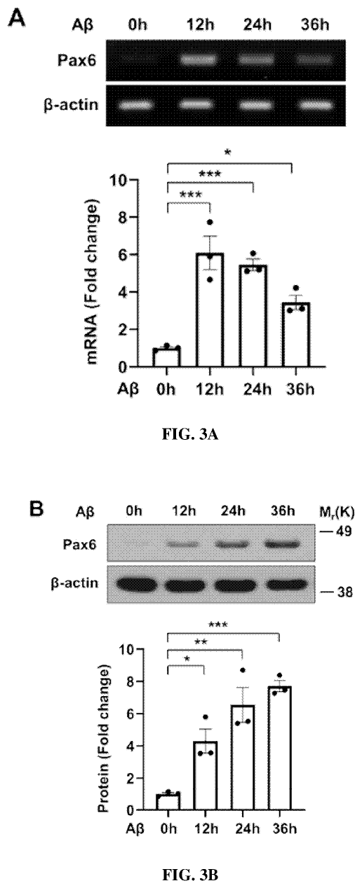

The expression profile of Pax6 and c-Myb were investigated in an independent set of human brain tissues (Table 3, below). Western blot of frontal cortical tissue isolated from 14 Alzheimer's disease patients and 14 non-Alzheimer's disease controls showed that expression of Pax6 (FIG. 2A, P=0.0066) and c-Myb (FIG. 2B, P<0.001) was significantly upregulated in Alzheimer's disease brains compared with controls (FIGS. 2A and 2B).

### Example 3: Pax6 is Increased in the Entorhinal Cortex of APP Transgenic Mice

### Materials and Methods

### Immunostaining

TgCRND8 mice and non-Tg littermate pairs at 2, 4, 6, 8, 13, 26 weeks of age were sacrificed by transcardial perfusion with 1×PBS, brains were fixed in 4% paraformaldehyde and cryoprotected. Horizontal sections were cryostat-cut at the thickness of ten microns from OCT embedded frozen blocks and mounted onto gelatin-coated slides. The sections were blocked with 10% goat serum (Sigma) in antibody diluent (Dako) for one hour at room temperature. The sections were incubated overnight at 4° C. with: Rabbit anti-Pax6 (Covance, 1:2000) and mouse anti-NeuN (Chemicon, 1:1000) antibodies (prepared in antibody diluent (Dako)), followed by incubation with second antibodies goat anti-mouse antibodies (Alexa Fluor 488, Thermo Fisher Scientific, 1:4000) or goat anti-rabbit antibodies (Alexa Fluor 568, Thermo Fisher Scientific, 1:4000) for one hour. The slides were mounted in a DAPI/Antifade mounting medium (Vectashield). Fluorescent images were captured using a confocal laser-scanning microscope (LSM 710, Carl Zeiss).

### Results

To determine if Pax6 is also induced in an in vivo Alzheimer's disease model, experiments were designed to examine whether Pax6 protein is increased in TgCRND8 mice, which overproduce toxic amyloid β₁₋₄₂²⁰. The entorhinal cortex is the first brain area to be affected in Alzheimer's disease and its atrophy is highly associated with episodic memory impairment in Alzheimer's disease patients^{37, 38}. Immunohistochemical staining of TgCRND8 and wild-type littermate mouse brains showed that beginning at 4 weeks of age, the proportion of neurons expressing Pax6 was significantly increased in the entorhinal cortex of TgCRND8 mice (FIG. 2C). This increase lasts until the mice are 26 weeks old. Pax6 was localised exclusively in neurons and inside the nucleus (FIG. 2C).

### Example 4: Pax6 and c-Myb are Increased in Response to Amyloid β Toxicity

### Materials and Methods

### Neuronal Survival Assay

The survival assay of amyloid β neurotoxicity was performed as reported previously³³. At various time periods, neurons were lysed in a cell lysis buffer. Under light microscopy, the numbers of intact nuclei indicating living cells was quantified and expressed relative to the number of cells in the control group without amyloid β treatment.

### Semi-Quantitative RT-PCR

Total mRNA was prepared from cell cultures utilizing TRIzol reagent (Thermo Fisher Scientific). Semi-quantitative RT-PCR experiments were performed to examine RNA expression of target genes. Briefly, 2 μg of total RNA was reverse-transcribed using SuperScript First-Strand Synthesis System (Thermo Fisher Scientific). The first-strand cDNA was used as a template. β-actin was used as the normalisation control.

### Amyloid β Preparation

Oligomeric amyloid β₁₋₄₂ peptide (Bachem) was prepared as described previously³². Briefly, lyophilised, HPLC-purified amyloid β₁₋₄₂ was equilibrated and reconstituted in 100% 1,1,1,3,3,3 hexafluoro-2-propanol (HFIP; Sigma) to 1 mM. HFIP was evaporated and the crystallised peptide was air-dried and was reconstituted to 5 mM in dimethyl sulfoxide (DMSO) followed by sonication. The 5 mM amyloid β₁₋₄₂ stock solution was then diluted with phosphate buffered saline (PBS) to 400 μM and was incubated for 18-24 h at 37° C. The solution was diluted again for a working concentration of 100 μM. The resulting solution was further incubated at 37° C. for 18-24 hours and the amyloid β peptide is ready for use.

### Luciferase Reporter Assay

Cortical neurons were transfected with luciferase reporter constructs three days after plating. After amyloid β treatment, luciferase activity of a target gene was determined using the dual-luciferase reporter assay system (Promega) in a microplate luminometer. Luciferase activity was normalised against Renilla activity for the transfection efficiency using a pRL Renilla luciferase control reporter construct.

### Results

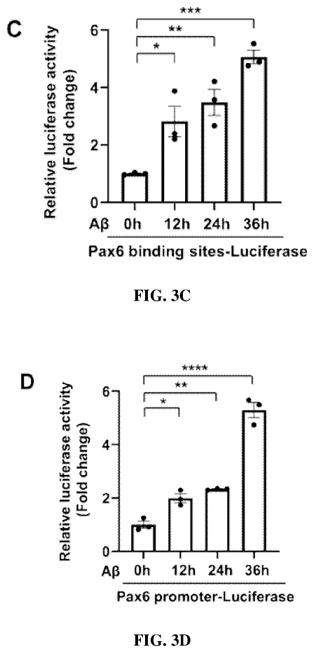

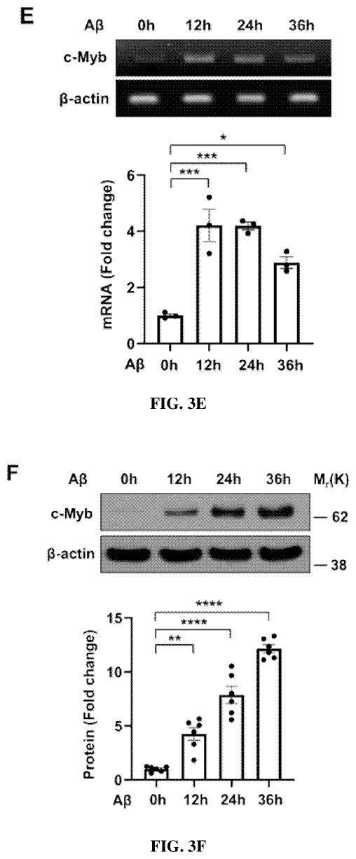

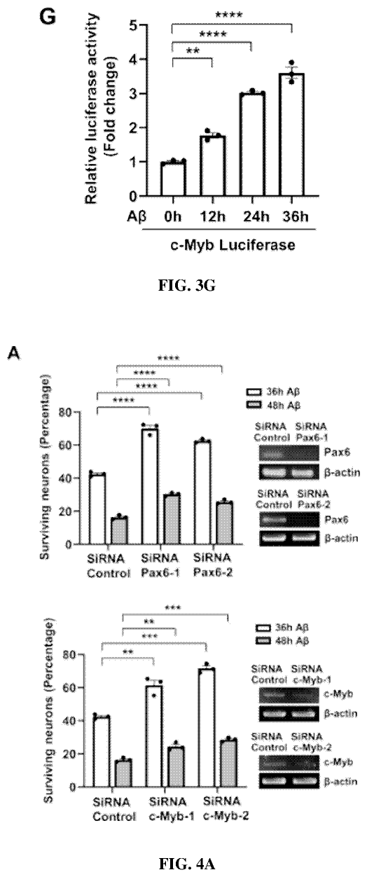

To study in detail the molecular mechanism of the Cdk/pRb/E2F1 pathway, experiments were designed to examine whether Pax6 or c-Myb expression changed in cultured cortical neurons after amyloid β treatment. Neurons were exposed to oligomeric amyloid β₁₋₄₂ (5 μM) for 12, 24 or 36 h, and analyzed for PAX6 and c-MYB mRNA levels by RT-PCR. Results showed that the expression of both genes were significantly increased after amyloid β exposure, and both transcripts peaked following a 12 h treatment (FIGS. 3A and 3E). Consistent with the transcriptional upregulation, Western blot analysis showed that both the Pax6 and c-Myb proteins increased in a similar manner (FIGS. 3B and 3F). Notably, the basal endogenous mRNA and protein amounts of both genes were almost undetectable in postmitotic neurons, indicating that Pax6 and c-Myb might be functionally quiescent without stress induction.

Next, experiments were designed to investigate whether the upregulation of Pax6 and c-Myb is associated with an increase in transcriptional activation of their targets. Cortical neurons were transfected with a P6CON luciferase reporter construct, which contained three standard Pax6 binding sites³⁹ to measure Pax6 binding; a luciferase construct containing the Pax6 P1 promoter⁴⁰ to measure Pax6 promoter activation; or with a luciferase construct containing c-Myb binding sites to measure c-Myb binding. The same luciferase assays were then performed after amyloid β treatment. Results showed that the amyloid β challenge induced Pax6 DNA-binding activity (FIG. 3C), Pax6 promoter activity (FIG. 3D) and c-Myb binding (FIG. 3G). Taken together, these data indicate that amyloid β-induced increase in Pax6 and c-Myb mRNA and protein levels correlate well with increased transcriptional activity of Pax6 and c-Myb in Alzheimer's disease, indicating that Pax6 and c-Myb might have a role in neuronal apoptosis.

### Example 5: Pax6 and c-Myb are Amyloid β-Induced Proapoptotic Proteins

### Materials and Methods

### Primary Neuronal Cultures and RNA Interference

Primary cortical neurons were derived from embryonic day 14.5 (E14.5) C57BL/6 mice³¹. The brains were dissociated from skulls, meninges removed and cortices isolated. Individual cells were dissociated by incubation with trypsin and DNase (Sigma). Cells were plated on poly-d-lysine (Sigma)-coated 24-well plates at a density of 1.5×10⁶ cells/ml in Neurobasal medium (Thermo Fisher Scientific) supplemented with B-27 (Thermo Fisher Scientific), N-2 (Thermo Fisher Scientific), and glutamine (0.5 mM, Thermo Fisher Scientific). Three days after seeding, cultured cells were transfected with siRNAs (60 pmol/24-well) using Lipofactamine 2000 transfection reagent (Thermo Fisher Scientific). Twenty-four hours after transfection, cells were exposed to amyloid β₁₋₄₂ (5 μM) for the indicated time periods and then processed for subsequent assays. SiRNAs from Santa Cruz are provided as pools consisting of three to five target-specific 19-25 nucleotides siRNAs designed to specifically knockdown the expression of mouse Pax6, c-Myb or E2F1. Silencer Select Pre-designed siRNAs were from Ambion, non-targeting siRNA was used as a negative control. Knockdown efficiency per target was tested by RT-PCR or Western blot.

### Results

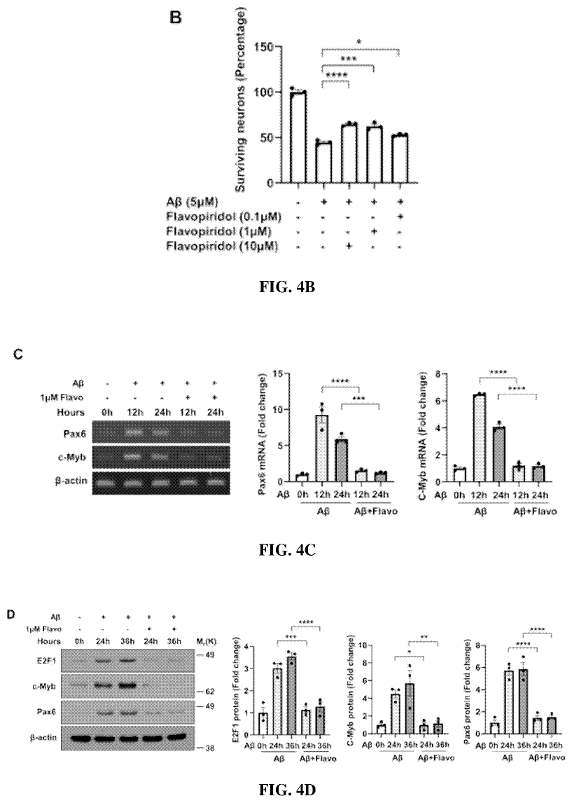

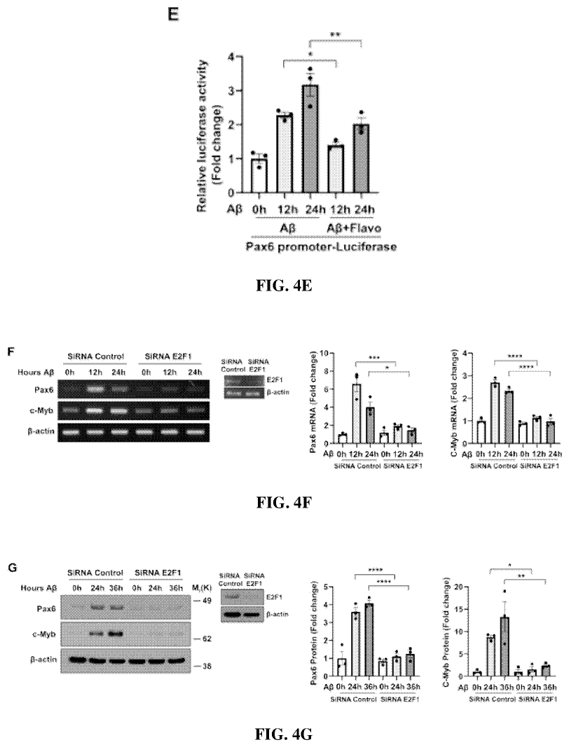

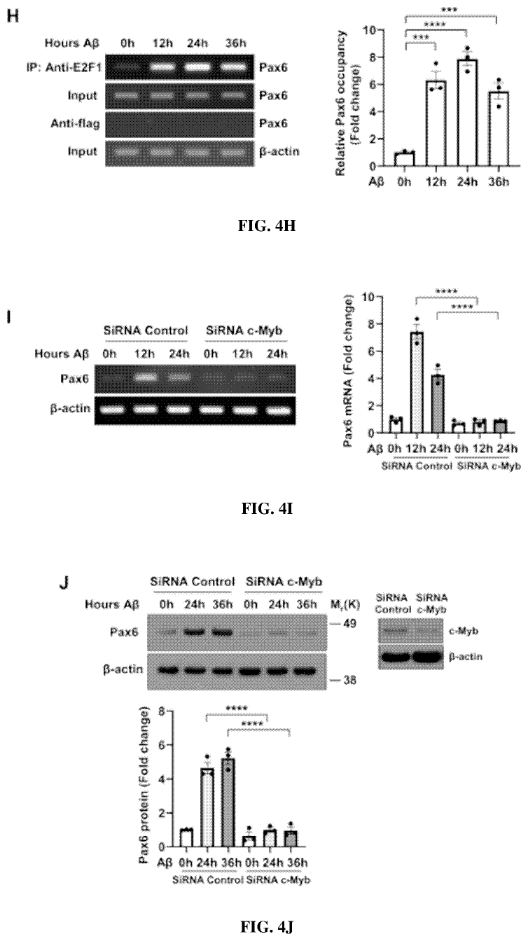

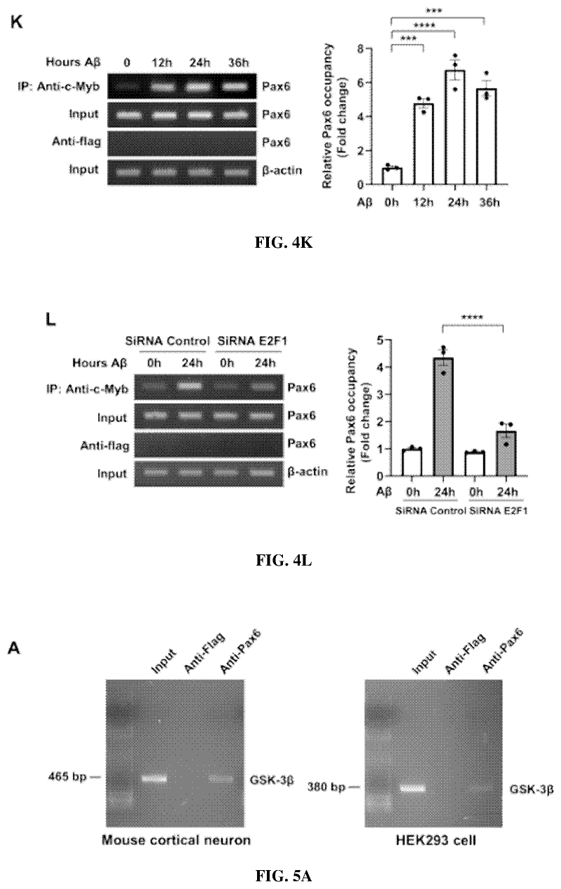

Experiments were designed to examine Pax6 and c-Myb function for neuronal apoptosis. Cortical neurons were transfected with Pax6 or c-Myb siRNA oligonucleotides and treated with amyloid β₁₋₄₂ oligomers (5 μM), and a survival assay was performed. Results showed that two Pax6-specific and two c-Myb-specific siRNA oligonucleotides offered significant protection from amyloid β₁₋₄₂ treatment compared with a control siRNA (about 40% survival with control siRNA vs 70% with Pax6 knockdown or 60% survival with c-Myb knockdown; FIG. 4A). These data indicate that Pax6 and c-Myb are potential mediators of amyloid β neurotoxicity.

### Example 6: Cdk Activity is Required for Amyloid β-Induced c-Myb and Pax6 Activation

To test the hypothesis that the Cdk/pRb/E2F1 pathway acts upstream of c-Myb and Pax6, a potent Cdk inhibitor, flavopiridol, was utilized. Because multiple Cdks could potentially modulate apoptotic signals, the effect of flavoporidol on neuronal survival was tested. Flavoporidol protected cortical neurons against amyloid β-induced death (FIG. 4B) and significantly blocked amyloid β-induced upregulation of Pax6 and c-Myb mRNA and protein (FIGS. 4C and 4D). Notably, co-treatment with flavopiridol blocked amyloid β-induced activation of the Pax6 promoter (FIG. 4E). These findings indicate that Cdk activity is important for the upregulation and activation of both transcription factors.

### Example 7: E2F1 and c-Myb Synergistically Transactivate Pax6 in Amyloid β-Induced Toxicity

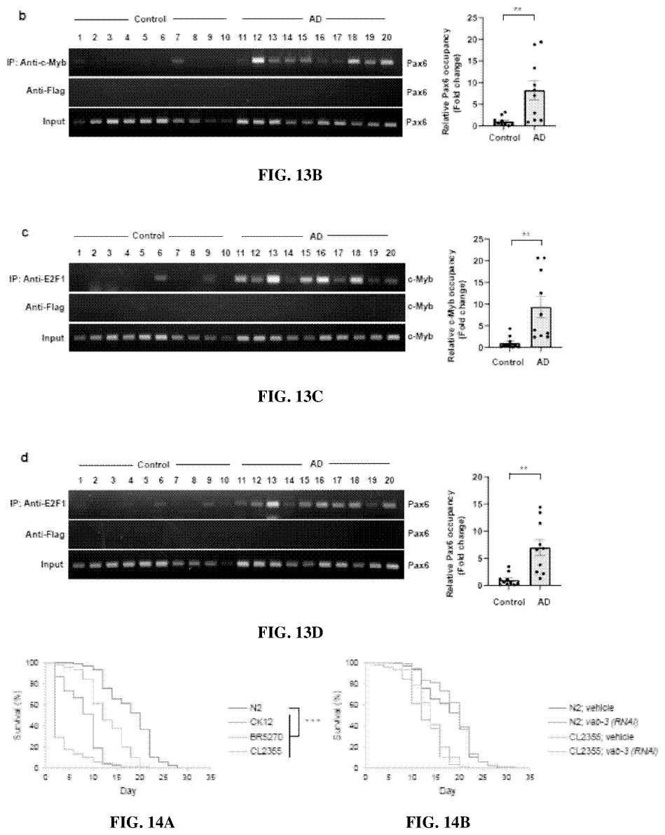

Next, experiments were designed to investigate whether E2F1 acts upstream of c-Myb and Pax6 to mediate amyloid β toxicity. Using siRNA to downregulate E2F1 expression, results showed that E2F1 silencing significantly blocked the upregulation of Pax6 and c-Myb mRNA and protein (FIG. 4F, 4G). Binding of E2F1 to the Pax6 promoter was significantly increased after amyloid β treatment (FIG. 4H). The effect of E2F1 and c-Myb on Pax6 promoter binding and E2F1 on c-Myb promoter binding was also investigated in post-mortem Alzheimer's disease brain. Chromatin immunoprecipitation of 10 Alzheimer's disease and 10 control brains showed that all three binding affinities increased in Alzheimer's disease frontal cortex tissue compared with control frontal cortex tissue (FIGS. 13B-13D). Taken together, these findings indicate that E2F1 is responsible for the amyloid β-induced c-Myb and Pax6 increase.

Furthermore, c-Myb downregulation decreased Pax6 expression at the mRNA and protein levels (FIGS. 4I and 4J). Additionally, amyloid β treatment of neurons substantially increased occupancy of the Pax6 promoter by c-Myb (FIG. 4K). Importantly, this increase can be blocked by E2F1 silencing (FIG. 4L), indicating an amyloid β-induced signaling response in which E2F1 can either directly activate Pax6 at the transcriptional level or transactivate Pax6 through c-Myb activation.

### Example 8: Pax6 Transactivates GSK-3β to Promote Phosphorylation of Tau Protein

### Materials and Methods

### RNA-Sequencing

Total mRNA was extracted from cultured mouse primary neurons transfected with a control siRNA and Pax6 siRNA. For each control siRNA or Pax6 siRNA treatment, one biological replicate was prepared. All samples were sequenced by Axeq using Illumina HiSeq 2×100 bp paired-end sequencing. Eight raw sequence files were generated in FASTQ format. The quality of RNA-sequencing raw reads was evaluated using NGS QC Toolkit (version 2.2.3). The total number of reads per sequence file was around 32 million and the average read length was 101 bp. The overall PHRED quality scores for all sequence files was higher than 20 and the peak value of GC content distribution for each sequence file was 45-55%. No further trimming or filtering was applied to the raw data.

### Mapping RNA-Sequencing Reads to a Mouse Reference Genome Using TopHat

Millions of short reads from RNA-sequencing were aligned to the mouse reference genome using TopHat (version 2.0.3). TopHat first applies an unspliced aligner, Bowtie, to align exon reads to a reference genome without considering any large gaps and then deals with the small portion of unmapped reads at splicing junctions²⁷. The mouse reference genome sequence was downloaded from the TopHat website (Mus musculus iGenome Ensembl NCBIM37). TopHat accepts raw reads files in FASTQ format as input and outputs the alignment results in BAM format. Default parameters were used to run TopHat. Over 80% of the reads could be mapped to the reference genome.

### Aligned Reads Summarization and Transcriptome Reconstruction Using Cufflinks

Aligned RNA read files in BAM format were inputted into Cufflinks (version 2.0.0) to assemble the aligned reads into transcripts. Cufflinks uses a reference genome-guided method to assemble exons, identify novel transcripts and report a minimal set of isoforms to best describe the reads in the dataset²⁸. Cufflinks normalises the raw fragment counts with a maximum likelihood estimation method and quantifies the expression of transcripts as a Fragments Per Kilobase of exon model per Million mapped fragments (FPKM) value. Cuffmerge was used to merge all transcript assemblies in GTF format to construct a more complete and accurate transcriptome. This transcriptome was then compared with the mouse reference annotation file downloaded from Ensembl (NCBIM37) to identify novel genes and transcripts by Cuffcompare²⁹.

### Differential Expression Test Using Cuffdiff and Candidate Gene Selection

Cuffdiff, which is included in the Cufflinks package, was used to detect differentially expressed genes or transcripts between the normal control and the Pax6 knockdown groups in the mouse RNA-sequencing experiment. Differential gene expression after Pax6 knockdown was calculated by the ratio of FPKM value at P0 versus C0. In total, 8584 genes (4520 downregulated and 4064 upregulated) were identified as differentially expressed with FDR-adjusted p-values lower than 0.05. Pathway enrichment analysis based on these differentially expressed genes identified 60 enriched Kyoto Encyclopedia of Genes and Genomes (KEGG) pathways with p-values lower than 10⁴. Alzheimer's disease-related KEGG pathways were defined as pathways that share at least one gene with the Alzheimer's disease pathway hsa05010; 77 Alzheimer's disease-related KEGG pathways were identified. As a result, 34 Alzheimer's disease-related enriched KEGG pathways were chosen and genes involved in these pathways were downloaded from the KEGG database³⁰. We searched the promoter regions of all differentially expressed geneses for Pax6 binding sites using a Pax6 binding matrix from JASPAR. Finally, 33 genes were selected that met all of the following criteria: more than 1.2 expression fold change, predicted to have Pax6 binding sites and involved in at least three Alzheimer's disease-related enriched KEGG pathways (Table 1, below).

### Chromatin Immunoprecipitation (Chip)

ChIP experiments were conducted as previously described²¹. Mouse cortical neurons and HEK293 cells were harvested and cell lysates were sonicated and centrifuged, and the supernatants collected for immunoprecipitation. Brain samples from 10 Alzheimer's disease cases and 10 controls were obtained from the University of Columbia. Equal amounts (30-40 mg) of frozen tissue from each sample were weighed and thawed on ice. Tissues were chopped with a scalpel and transferred to conical tubes with ice-cold PBS containing protease inhibitor cocktail (Sigma). Tissues were cross-linked with 1% formaldehyde in PBS for 10 min at room temperature and 125 mM glycine was added to quench the reaction. Tissues were centrifuged at low speed (1300 rpm) at 4° C. for 5 min and the supernatant was removed. The pellets were washed with ice-cold PBS twice, centrifuged and resuspended in lysis buffer (1% SDS, 10 mM EDTA, 50 mM Tris/HCl, pH 8.1) containing protease inhibitor cocktail (Sigma). They were homogenised with a Dounce tissue grinder pestle in 20 strokes, and lysates were sheared by sonication for 50-100 s in 10 s bursts. Samples were centrifuged at 4° C. for 10 min at 15000 g to remove debris, and supernatants were diluted 1:10 in a lysis buffer (0.01% SDS, 1% Triton X-100, 2 mM EDTA, 20 mM Tris/HCl, pH 8.1, 150 mM NaCl). Aliquot of 20 μl from each undiluted sample was stored for the input chromatin. Immunoprecipitations were performed with specific antibodies at 4° C. overnight with rotation, a non-specific anti-Flag antibody was used as a negative control. 30 μl of ChIP-Grade Protein G Magnetic Beads slurry (#9006, Cell Signalling) was added to each sample and then incubated with rotation for 2 h. Beads were pelleted using a magnetic separation rack and washed four times with washing buffer. Beads were eluted twice with elution buffer (1% SDS, 0.1 M NaHCO₃) by rotation at 30° C. for 15 minutes. Formaldehyde cross-linking was reversed by overnight heating at 65° C., as well as input, incubating for 1 hour with RNaseA at 37° C. (20 μg/ml) and then for 1 h with proteinase K at 42° C. (20 μg/ml). DNA was purified using a PCR purification kit (Qiagen), and samples were analysed by semi-quantitative PCR.

### Results

To identify downstream target genes for Pax6, a comparative gene expression analysis was performed using RNA sequencing. mRNA samples from murine cortical neurons transfected with control or Pax6 siRNA were analyzed. Significant differences in the expression of many Alzheimer's disease-related genes between the Pax6-silenced and control groups (Table 1, below) were observed. For example, regulator of G-protein signaling 14 (RGS14) was most downregulated; RGS14 is reported to be a key regulator of signaling pathways linking synaptic plasticity in CA2 pyramidal neurons to hippocampal-based learning and memory⁴¹. Specifically, results showed found that Pax6 transcriptionally regulated multiple major kinases that phosphorylate tau, including Cdk5 and p35, GSK-3β, mitogen-activated protein kinases (MAPK), serine/threonine protein kinase (MARK), calcium/calmodulin-dependent protein kinase type II a (CAMK2a), etc (Table 2, below).

In these experiments, focus was directed to validating GSK-3β, which might be a direct target of Pax6. GSK-3β is a proline-directed serine-threonine kinase that has been postulated to have a role in tau phosphorylation and neurofibrillary tangle formation⁴². In the RNA-sequencing screening for Pax6 target genes, results showed that a 40.0% downregulation of Pax6 mRNA causes a 33.5% reduction in GSK-3β mRNA expression. There were two Pax6 binding sites in the GSK-3β promoter sequence in both mice and humans.

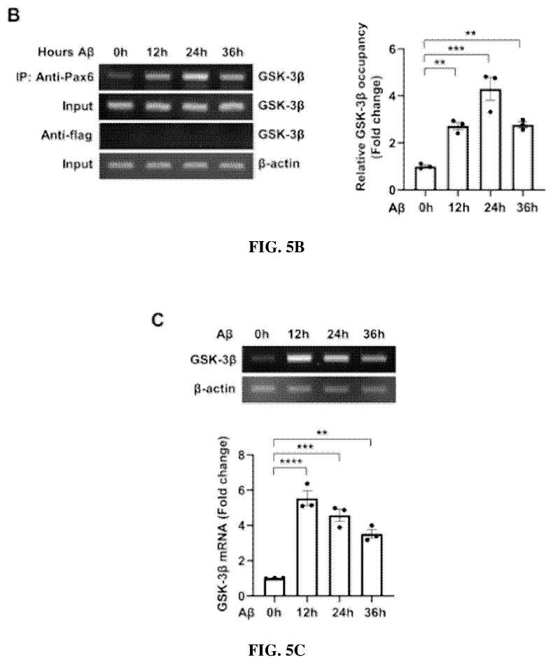

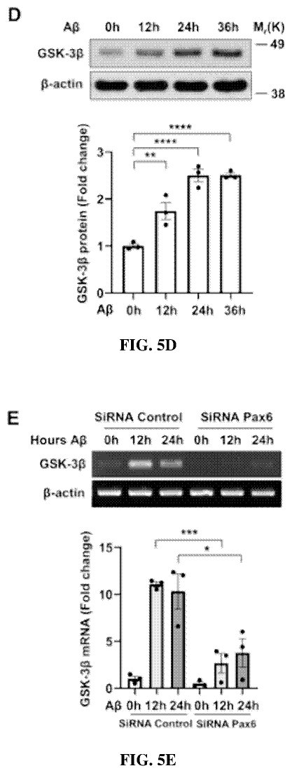

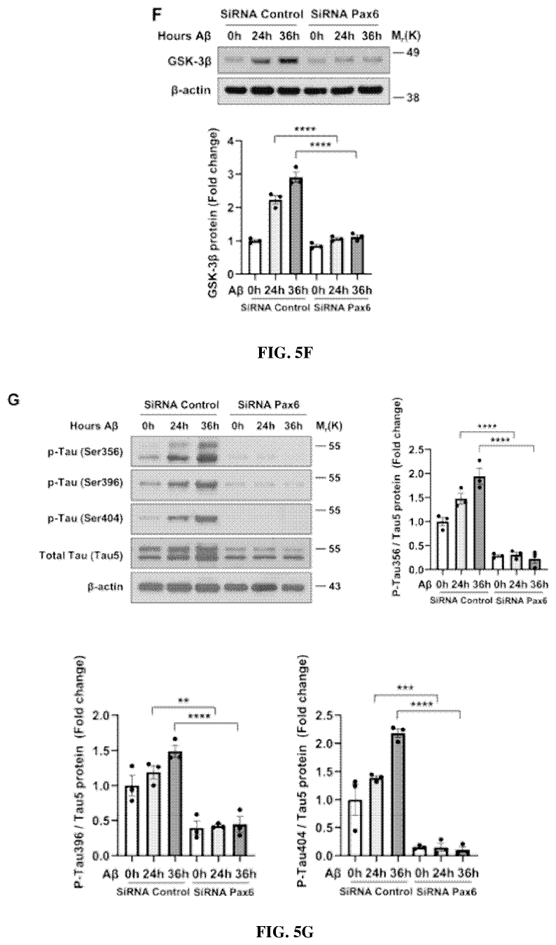

Next Pax6 and GSK-3β interaction was examined by ChIP assay in untreated HEK293 cells and murine cortical neurons and results showed that the GSK-3β promoter region was occupied by Pax6 in both species (FIG. 5A). The responsiveness of the GSK-3β promoter to Pax6 was tested in an amyloid β model in mouse cortical neurons. Amyloid β treatment significantly increased Pax6 occupancy of the GSK-3β promoter (FIG. 5B). This increased binding affinity was also observed in human Alzheimer's disease brain compared with non-Alzheimer's disease controls (FIG. 13A). Amyloid β treatment increased GSK-3β mRNA and protein levels (FIGS. 5C and 5D), which was blocked by siRNA-mediated Pax6 knockdown (FIGS. 5E and 5F). These data indicate that GSK-3β acts as a downstream effector of Pax6 in amyloid β signaling.

Hyperphosphorylation of tau at serine and threonine residues is a hallmark of neurofibrillary tangles in Alzheimer's disease^{43, 44}. Since GSK-3β kinase is involved in regulating tau phosphorylation, we reasoned that Pax6 induction might also regulate tau phosphorylation in our amyloid β toxicity paradigm. To test this idea, we downregulated Pax6 with siRNA and examined tau phosphorylation sites. Amyloid β challenge increased the concentration of phospho-tau Ser 356, 396 and 404 (FIG. 5G). When Pax6 was downregulated, tau phosphorylation at the three serine sites and the ratio of phospho-tau to total tau were strongly decreased, suggesting that the cell cycle pathway mediates GSK-3β activity in this paradigm. This result was consistent with a previous report that aggregated amyloid β can activate GSK-3β to phosphorylate tau⁴⁵.

### Example 9: Reduced Pax6 Expression Improves Learning and Memory Ability of AD Mice

### Materials and Methods

For directly inhibition of Pax6 to reduce total tau and p-tau in Alzheimer using nucleic acids, we have performed below:
1) Knockdown of Pax6 using siRNA in mouse primary neurons and injected AAV Pax6 shRNA at hippocampus in mice:

Sequence 1:
(SEQ ID NO: 31)
5′-CCACTTCAACAGGACTCATTT-3′
Sequence 2:
(SEQ ID NO: 32)
5′-GCAAGAATACAGGTATGGTTT-3′

2) Knockdown of Pax6 in human cell line (Hek293 and NA2) using human siRNA, the sequences are below:

Human Pax6 siRNA1:
(forward)
(SEQ ID NO: 33)
5′-GGCAAUCGGUGGUAGUAAATT-3′
(reverse)
(SEQ ID NO: 34)
5′-UUUACUACCACCGAUUGCCCT-3′
Human Pax6 siRNA2:
(forward)
(SEQ ID NO: 35)
5′-CAAGCGUGUCAUCAAUAAATT-3′
(reverse)
(SEQ ID NO: 36)
5′-UUUAUUGAUGACACGCUUGGT-3′

### Results

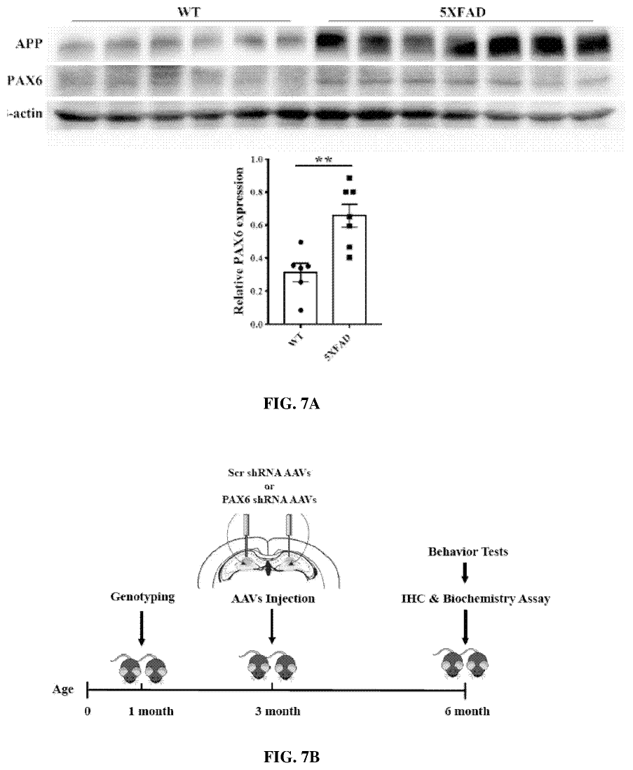

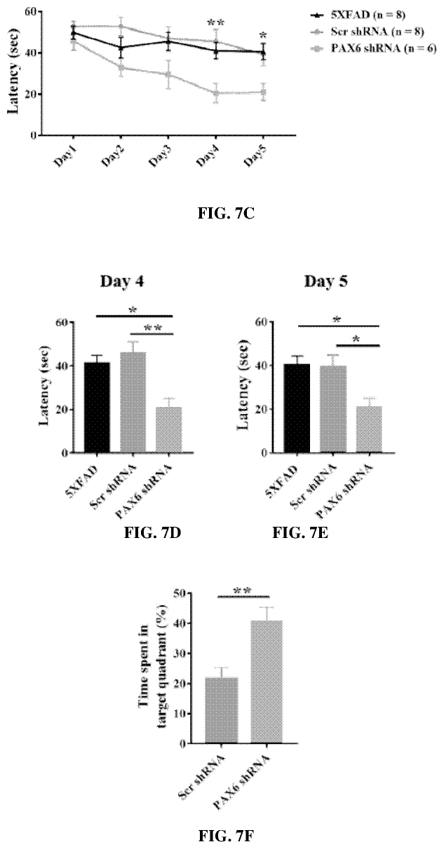

Additional experiments were designed to test the affect of reduced Pax6 expression in in AD mice. Direct knock down of Pax6 expression by stereotaxic injection of Pax6 shRNA AAVs in the hippocampus in AD mice (FIG. 7B) lead to significant improvement in the learning and memory ability of AD mice (FIG. 7C-7F).

Taken together, these Examples showed that Pax6 is an important molecular link between amyloid β and tau hyperphosphorylation. Pax6 expression is increased in the brains of APP transgenic mice and human Alzheimer's disease patients. Importantly, the in vitro and in vivo findings are corroborated by the observation that expression of Pax6 and E2F1 is increased in the entorhinal cortex neurons of mid-stage Alzheimer's disease patients, who have neurofibrillary tangles (GEO Profile Record GDS2795)⁴⁶. Another report noted that Pax6 expression gradually increases in the hippocampi of Alzheimer's disease patients as the disease progresses, with a 1.2-times increase in incipient, 1.3-times increase in moderate and 1.8-times increase in severe Alzheimer's disease cases⁴⁷. c-Myb expression follows the same pattern, with a 3.1-times increase for incipient cases, 2.9-times increase for moderate cases and 5.1-times increase for severe cases (GEO Profile Record GDS810, GDS4136). These findings indicate that Pax6 and c-Myb expression is upregulated throughout the course of Alzheimer's disease.

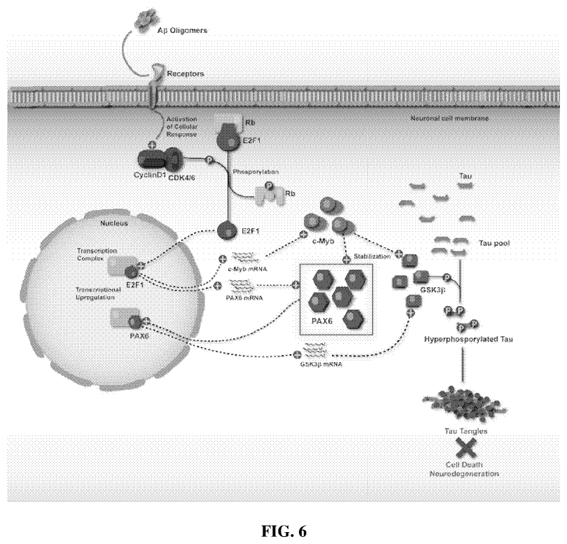

The provided in vitro data supports a mechanism through which amyloid β activates the cellular signaling pathways involving Cdk, pRb and E2F1 by activating downstream transcription factors c-Myb and Pax6, upregulating GSK-33, and ultimately leading to the hyperphosphorylation of tau. In this molecular signaling model (FIG. 6), treatment of primary neurons with amyloid β activates Cdk1 and Cdk4/6 death signals, followed by pRb hyperphosphorylation. Subsequently, transcription factor E2F1 is upregulated, turning on transcription and translation of downstream target genes PAX6 and c-MYB. c-Myb also transactivates Pax6 in response to amyloid β insults. Subsequently, E2F1 and c-Myb synergistically transactivate Pax6. The clinical relevance of the amyloid β-induced upregulation of this pathway is corroborated by microarray data obtained from post-mortem brains of Alzheimer's disease patients and a transgenic APP mouse model. Furthermore, RNAi-mediated downregulation of either PAX6 or c-MYB protects cortical neurons from amyloid β-induced cell death, indicating that both genes have proapoptotic roles. Importantly, we identified a potential target gene for Pax6, GSK-3β, one of the main kinases that phosphorylates tau protein, possibly leading to the formation of neurofibrillary tangles. Results showed that amyloid β neurotoxicity causes Pax6 to transactivate GSK-3β, the upregulation of which was seen in multiple models of Alzheimer's disease, as well as in post-mortem human Alzheimer's disease brains^{48, 49}. Downregulation of Pax6 blocked the amyloid β-induced GSK-3β increase and tau phosphorylation. This interaction indicates a potential signaling pathway linking amyloid β to tau pathology. However, it is known that inhibitory serine-phosphorylation also regulates GSK-3β activity. And in this study, the effect of Pax6 on GSK-3β phosphorylation needs to be further investigated.

Evidence supports the idea that cell cycle re-entry in terminally differentiated neurons causes neuronal death⁵¹. However, how cell cycle signals actually trigger neuronal death is largely unknown. E2F1 has been shown to regulate amyloid β-associated neuronal death dependent on a classic mitochondrial pathway including Bcl-2-associated X protein and caspase-3¹⁹.

The biological activity of tau is modulated by its phosphorylation status. Although previous studies have shown that post-translational modifications by multiple kinases (GSK-3β, Cdk1 and Cdk5, protein kinase A, MAPK) and phosphatases (protein phosphatase 2A and 2B) contribute to tau hyperphosphorylation and neurofibrillary tangles formation^{44, 51, 52}, there is still a missing link in understanding the mechanism of this regulation. GSK-3β, a serine/threonine, proline-directed kinase is involved in a diverse array of signaling pathways, and strongly implicated in Alzheimer's disease pathogenesis. GSK-3β can phosphorylate tau at multiple serine residues, and both its protein level and kinase activity is increased in Alzheimer's disease^{42, 53}. Also, GSK-3β inhibitors have been shown to reduce Alzheimer's disease pathology in vivo and in pre-clinical trials^{54, 55}, Importantly, the provided data showed a new pathway for hyperphosphorylation of tau protein. Although focused on the hyperphosphorylation of tau protein, it was also observed that total tau was reduced after Pax6 knockdown in RNA and protein level (Table 2, below and FIG. 5G).

When first identified as Alzheimer's disease-associated factors, the cause-effect relationship between amyloid β and tau was not clear, but extensive studies have been performed in that regard. For example, Amar et al. shows that amyloid β evoked an increase in tau phosphorylation dependent on CaMKIIa activation⁵⁶. This interaction is supported by the RNA-sequencing data and seems to also involve Pax6 (Table 1, below). Additionally, our RNA-sequencing data (Table 2, below) indicated that Pax6 silencing in neurons could significantly decrease the expression levels of several key kinases that phosphorylate tau, such as Cdk5 (17.3%) and MAPK1 (63.8%). Therefore, there are probably more targets and downstream pathways of Pax6 involved in Alzheimer's disease pathogenesis.

In summary, the experiments above provide evidence that amyloid β neurotoxicity leads to hyperphosphorylation of tau through activation of signaling cascades normally associated with cell-cycle activation in neurons, subsequent activation of transcription factors such as c-Myb and Pax6 and hyperactivation of GSK-3β. The basal endogenous mRNA and protein levels of both c-Myb or Pax6 are almost undetectable in post-mitotic neurons, indicating that Pax6 and c-Myb might be functionally quiescent without stress induction, but are upregulated significantly in Alzheimer's disease brains. Moreover, neurons are dying at all stages of Alzheimer's disease, even if amyloid β is totally blocked. Therefore, a plausible therapeutic strategy is to combine amyloid β removal and targeting Pax6 to prevent neuronal death and tau hyperphosphorylation, therefore to slow Alzheimer's disease progression. The identified pathway indicates E2F1, c-Myb or Pax6 as targets for pharmaceutical intervention.

### Example 10: Palbociclib Reduces Pax6 Expression In Vitro and In Vivo

Experiments were designed to test the effect of palbociclib on Tau in vitro and in vivo. FIGS. 7A-7C show inhibition of Palbociclib on 293T-Tau and 293T-APP cell lines. FIG. 7A shows the results of an XTT assay for choosing the suitable concentrations of Palbociclib. FIG. 7B shows decreased expression levels of CDK4 and Cyclin D1 by adding Palbociclib (10 nM and 50 nM) in 293T-Tau cell line. FIG. 7C shows Decreased expression levels of CDK4 and Cyclin D1 by adding Palbociclib (10 nM and 50 nM) in 293T-APP cell line.

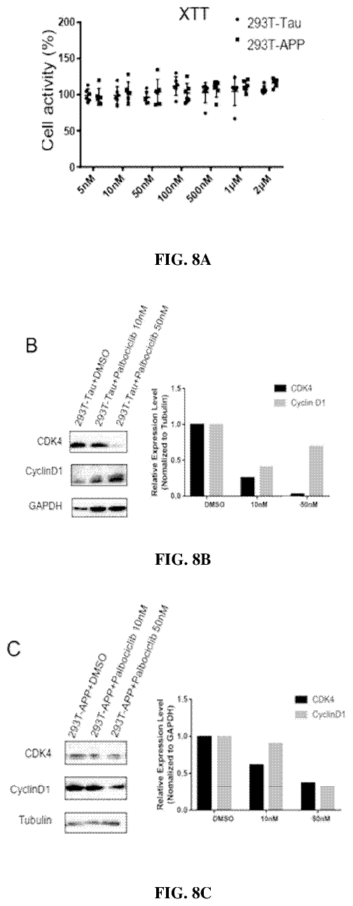

FIGS. 8A-8E show the effects of palbociclib on 5×FAD mice models. FIG. 8A shows increased expression of cell cycle marker (Cyclin D1) and FIG. 8B shows decreased expression of Pax6. FIG. 8C shows a strategy of Palbociclib treatment on 5×FAD mice models. FIG. 8D shows relative decreased expression of E2F-1 in Palbociclib treatment group. FIG. 8E shows relative fewer Aβ deposit in Palbociclib treated mouse brain tissue.

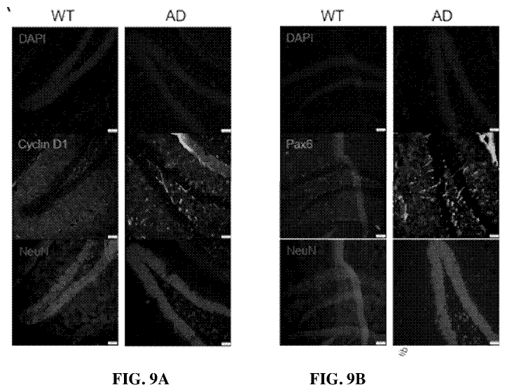

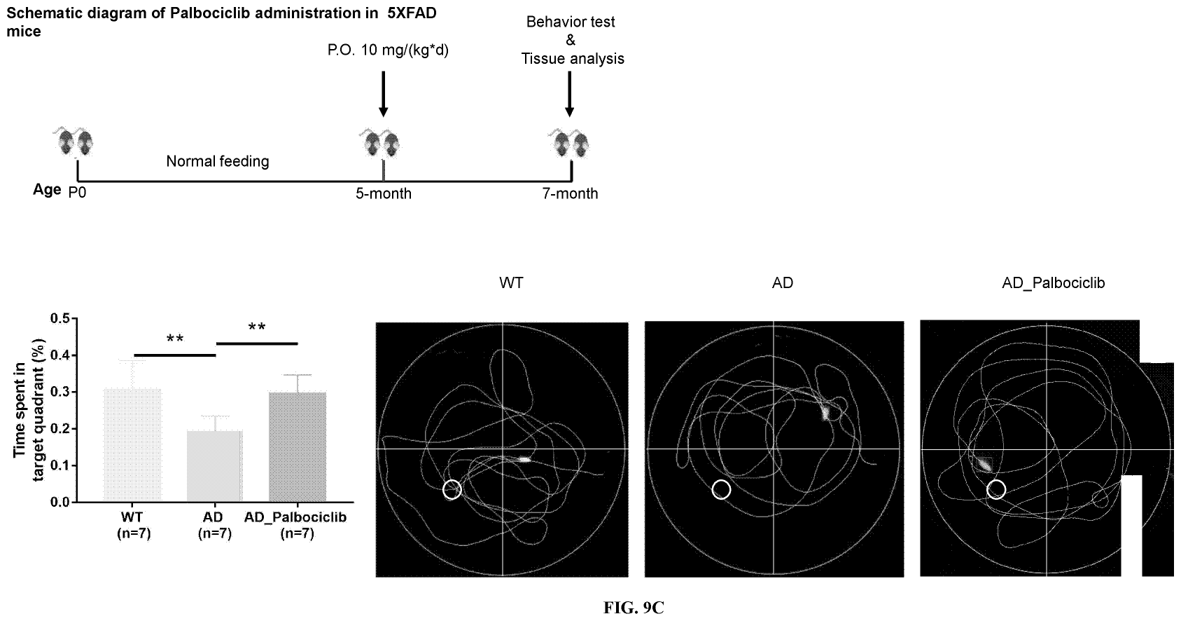

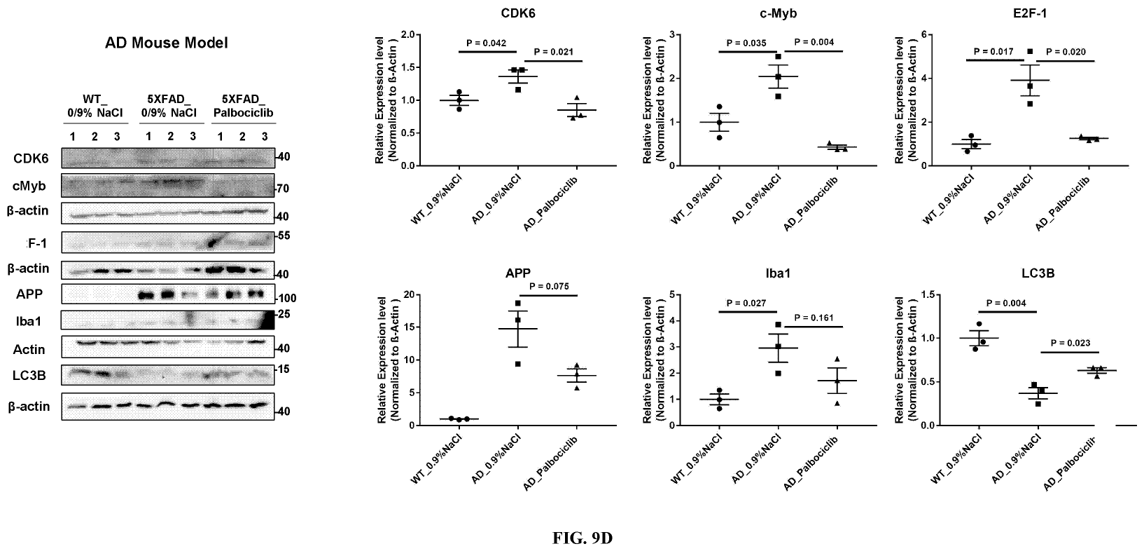

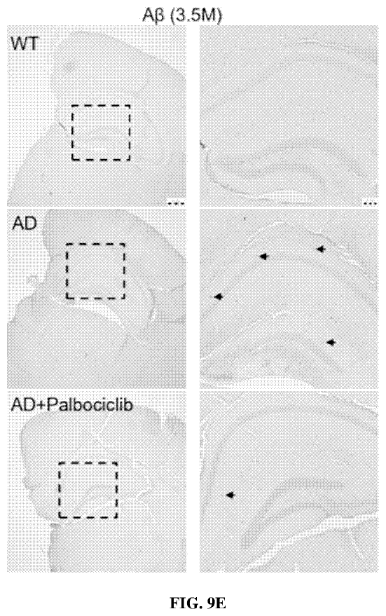

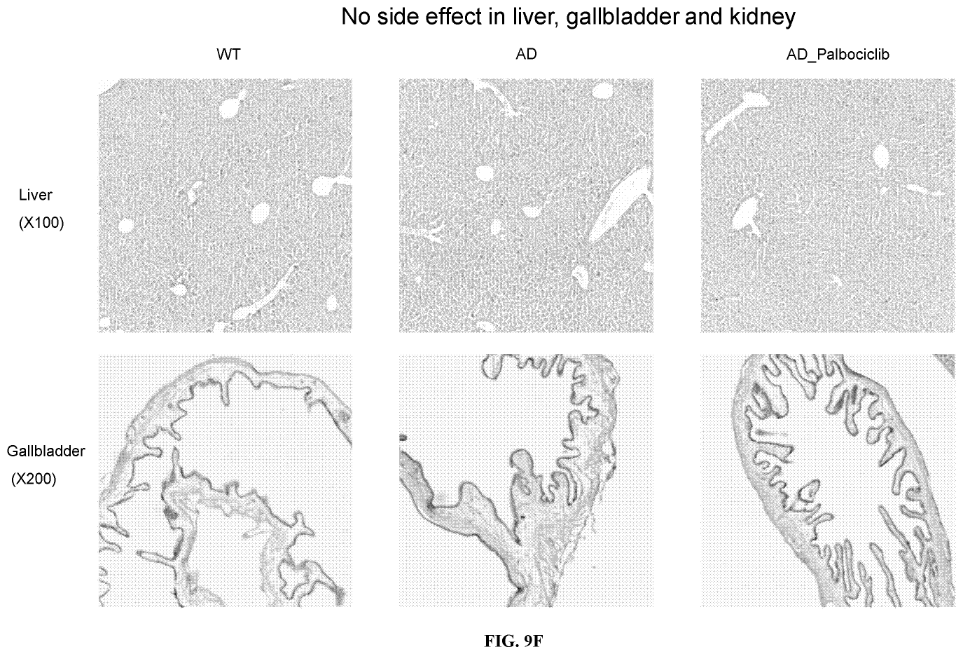

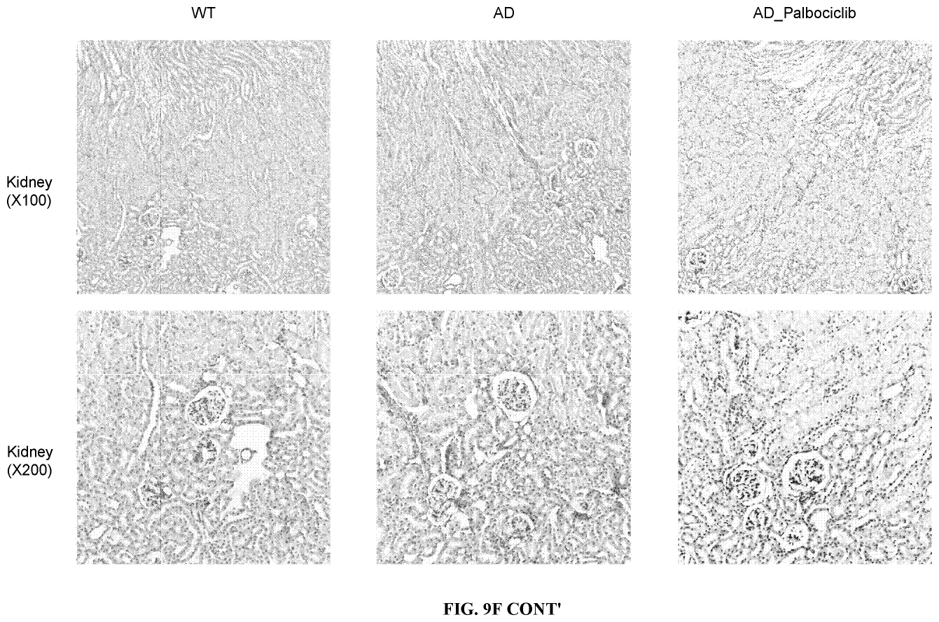

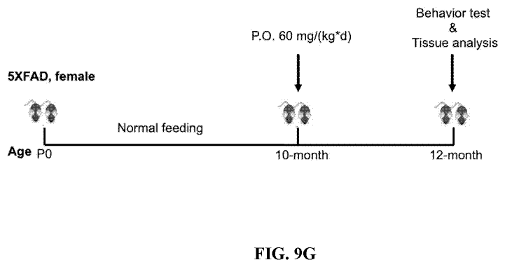

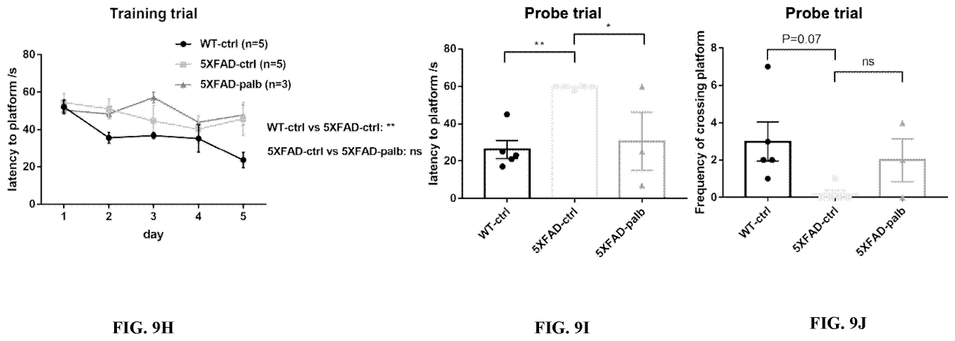

FIGS. 9A-9D show effects of palbociclib on 5×FAD mice model. FIGS. 9A and 9B is a series of immunofluorescent images showing expression of cell cycle marker (Cyclin D1) (9A) and Pax6 (9B). FIG. 9C is a schematic of a strategy for using palbociclib as a treatment on 5×FAD mice models and demonstrated that palbociclib is able to improve memory and learning of 5×FAD mice. FIG. 9D is an image of an autoradiogram of a Western blot and the associated quantitative bar graph showing the relative expression of E2F-1, CDK6, c-Myb, APP, Iba1 and LC3B in palbociclib treatment group compared to controls. FIG. 9E is a series of images of brain tissue showing the relative A3 deposits in palbociclib treated mouse brain tissue compared to controls. FIG. 9F is a series of images of liver, gallbladder and kidney in palbociclib treated mouse and demonstrates that there is no side effect.

FIGS. 9G-9J show the effect of palbociclib treatment on learning and memory ability of 5×FAD female mice in morris water maze. FIG. 9G is a schematic diagram of palbociclib administration with 60 mg/(kg*d); 10-month-old wild-type mice (WT) and 5×FAD mice were administrated with solvent (ctrl) and 60 mg/kg/day palbociclib (palb) for 2 months via oral garage; Morris water maze with n(WT-ctrl)=5, n(5×FAD-ctrl)=5, n(5×FAD-palb)=3 were performed. FIG. 9H shows latency to platform in training trial tested and analyzed by two-way ANOVA test. FIG. 9I shows latency to platform in probe trial of the palbocilib treatment group was significantly improved to the level of the wild type control. FIG. 9J shows frequency of crossing platform in probe trial was significantly increased in palbocilib treatment group.

TABLE 1
List of top 33 down-regulated genes after Pax6 knockdown of murine cortical neurons.

| | | | FPKM | FPKM Pax6 | | | Num of AD- |
|---|---|---|---|---|---|---|---|
| Gene | Description | Chr | Control | knockdown | P-value | Fold_change | pathway |
| AKT3 | v-akt murine thymoma viral | 1 | 67.8037 | 49.7086 | 0 | 0.733123929 | 17 |
| | oncogene homolog 3 | | | | | | |
| AKT1 | v-akt murine thymoma viral | 12 | 96.3654 | 79.0447 | 2.18E−05 | 0.820259896 | 17 |
| | oncogene homolog 1 | | | | | | |
| PRKCB | protein kinase C beta | 7 | 37.908 | 15.439 | 0 | 0.407276365 | 13 |
| GSK3B | glycogen synthase kinase 3 beta | 16 | 64.4706 | 51.0862 | 8.30E−09 | 0.792394625 | 10 |
| MAPK10 | mitogen-activated protein | 5 | 56.5325 | 40.572 | 1.22E−03 | 0.717674716 | 9 |
| | kinase 10 | | | | | | |
| PDGFRA | platelet-derived growth factor | 5 | 23.1794 | 12.8228 | 0 | 0.553199593 | 8 |
| | receptor, alpha polypeptide | | | | | | |
| CALM3 | calmodulin 3 (phosphorylase | 7 | 435.506 | 201.686 | 0 | 0.463107812 | 7 |
| | kinase, delta) | | | | | | |
| CALM1 | calmodulin 1 (phosphorylase | 12 | 528.999 | 281.5 | 0 | 0.532136032 | 7 |
| | kinase, delta) | | | | | | |
| CAMK2A | calcium/calmodulin-dependent | 18 | 27.6371 | 5.168 | 0 | 0.186994793 | 6 |
| | protein kinase II alpha | | | | | | |
| GNAQ | guanine nucleotide binding | 19 | 70.9571 | 42.8375 | 0 | 0.603710584 | 6 |
| | protein, q polypeptide | | | | | | |
| MTOR | mechanistic target of rapamycin | 4 | 13.5587 | 10.7284 | 1.85E−03 | 0.791252465 | 6 |
| MET | met proto-oncogene | 6 | 21.461 | 5.44706 | 0 | 0.253811616 | 5 |
| CACNA1C | calcium channel, voltage- | 6 | 7.39294 | 4.79645 | 2.79E−13 | 0.648787532 | 5 |
| | dependent, L type, alpha 1C | | | | | | |
| | subunit | | | | | | |
| BOL2L1 | BCL2-like 1 | 2 | 38.78 | 25.5494 | 1.15E−11 | 0.658830386 | 5 |
| PAK3 | p21 protein-activated kinase 3 | X | 48.3579 | 35.5706 | 5.17E−06 | 0.735570741 | 5 |
| ELK1 | member of ETS oncogene family | X | 23.3989 | 17.1606 | 3.80E−04 | 0.733395336 | 5 |
| ADCY1 | adenylate cyclase 1 (brain) | 11 | 44.5345 | 16.3566 | 0 | 0.367508074 | 4 |
| ADCY8 | adenylate cyclase 8 (brain) | 15 | 5.17553 | 2.8781 | 1.82E−06 | 0.556097209 | 4 |
| GRIN2D | glutamate receptor, ionotropic | 7 | 2.86404 | 1.27414 | 2.79E−04 | 0.444877488 | 4 |
| | N-methyl D-aspartate 2D | | | | | | |
| GRIN2A | glutamate receptor, ionotropic, | 16 | 1.08763 | 0.816423 | 4.93E−02 | 0.750643603 | 4 |
| | N-methyl D-aspartate 2A | | | | | | |
| FGF10 | fibroblast growth factor 10 | 13 | 1.85963 | 0.303801 | 0 | 0.163366039 | 3 |
| GRIN2B | glutamate receptor, ionotropic, | 6 | 17.1092 | 6.86708 | 0 | 0.401368694 | 3 |
| | N-methyl D-aspartate 2B | | | | | | |
| FGF13 | fibroblast growth factor 13 | X | 64.6106 | 31.8196 | 0 | 0.492484423 | 3 |
| PIP4K2B | phosphatidylinositol-5-phosphate | 11 | 54.9638 | 32.4133 | 0 | 0.589720397 | 3 |
| | 4-kinase, type II, beta | | | | | | |
| ADCY2 | adenylate cyclase 2 (brain) | 13 | 11.0661 | 6.61414 | 0 | 0.597693186 | 3 |
| ADCY3 | adenylate cyclase 3 | 12 | 9.038 | 5.33056 | 8.50E−07 | 0.589794386 | 3 |
| FGF9 | fibroblast growth factor 9 | 14 | 3.65299 | 1.58025 | 1.86E−04 | 0.432592333 | 3 |
| FGF11 | fibroblast growth factor 11 | 11 | 12.5428 | 9.6785 | 2.29E−04 | 0.771640088 | 3 |
| CBLB | Cbl proto-oncogene, E3 | 16 | 8.44287 | 6.73555 | 4.94E−04 | 0.797779524 | 3 |
| | ubiquitin protein ligase 8 | | | | | | |
| FGF12 | fibroblast growth factor 12 | 16 | 47.0354 | 21.7656 | 5.10E−04 | 0.46274643 | 3 |
| FGF18 | fibroblast growth factor 18 | 11 | 3.27264 | 1.43501 | 2.08E−03 | 0.438488223 | 3 |
| ACTN3 | actinin, alpha 3 | 19 | 0.764576 | 0.16726 | 2.56E−02 | 0.218761384 | 3 |
| | (gene/pseudogene) | | | | | | |
| MAPK11 | mitogen-activated protein | 15 | 7.65339 | 6.05901 | 3.95E−02 | 0.791677086 | 3 |
| | kinase 11 | | | | | | |

Gene expression was measured by FPKMV values from Cufflinks. Differential gene expression after Pax6 knockdown was calculated by the ratio of FPKMV values after Pax6 knockdown versus controls without Pax6 knockown. Genes included in this table meet all the following three criteria: over 1.2 fold-change of expression, predicted with Pax6 binding sites and involved in at least 3 AD-related enriched KEGG pathways.

TABLE 2
List of Up- and Down-regulated AD genes in KEGG AD pathways after Pax6 knockdown in murine cortical neurons.

| | | | FPKM | FPKM Pax6 | | | |
|---|---|---|---|---|---|---|---|
| Gene | Description | Chr | Control | knockdown | P-value | Fold change | Ensemble Gene ID |
| RGS14 | regulator of G-protein | chr5 | 0.595046 | 0 | 1.90E−09 | | ENSG00000169220 |
| | signaling 14 | | | | | | |
| Cdk5 | cyclin-dependent kinase 5 | chr5 | 29.8019 | 16.7912 | 0 | −0.827702 | ENSG00000164885 |
| Cdk5r1 | cyclin-dependent kinase 5 | chr11 | 117.767 | 61.8499 | 0 | −0.929097 | ENSG00000176749 |
| | Regulatory subunit 1 (p35) | | | | | | |
| Cdk5r2 | cyclin-dependent kinase 5, | chr1 | 37.8234 | 10.0907 | 0 | −1.90626 | ENSG00000171415 |
| | Regulatory subunit 2 (p39) | | | | | | |
| Gsk-3α | glycogen synthase kinase 3 alpha | chr7 | 68.2157 | 48.3399 | 0 | −0.49689 | ENSG00000105723 |
| Gsk-3β | glycogen synthase kinase 3 beta | chr16 | 64.4706 | 51.0862 | 1.65E−09 | −0.335709 | ENSG00000082701 |
| Mark2 | MAP/microtube affinity | chr19 | 20.8975 | 14.1918 | 7.27E−06 | −0.558166 | ENSG00000072518 |
| | regulating kinase 2 | | | | | | |
| Mark3 | MAP/microtube affinity- | chr12 | 41.6144 | 37.9116 | 0.0114247 | −0.134443 | ENSG00000075413 |
| | regulating kinase 3 | | | | | | |
| Camk2α | calcium/calmodulin-dependent | chr5 | 27.6371 | 5.168 | 0 | −2.41893 | ENSG00000070808 |
| | protein kinase II alpha | | | | | | |
| Mapk1 | mitogen-activated protein | chr16 | 120.014 | 92.7229 | 3.27E−11 | −0.37221 | ENSG00000100030 |
| | kinase 1 | | | | | | |
| Mapk3 | mitogen-activated protein | chr7 | 69.8053 | 56.7963 | 1.35E−05 | −0.297487 | ENSG00000102882 |
| | kinase 3 | | | | | | |
| Mapk7 | mitogen-activated protein | chr17 | 24.8728 | 21.0772 | 0.0201523 | −0.238884 | ENSG00000166484 |
| | kinase 7 | | | | | | |
| Mapk8 | mitogen-activated protein | chr10 | 74.8086 | 44.4007 | 0 | −0.752622 | ENSG00000107643 |
| | kinase 8 | | | | | | |
| Mapk9 | mitogen-activated protein | chr5 | 37.7541 | 24.5412 | 0 | −0.621429 | ENSG00000050748 |
| | kinase 9 | | | | | | |
| Mapk10 | mitogen-activated protein | chr4 | 56.5325 | 40.457 | 0.000453893 | −0.478598 | ENSG00000109339 |
| | kinase 10 | | | | | | |
| Mapk11 | mitogen-activated protein | chr22 | 7.65339 | 6.05901 | 0.0205611 | −0.337018 | ENSG00000185386 |
| | kinase 11 | | | | | | |
| MAPT | microtube associated protein Tau | chr17 | 397.267 | 144.579 | 0 | −1.45824 | ENSG00000186868 |
| BACE1 | beta-site APP cleaving enzyme 1 | chr11 | 28.1597 | 19.2703 | 2.79E−12 | −0.54725 | ENSG00000186868 |
| Dyrk2 | dual-specificity tyrosine-(Y)- | chr12 | 20.3691 | 15.457 | 2.11E−10 | −0.398129 | ENSG00000143479 |
| | phosphorylation regulated | | | | | | |
| | kinase 2 | | | | | | |
| Dyrk3 | dual-specificity tyrosine-(Y)- | chr1 | 8.33782 | 5.47424 | 5.14E−08 | −0.60701 | ENSG00000143479 |
| | phosphorylation regulated | | | | | | |
| | kinase 3 | | | | | | |
| Ttbk2 | tau tubulin kinase 2 | chr15 | 20.1784 | 14.6203 | 2.01E−0.6 | −0.464844 | ENSG00000128881 |
| MTOR | mechanistic target of rapamycin | chr1 | 13.5587 | 10.7284 | 0.000708189 | −0.33779 | ENSG00000198793 |
| Cdkn1a | cyclin-dependent kinase | chr6 | 18.4584 | 121.543 | 0 | 2.71912 | ENSG00000124762 |
| | inhibitor 1A (p21, Cip1) | | | | | | |
| Cdkn1b | cyclin-dependent kinase | chr12 | 51.5436 | 82.839 | 0 | 0.684517 | ENSG00000111276 |
| | inhibitor 1B (p27, Kip1) | | | | | | |
| Cdkn1c | cyclin-dependent kinase | chr11 | 4.91336 | 14.4061 | 0 | 1.55189 | ENSG00000129757 |
| | inhibitor 1C (p57, Kip2) | | | | | | |
| E2f1 | E2F transcription factor 1 | chr20 | 7.04682 | 3.77855 | 3.09E−07 | −0.899141 | ENSG00000101412 |
| Ccna2 | cyclin A2 | chr4 | 19.1857 | 12.7177 | 8.20E−09 | −0.593196 | ENSG00000145386 |
| Cendbp1 | cyclin D-type binding-protein 1 | chr15 | 25.7139 | 17.18 | 1.10E−08 | −0.581813 | ENSG00000166946 |

TABLE 3
Case details for western blots in
post-mortem human brain samples.

| Lane | Case No. | Age | Gender | Diagnosis | Source |
|---|---|---|---|---|---|
| 1 | 1280 | 77 | M | Control | CBTB |
| 2 | 1290 | 69 | F | Control | CBTB |
| 3 | 1361 | 70 | F | Control | CBTB |
| 4 | 1367 | 80 | M | Control | CBTB |
| 5 | 1392 | 77 | M | Control | CBTB |
| 6 | 1456 | 70 | F | Control | CBTB |
| 7 | 1480 | 82 | F | Control | CBTB |
| 8 | 1195 | 80 | M | AD | CBTB |
| 9 | 1202 | 75 | M | AD | CBTB |
| 10 | 1206 | 61 | F | AD | CBTB |
| 11 | 1229 | 81 | M | AD | CBTB |
| 12 | 1240 | 75 | F | AD | CBTB |
| 13 | 1248 | 82 | M | AD | CBTB |
| 14 | 1253 | 78 | F | AD | CBTB |
| 15 | 1029 | 72 | F | Control | CBTB |
| 16 | 1049 | 76 | M | Control | CBTB |
| 17 | 1050 | 74 | M | Control | CBTB |
| 18 | 1051 | 74 | M | Control | CBTB |
| 19 | 1110 | 74 | F | Control | CBTB |
| 20 | 1112 | 89 | M | Control | CBTB |
| 21 | 1244 | 82 | M | Control | CBTB |
| 22 | 1094 | 74 | F | AD | CBTB |
| 23 | 1153 | 79 | M | AD | CBTB |
| 24 | 1156 | 82 | F | AD | CBTB |
| 25 | 1160 | 84 | M | AD | CBTB |
| 26 | 1161 | 74 | M | AD | CBTB |
| 27 | 1174 | 67 | M | AD | CBTB |
| 28 | 1183 | 82 | F | AD | CBTB |

CBTB, Canadian Brain Tissue Bank;
F, Female;
M, Male

TABLE 4
Case details for chromatin immunoprecipitation
in post-mortem human brain samples.

| Lane | Case No. | Age | Gender | Diagnosis | Source |
|---|---|---|---|---|---|
| 1 | T-202 | 74 | M | Control | NYBB |
| 2 | T-220 | 57 | F | Control | NYBB |
| 3 | T-256 | 67 | F | Control | NYBB |
| 4 | T-305 | 74 | F | Control | NYBB |
| 5 | T-335 | 64 | M | Control | NYBB |
| 6 | T-343 | 62 | F | Control | NYBB |
| 7 | T-638 | 78 | M | Control | NYBB |
| 8 | T-779 | 76 | F | Control | NYBB |
| 9 | T-4300 | 92 | M | Control | NYBB |
| 10 | T-4523 | 62 | F | control | NYBB |
| 11 | T-247 | 89+ | F | AD | NYBB |
| 12 | T-263 | 77 | F | AD | NYBB |
| 13 | T-267 | 73 | M | AD | NYBB |
| 14 | T-278 | 86 | M | AD | NYBB |
| 15 | T-279 | 83 | M | AD | NYBB |
| 16 | T-293 | 89 | F | AD | NYBB |
| 17 | T-317 | 89+ | F | AD | NYBB |
| 18 | T-344 | 89 | F | AD | NYBB |
| 19 | T-1070 | 89+ | F | AD | NYBB |
| 20 | T-1370 | 63 | F | AD | NYBB |

NYBB, New York Brain Bank of Columbia University;
F, Female;
M. Male

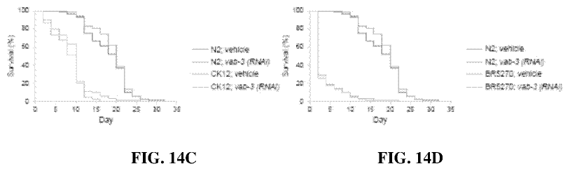

TABLE 5
Results of the Assay of FIGS. 14A-14D

| Group | Mean | SEM | Median |
|---|---|---|---|
| N2 vehicle | 17.98 | 0.55 | 20 |
| N2 vab-3 RNAi | 19.12 | 0.50 | 20 |
| CL2355 vehicle *** | 12.92 | 0.47 | 12 |
| CL2355 vab-3 RNAi | 13.77 | 0.32 | 14 |
| CK12 vehicle *** | 8.10 | 0.40 | 10 |
| CK12 vab-3 RNAi | 9.22 | 0.50 | 10 |
| BR5270 vehicle *** | 3.74 | 0.37 | 2 |
| BR5270 vab-3 RNAi | 3.54 | 0.34 | 2 |

### REFERENCES

1. Canter R Q Penney J, Tsai L H. The road to restoring neural circuits for the treatment of Alzheimer's disease. Nature. Nov. 10 2016; 539(7628):187-196. doi:10.1038/nature20412
2. Edwards F A. A Unifying Hypothesis for Alzheimer's Disease: From Plaques to Neurodegeneration. Trends Neurosci. May 2019; 42(5):310-322. doi:10.1016/j.tins.2019.03.003
3. Bloom G S. Amyloid-beta and tau: the trigger and bullet in Alzheimer disease pathogenesis. JAMA Neurol. April 2014; 71(4):505-8. doi:10.1001/jamaneurol.2013.5847
4. Kant Rvd, Goldstein L S B, Ossenkoppele R. Amyloid-β-independent regulators of tau pathology in Alzheimer disease. Nat Rev Neurosci. January 2020; 21(1):21-35.
5. He Z, Guo J L, McBride J D, et al. Amyloid-beta plaques enhance Alzheimer's brain tau-seeded pathologies by facilitating neuritic plaque tau aggregation. Nat Med. January 2018; 24(1):29-38. doi:10.1038/nm.4443
6. Moh C, Kubiak J Z, Bajic V P, Zhu X, Smith M A, Lee H G Cell cycle deregulation in the neurons of Alzheimer's disease. Results Probl Cell Differ. 2011; 53:565-76. doi:10.1007/978-3-642-19065-0_23
7. Bonda D J, Lee H P, Kudo W, Zhu X, Smith M A, Lee H G Pathological implications of cell cycle re-entry in Alzheimer disease. Expert Rev Mol Med. Jun. 29 2010; 12:e19. doi:10.1017/S146239941000150X
8. Ding X L, Husseman J, Tomashevski A, Nochlin D, Jin L W, Vincent I. The cell cycle Cdc25A tyrosine phosphatase is activated in degenerating postmitotic neurons in Alzheimer's disease. Am J Pathol. December 2000; 157(6):1983-90. doi:S0002-9440(10)64837-7 [pii]10.1016/S0002-9440(10)64837-7
9. Vincent I, Bu B, Hudson K, Husseman J, Nochlin D, Jin L. Constitutive Cdc25B tyrosine phosphatase activity in adult brain neurons with M phase-type alterations in Alzheimer's disease. Neuroscience. 2001; 105(3):639-50. doi:S0306452201002196 [pii]
10. McShea A, Harris P L, Webster K R, Wahl A F, Smith M A. Abnormal expression of the cell cycle regulators P16 and CDK4 in Alzheimer's disease. Am J Pathol. June 1997; 150(6):1933-9.
11. Giovanni A, Wirtz-Brugger F, Keramaris E, Slack R, Park D S. Involvement of cell cycle elements, cyclin-dependent kinases, pRb, and E2F×DP, in B-amyloid-induced neuronal death. J Biol Chem. Jul. 2 1999; 274(27):19011-6.
12. Stone J Q Siedlak S L, Tabaton M, et al. The cell cycle regulator phosphorylated retinoblastoma protein is associated with tau pathology in several tauopathies. J Neuropathol Exp Neurol. July 2011; 70(7):578-87. doi:10.1097/NEN.0b013e3182204414
13. Vincent I, Jicha Q Rosado M, Dickson D W Aberrant expression of mitotic cdc2/cyclin B1 kinase in degenerating neurons of Alzheimer's disease brain. J Neurosci. May 15, 1997; 17(10):3588-98.
14. Smith M Z, Nagy Z, Esiri M M. Cell cycle-related protein expression in vascular dementia and Alzheimer's disease. Neurosci Lett. Aug. 13 1999; 271(1):45-8. doi:S0304394099005091 [pii]
15. Ueberham U, Hessel A, Arendt T. Cyclin C expression is involved in the pathogenesis of Alzheimer's disease. Neurobiol Aging. May-June 2003; 24(3):427-35. doi:S019745800200132X [pii]
16. Arendt T, Rodel L, Gartner U, Holzer M. Expression of the cyclin-dependent kinase inhibitor p16 in Alzheimer's disease. Neuroreport. Nov. 25, 1996; 7(18):3047-9.
17. Arendt T, Holzer M, Gartner U. Neuronal expression of cycline dependent kinase inhibitors of the INK4 family in Alzheimer's disease. J Neural Transm. 1998; 105(8-9):949-60.
18. Luth H J, Holzer M, Gertz H J, Arendt T. Aberrant expression of nNOS in pyramidal neurons in Alzheimer's disease is highly co-localized with p21ras and p16INK4a. Brain Res. Jan. 3, 2000; 852(1):45-55. doi:S0006-8993(99)02178-2 [pii]
19. Giovanni A, Keramaris E, Morris E J, et al. E2F1 mediates death of B-amyloid-treated cortical neurons in a manner independent of p53 and dependent on Bax and caspase 3. J Biol Chem. Apr. 21, 2000; 275(16):11553-60.
20. Chishti M A, Yang D S, Janus C, et al. Early-onset amyloid deposition and cognitive deficits in transgenic mice expressing a double mutant form of amyloid precursor protein 695. J Biol Chem. Jun. 15, 2001; 276(24):21562-70. doi:10.1074/jbc.M100710200 M100710200 [pii]
21. Zhang Y, Parsanejad M, Huang E, et al. Pim-1 kinase as activator of the cell cycle pathway in neuronal death induced by DNA damage. Journal of neurochemistry. January 2010; 112(2):497-510. doi:10.1111/j.1471-4159.2009.06476.x
22. Webster J A, Gibbs J R, Clarke J, et al. Genetic control of human brain transcript expression in Alzheimer disease. Am J Hum Genet. April 2009; 84(4):445-58. doi:10.1016/j.ajhg.2009.03.011
23. Ritchie M E, Phipson B, Wu D, et al. limma powers differential expression analyses for RNA-sequencing and microarray studies. Nucleic acids research. Apr. 20 2015; 43(7):e47. doi:10.1093/nar/gkv007
24. Rosenbloom K R, Armstrong J, Barber G P, et al. The UCSC Genome Browser database: 2015 update. Nucleic acids research. January 2015; 43(Database issue):D670-81. doi:10.1093/nar/gku1177
25. Zhang X, Lan Y, Xu J, et al. CellMarker: a manually curated resource of cell markers in human and mouse. Nucleic acids research. Jan. 8 2019; 47(D1):D721-D728. doi:10.1093/nar/gky900
26. Yu Q Li F, Qin Y, Bo X, Wu Y, Wang S. GOSemSim: an R package for measuring semantic similarity among GO terms and gene products. Bioinformatics. Apr. 1 2010; 26(7):976-8. doi:10.1093/bioinformatics/btq064
27. Trapnell C, Pachter L, Salzberg S L. TopHat: discovering splice junctions with RNA-Seq. Bioinformatics. May 1, 2009; 25(9):1105-11. doi:10.1093/bioinformatics/btp120
28. Trapnell C, Williams B A, Pertea Q et al. Transcript assembly and quantification by RNA-Seq reveals unannotated transcripts and isoform switching during cell differentiation. Nature biotechnology. May 2010; 28(5):511-5. doi:10.1038/nbt.1621
29. Flicek P, Amode M R, Barrell D, et al. Ensembl 2012. Nucleic acids research. January 2012; 40(Database issue):D84-90. doi:10.1093/nar/gkr991
30. Kanehisa M. The KEGG database. Novartis Foundation symposium. 2002; 247:91-101; discussion 101-3, 119-28, 244-52.
31. Zhang Y, Qu D, Morris E J, et al. The Chkl/Cdc25A pathway as activators of the cell cycle in neuronal death induced by camptothecin. J Neurosci. Aug. 23 2006; 26(34):8819-28. doi:26/34/8819 [pii]10.1523/JNEUROSCI.2593-06.2006
32. Barghorn S, Nimmrich V, Striebinger A, et al. Globular amyloid beta-peptide oligomer—a homogenous and stable neuropathological protein in Alzheimer's disease. Journal of neurochemistry. November 2005; 95(3):834-47. doi:10.1111/j.1471-4159.2005.03407.x
33. O'Hare M J, Hou S T, Morris E J, et al. Induction and modulation of cerebellar granule neuron death by E2F-1. J Biol Chem. Aug. 18 2000; 275(33):25358-64. doi:10.1074/jbc.M001725200 M001725200 [pii]
34. Osumi N, Shinohara H, Numayama-Tsuruta K, Maekawa M. Concise review: Pax6 transcription factor contributes to both embryonic and adult neurogenesis as a multifunctional regulator. Stem Cells. July 2008; 26(7):1663-72. doi:2007-0884 [pii]10.1634/stemcells.2007-0884
35. Nomura T, Haba H, Osumi N. Role of a transcription factor Pax6 in the developing vertebrate olfactory system. Dev Growth Differ. December 2007; 49(9):683-90. doi:DGD965 [pii]10.1111/j.1440-169X.2007.00965.x
36. Shaham O, Menuchin Y, Farhy C, Ashery-Padan R. Pax6: a multi-level regulator of ocular development. Prog Retin Eye Res. September 2012; 31(5):351-76. doi:10.1016/j.preteyeres.2012.04.002
37. Khan U A, Liu L, Provenzano F A, et al. Molecular drivers and cortical spread of lateral entorhinal cortex dysfunction in preclinical Alzheimer's disease. Nature neuroscience. February 2014; 17(2):304-11. doi:10.1038/nn.3606
38. Di Paola M, Macaluso E, Carlesimo G A, et al. Episodic memory impairment in patients with Alzheimer's disease is correlated with entorhinal cortex atrophy. A voxel-based morphometry study. Journal of neurology. June 2007; 254(6):774-81. doi:10.1007/s00415-006-0435-1
39. Chauhan B K, Yang Y, Cveklova K, Cvekl A. Functional properties of natural human PAX6 and PAX6(5a) mutants. Invest Ophthalmol Vis Sci. February 2004; 45(2):385-92.
40. Zheng J B, Zhou Y H, Maity T, Liao W S, Saunders G F. Activation of the human PAX6 gene through the exon 1 enhancer by transcription factors SEF and Sp1. Nucleic acids research. Oct. 1 2001; 29(19):4070-8.
41. Lee S E, Simons S B, Heldt S A, et al. RGS14 is a natural suppressor of both synaptic plasticity in CA2 neurons and hippocampal-based learning and memory. Proc Natl Acad Sci USA. Sep. 28 2010; 107(39):16994-8. doi:10.1073/pnas.1005362107
42. Takashima A. GSK-3 is essential in the pathogenesis of Alzheimer's disease. J Alzheimers Dis. 2006; 9(3 Suppl):309-17.
43. Iqbal K, Liu F, Gong C X, Grundke-Iqbal I. Tau in Alzheimer disease and related tauopathies. Current Alzheimer research. December 2010; 7(8):656-64.
44. Mi K, Johnson G V The role of tau phosphorylation in the pathogenesis of Alzheimer's disease. Current Alzheimer research. December 2006; 3(5):449-63.
45. Hoshi M, Sato M, Matsumoto S, et al. Spherical aggregates of beta-amyloid (amylospheroid) show high neurotoxicity and activate tau protein kinase I/glycogen synthase kinase-3beta. Proc Natl Acad Sci USA. May 27 2003; 100(11):6370-5. doi:10.1073/pnas.1237107100 1237107100 [pii]
46. Dunckley T, Beach T Q Ramsey K E, et al. Gene expression correlates of neurofibrillary tangles in Alzheimer's disease. Neurobiol Aging. October 2006; 27(10):1359-71. doi:S0197-4580(05)00222-8 [pii]10.1016/j.neurobiolaging.2005.08.013
47. Blalock E M, Geddes J W, Chen K C, Porter N M, Markesbery W R, Landfield P W Incipient Alzheimer's disease: microarray correlation analyses reveal major transcriptional and tumor suppressor responses. Proc Natl Acad Sci USA. Feb. 17 2004; 101(7):2173-8. doi:10.1073/pnas.0308512100 0308512100 [pii]
48. Hernandez F, Lucas J J, Avila J. GSK3 and tau: two convergence points in Alzheimer's disease. J Alzheimers Dis. 2013; 33 Suppl 1:S141-4. doi:10.3233/JAD-2012-129025
49. Schaffer B A, Bertram L, Miller B L, et al. Association of GSK3B with Alzheimer disease and frontotemporal dementia. Arch Neurol. October 2008; 65(10):1368-74. doi:65/10/1368 [pii]10.1001/archneur.65.10.1368
50. Herrup K. The contributions of unscheduled neuronal cell cycle events to the death of neurons in Alzheimer's disease. Front Biosci (Elite Ed). Jan. 1 2012; 4:2101-9. doi:10.2741/527
51. Martin L, Latypova X, Terro F. Post-translational modifications of tau protein: implications for Alzheimer's disease. Neurochem Int. March 2011; 58(4):458-71. doi:10.1016/j.neuint.2010.12.023
52. Dolan P J, Johnson G V The role of tau kinases in Alzheimer's disease. Curr Opin Drug Discov Devel. September 2010; 13(5):595-603.
53. Leroy K, Yilmaz Z, Brion J P. Increased level of active GSK-3beta in Alzheimer's disease and accumulation in argyrophilic grains and in neurones at different stages of neurofibrillary degeneration. Neuropathol Appl Neurobiol. February 2007; 33(1):43-55. doi:NAN795 [pii]10.1111/j.1365-2990.2006.00795.x
54. Ma T. GSK3 in Alzheimer's disease: mind the isoforms. J Alzheimers Dis. 2014; 39(4):707-10. doi:10.3233/JAD-131661
55. Medina M, Avila J. Glycogen synthase kinase-3 (GSK-3) inhibitors for the treatment of Alzheimer's disease. Curr Pharm Des. 2010; 16(25):2790-8. doi:10.2174/138161210793176581
56. Amar F, Sherman M A, Rush T, et al. The amyloid-beta oligomer Abeta*56 induces specific alterations in neuronal signaling that lead to tau phosphorylation and aggregation. Sci Signal. May 9 2017; 10(478)doi:10.1126/scisignal.aal2021
   Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of reducing Tau phosphorylation or total Tau in neurons of a subject in need thereof comprising administering the subject an effective amount of a direct or indirect inhibitor of Pax6 (Pax6 inhibitor).

2. The method of claim 1, wherein the subject has a proteinopathy, amyloidosis, or a tauopathy.

3. A method of treating a proteinopathy, amyloidosis, or a tauopathy comprising administering the subject an effective amount of Pax6 inhibitor.

4. (canceled)

5. The method of claim 1, wherein the tauopathy is selected from Alzheimer's disease, Frontotemporal lobar degeneration (FTLD), autism, epilepsy, depression, stroke, Dravet syndrome or a seizure disorder.

6. The method of claim 1, wherein the Pax6 inhibitor is effective to reduce the formation amyloid β plaques, reduce the formation of neurofibrillary tangles, or a combination thereof in the subject.

7. The method of claim 1, where the Pax6 inhibitor is effective to reduce neuronal cell death in the subject.

8. The method of claim 1, wherein the Pax6 inhibitor is a small molecule or a functional nucleic acid.

9. The method of claim 1, wherein the Pax6 inhibitor is the small molecule palbociclib, flavopiridol, abemaciclib, ribociclib, apigenin, ICCB280, diclofenac, indomethacin, non-steroidal anti-inflammatory (NSAIDs) drugs, (−)-kusunokinin, bortezomib (BZB), valproic acid (VPA), bigelovin, eugenol, emodin, icilin, NSC69603, gambogic acid, tolfenamic acid, HDAC inhibitors such as oxamflatin, 4-Allyl-2-methoxyphenol (eugenol), piperlongumine, Delta 9-tetrahydrocannabinol, bortezomib, sorafenib, dracorhodin perchlorate, triptolide fangchinoline, PD-0332991, methyl gallate, or a derivative, stereoisomer, or pharmaceutically acceptable salt thereof.

10. The method of claim 1, wherein the Pax6 inhibitor is a functional nucleic acid selected from the group consisting of antisense molecules, siRNA, shRNA, miRNA, G-quadruplex, aptamers, ribozymes, triplex forming molecules, RNAi, and external guide sequences that targets the Pax6 gene or a gene product thereof.

11. The method of claim 1, wherein the Pax6 inhibitor is an siRNA, shRNA, or miRNA, or a nucleic acid expression construct encoding an siRNA, shRNA, or miRNA, wherein the siRNA, shRNA, or miRNA targets any one of SEQ ID NOS: 1-7, or a nucleic acid encoding the polypeptide of any one of SEQ ID NOS: 8-14, or a variant of any of the foregoing sequences with at least 65% sequence identity thereto, optionally wherein the nucleic acid expression construct is a plasmid or a virus or viral vector, optionally wherein the virus or viral vector is adeno-associated viruses (AAV).

12. The method of claim 11, wherein the miRNA is miR-670 and miR-692, miR215.

13. The method of claim 1, wherein the Pax6 inhibitor is a small activating RNA (saRNA).

14. The method of claim 13, wherein the saRNA is CEBPA-saRNA.

15. The method of claim 1 wherein the Pax6 inhibitor is targeted to the brain.

16. The method of claim 1 wherein the Pax6 inhibitor is targeted to neurons.

17. The method of claim 1, wherein the Pax6 inhibitor is administered to the subject by an oral, parenteral, transdermal, or transmucosal administration, optionally wherein the transmucosal administration is intranasal.

18. The method of claim 1, wherein the Pax6 inhibitor is administered to the subject locally or systemically.

19. The method of claim 1, wherein the inhibitor is packaged in a delivery vehicle, optionally wherein the delivery vehicle is liposomes.

20. A pharmaceutical composition comprising an effective amount of a Pax6 inhibitor to reducing Tau phosphorylation in neurons in a subject in need thereof.


SEQUENCE LISTING (36 sequences)

SEQ ID NO: 1 - DNA, 2500 bp, Homo sapiens
agcataggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaa

SEQ ID NO: 2 - DNA, 6922 bp, Homo sapiens
accctcttttcttatcattgacatttaaactctggggcaggtcctcgcgtagaacgcggctgtcagatctgccacttcccctgccgagcggcggtgagaagtgtgggaaccggcgctgccaggctcacctgcctccccgccctccgctcccaggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcagacccatgcagatgcaaaagtccaagtgctggacaatcaaaacgtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaacttaagtgttttgaagttgttcacaactacatatcaaaattaaccattgttgattgtaaaaaaccatgccaaagcctttgtatttcctttattatacagttttctttttaaccttatagtgtggtgttacaaattttatttccatgttagatcaacattctaaaccaatggttactttcacacacactctgttttacatcctgatgatccttaaaaaataatccttatagataccataaatcaaaaacgtgttagaaaaaaattccacttacagcagggtgtagatctgtgcccatttatacccacaacatatatacaaaatggtaacatttcccagttagccatttaattctaaagctcaaagtctagaaataatttaaaaatgcaacaagcgattagctaggaattgttttttgaattaggactggcattttcaatctgggcagatttccattgtcagcctatttcaacaatgatttcactgaagtatattcaaaagtagatttcttaaaggagactttctgaaagctgttgcctttttcaaataggccctctcccttttctgtctccctcccctttgcacaagaggcatcatttcccattgaaccactacagctgttcccatttgaatcttgctttctgtgcggttgtggatggttggagggtggaggggggatgttgcatgtcaaggaataatgagcacagacacatcaacagacaacaacaaagcagactgtgactggccggtgggaattaaaggccttcagtcattggcagcttaagccaaacattcccaaatctatgaagcagggcccattgttggtcagttgttatttgcaatgaagcacagttctgatcatgtttaaagtggaggcacgcagggcaggagtgcttgagcccaagcaaaggatggaaaaaaataagcctttgttgggtaaaaaaggactgtctgagactttcatttgttctgtgcaacatataagtcaatacagataagtcttcctctgcaaacttcactaaaaagcctgggggttctggcagtctagattaaaatgcttgcacatgcagaaacctctggggacaaagacacacttccactgaattatactctgctttaaaaaaatccccaaaagcaaatgatcagaaatgtagaaattaatggaaggatttaaacatgaccttctcgttcaatatctactgttttttagttaaggaattacttgtgaacagataattgagattcattgctccggcatgaaatatactaataattttattccaccagagttgctgcacatttggagacaccttcctaagttgcagtttttgtatgtgtgcatgtagttttgttcagtgtcagcctgcactgcacagcagcacatttctgcaggggagtgagcacacatacgcactgttggtacaattgccggtgcagacatttctacctcctgacattttgcagcctacattccctgagggctgtgtgctgagggaactgtcagagaagggctatgtgggagtgcatgccacagctgctggctggcttacttcttccttctcgctggctgtaatttccaccacggtcaggcagccagttccggcccacggttctgttgtgtagacagcagagactttggagacccggatgtcgcacgccaggtgcaagaggtgggaatgggagaaaaggagtgacgtgggagcggagggtctgtatgtgtgcacttgggcacgtatatgtgtgctctgaaggtcaggattgccagggcaaagtagcacagtctggtatagtctgaagaagcggctgctcagctgcagaagccctctggtccggcaggatgggaacggctgccttgccttctgcccacaccctagggacatgagctgtccttccaaacagagctccaggcactctcttggggacagcatggcaggctctgtgtggtagcagtgcctgggagttggccttttactcattgttgaaataatttttgtttattatttatttaacgatacatatatttatatatttatcaatggggtatctgcagggatgttttgacaccatcttccaggatggagattatttgtgaagacttcagtagaatcccaggactaaacgtctaaattttttctccaaacttgactgacttgggaaaaccaggtgaatagaataagagctgaatgttttaagtaataaacgttcaaactgctctaagtaaaaaaatgcattttactgcaatgaatttctagaatatttttcccccaaagctatgcctcctaacccttaaatggtgaacaactggtttcttgctacagctcactgccatttcttcttactatcatcactaggtttcctaagattcactcatacagtattatttgaagattcagctttgttctgtgaatgtcatcttaggattgtgtctatattcttttgcttatttctttttactctgggcctctcatactagtaagattttaaaaagccttttcttctctgtatgtttggctcaccaaggcgaaatatatattcttctctttttcatttctcaagaataaacctcatctgcttttttgtttttctgtgttttggcttggtactgaatgactcaactgctcggttttaaagttcaaagtgtaagtacttagggttagtactgcttatttcaataatgttgacggtgactatctttggaaagcagtaacatgctgtcttagaaatgacattaataatgggcttaaacaaatgaataggggggtccccccactctccttttgtatgcctatgtgtgtctgatttgttaaaagatggacagggaattgattgcagagtgtcgcttccttctaaagtagttttattttgtctactgttagtatttaaagatcctggaggtggacataaggaataaatggaagagaaaagtagatattgtatggtggctactaaaaggaaattcaaaaagtcttagaacccgagcacctgagcaaactgcagtagtcaaaatatttatctcatgttaaagaaaggcaaatctagtgtaagaaatgagtaccatatagggttttgaagttcatatactagaaacacttaaaagatatcatttcagatattacgtttggcattgttcttaagtatttatatctttgagtcaagctgataattaaaaaaaatctgttaatggagtgtatatttcataatgtatcaaaatggtgtctatacctaaggtagcattattgaagagagatatgtttatgtagtaagttattaacataatgagtaacaaataatgtttccagaagaaaggaaaacacattttcagagtgcgtttttatcagaggaagacaaaaatacacacccctctccagtagcttatttttacaaagccggcccagtgaattagaaaaacaaagcacttggatatgatttttggaaagcccaggtacacttattattcaaaatgcacttttactgagtttgaaaagtttcttttatatttaaaataagggttcaaatatgcatattcaatttttatagtagttatctatttgcaaagcatatattaactagtaattggctgttaattttatagacatggtagccagggaagtatatcaatgacctattaagtattttgacaagcaatttacatatctgatgacctcgtatctctttttcagcaagtcaaatgctatgtaattgttccattgtgtgttgtataaaatgaatcaacacggtaagaaaaaggttagagttattaaaataataaactgactaaaatactcatttgaatttattcagaatgttcataatgctttcaaaggacatagcagagcttttgtggagtatccgcacaacattatttattatctatggactaaatcaattttttgaagttgctttaaaatttaaaagcacctttgcttaatataaagccctttaattttaactgacagatcaattctgaaactttattttgaaaagaaaatggggaagaatctgtgtctttagaattaaaagaaatgaaaaaaataaacccgacattctaaaaaaatagaataagaaacctgatttttagtactaatgaaatagcgggtgacaaaatagttgtctttttgattttgatcacaaaaaataaactggtagtgacaggatatgatggagagatttgacatcctggcaaatcactgtcattgattcaattattctaattctgaataaaagctgtatacagta

SEQ ID NO: 3 - DNA, 6863 bp, Homo sapiens
gcacttagtcaacaaatggcacgtgggagaagttggtgagtgtttggtgaggactcttcagggcttttcacaagaaccctctgtacacaaagaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcagacccatgcagatgcaaaagtccaagtgctggacaatcaaaacgtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaacttaagtgttttgaagttgttcacaactacatatcaaaattaaccattgttgattgtaaaaaaccatgccaaagcctttgtatttcctttattatacagttttctttttaaccttatagtgtggtgttacaaattttatttccatgttagatcaacattctaaaccaatggttactttcacacacactctgttttacatcctgatgatccttaaaaaataatccttatagataccataaatcaaaaacgtgttagaaaaaaattccacttacagcagggtgtagatctgtgcccatttatacccacaacatatatacaaaatggtaacatttcccagttagccatttaattctaaagctcaaagtctagaaataatttaaaaatgcaacaagcgattagctaggaattgttttttgaattaggactggcattttcaatctgggcagatttccattgtcagcctatttcaacaatgatttcactgaagtatattcaaaagtagatttcttaaaggagactttctgaaagctgttgcctttttcaaataggccctctcccttttctgtctccctcccctttgcacaagaggcatcatttcccattgaaccactacagctgttcccatttgaatcttgctttctgtgcggttgtggatggttggagggtggaggggggatgttgcatgtcaaggaataatgagcacagacacatcaacagacaacaacaaagcagactgtgactggccggtgggaattaaaggccttcagtcattggcagcttaagccaaacattcccaaatctatgaagcagggcccattgttggtcagttgttatttgcaatgaagcacagttctgatcatgtttaaagtggaggcacgcagggcaggagtgcttgagcccaagcaaaggatggaaaaaaataagcctttgttgggtaaaaaaggactgtctgagactttcatttgttctgtgcaacatataagtcaatacagataagtcttcctctgcaaacttcactaaaaagcctgggggttctggcagtctagattaaaatgcttgcacatgcagaaacctctggggacaaagacacacttccactgaattatactctgctttaaaaaaatccccaaaagcaaatgatcagaaatgtagaaattaatggaaggatttaaacatgaccttctcgttcaatatctactgttttttagttaaggaattacttgtgaacagataattgagattcattgctccggcatgaaatatactaataattttattccaccagagttgctgcacatttggagacaccttcctaagttgcagtttttgtatgtgtgcatgtagttttgttcagtgtcagcctgcactgcacagcagcacatttctgcaggggagtgagcacacatacgcactgttggtacaattgccggtgcagacatttctacctcctgacattttgcagcctacattccctgagggctgtgtgctgagggaactgtcagagaagggctatgtgggagtgcatgccacagctgctggctggcttacttcttccttctcgctggctgtaatttccaccacggtcaggcagccagttccggcccacggttctgttgtgtagacagcagagactttggagacccggatgtcgcacgccaggtgcaagaggtgggaatgggagaaaaggagtgacgtgggagcggagggtctgtatgtgtgcacttgggcacgtatatgtgtgctctgaaggtcaggattgccagggcaaagtagcacagtctggtatagtctgaagaagcggctgctcagctgcagaagccctctggtccggcaggatgggaacggctgccttgccttctgcccacaccctagggacatgagctgtccttccaaacagagctccaggcactctcttggggacagcatggcaggctctgtgtggtagcagtgcctgggagttggccttttactcattgttgaaataatttttgtttattatttatttaacgatacatatatttatatatttatcaatggggtatctgcagggatgttttgacaccatcttccaggatggagattatttgtgaagacttcagtagaatcccaggactaaacgtctaaattttttctccaaacttgactgacttgggaaaaccaggtgaatagaataagagctgaatgttttaagtaataaacgttcaaactgctctaagtaaaaaaatgcattttactgcaatgaatttctagaatatttttcccccaaagctatgcctcctaacccttaaatggtgaacaactggtttcttgctacagctcactgccatttcttcttactatcatcactaggtttcctaagattcactcatacagtattatttgaagattcagctttgttctgtgaatgtcatcttaggattgtgtctatattcttttgcttatttctttttactctgggcctctcatactagtaagattttaaaaagccttttcttctctgtatgtttggctcaccaaggcgaaatatatattcttctctttttcatttctcaagaataaacctcatctgcttttttgtttttctgtgttttggcttggtactgaatgactcaactgctcggttttaaagttcaaagtgtaagtacttagggttagtactgcttatttcaataatgttgacggtgactatctttggaaagcagtaacatgctgtcttagaaatgacattaataatgggcttaaacaaatgaataggggggtccccccactctccttttgtatgcctatgtgtgtctgatttgttaaaagatggacagggaattgattgcagagtgtcgcttccttctaaagtagttttattttgtctactgttagtatttaaagatcctggaggtggacataaggaataaatggaagagaaaagtagatattgtatggtggctactaaaaggaaattcaaaaagtcttagaacccgagcacctgagcaaactgcagtagtcaaaatatttatctcatgttaaagaaaggcaaatctagtgtaagaaatgagtaccatatagggttttgaagttcatatactagaaacacttaaaagatatcatttcagatattacgtttggcattgttcttaagtatttatatctttgagtcaagctgataattaaaaaaaatctgttaatggagtgtatatttcataatgtatcaaaatggtgtctatacctaaggtagcattattgaagagagatatgtttatgtagtaagttattaacataatgagtaacaaataatgtttccagaagaaaggaaaacacattttcagagtgcgtttttatcagaggaagacaaaaatacacacccctctccagtagcttatttttacaaagccggcccagtgaattagaaaaacaaagcacttggatatgatttttggaaagcccaggtacacttattattcaaaatgcacttttactgagtttgaaaagtttcttttatatttaaaataagggttcaaatatgcatattcaatttttatagtagttatctatttgcaaagcatatattaactagtaattggctgttaattttatagacatggtagccagggaagtatatcaatgacctattaagtattttgacaagcaatttacatatctgatgacctcgtatctctttttcagcaagtcaaatgctatgtaattgttccattgtgtgttgtataaaatgaatcaacacggtaagaaaaaggttagagttattaaaataataaactgactaaaatactcatttgaatttattcagaatgttcataatgctttcaaaggacatagcagagcttttgtggagtatccgcacaacattatttattatctatggactaaatcaattttttgaagttgctttaaaatttaaaagcacctttgcttaatataaagccctttaattttaactgacagatcaattctgaaactttattttgaaaagaaaatggggaagaatctgtgtctttagaattaaaagaaatgaaaaaaataaacccgacattctaaaaaaatagaataagaaacctgatttttagtactaatgaaatagcgggtgacaaaatagttgtctttttgattttgatcacaaaaaataaactggtagtgacaggatatgatggagagatttgacatcctggcaaatcactgtcattgattcaattattctaattctgaataaaagctgtatacagtaaaa

SEQ ID NO: 4 - DNA, 2603 bp, Homo sapiens
gcatgttgcggagtgattagtgggtttgaaaagggaaccgtggctcggcctcatttcccgctctggttcaggcgcaggaggaagtgttttgctggaggatgatgacagaggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaaaaaaaaaaaggaaaggaaatattgtgttaattcagtcagtgactatggggacacaacagttgagctttcaggaaagaaagaaaaatggctgttagagccgcttcagttctacaattgtgtcctgtattgtaccactggggaaggaatggacttgaaacaaggacctttgtatacagaaggcacgatatcagttggaacaaatcttcattttggtatccaaacttttattcattttggtgtattatttgtaaatgggcatttgtatgttataatgaaaaaaagaacaatgtagactggatggatgtttgatctgtgttggtcatgaagttgttttttttttttttaaaaagaaaaccatgatcaacaagctttgccacgaatttaagagttttatcaagatatatcgaatacttctacccatctgttcatagtttatggactgatgttccaagtttgtatcattcctttgcatataattaaacctggaacaacatgcactagatttatgtcagaaatatctgttggttttccaaaggttgttaacagatgaagtttatgtgcaaaaaagggtaagatataaattcaaggaagaaaaaaagttgatagctaaaaggtagagtgtgtcttcgatataatccaatttgttttatgtcaaaatgtaagtatttgtcttccctagaaatcctcagaatgatttctataataaagttaatttcatttatatttgacaagaatatagatgttttatacacattttcatgcaatcatacgtttcttttttggccagcaaaagttaattgttcttagatatagttgtattactgttcacggtccaatcattttgtgcatctagagttcattcctaatcaattaaaagtgcttgcaagagttttaaa

SEQ ID NO: 5 - DNA, 12064 bp, Drosophila melanogaster
atcgatttcgggcagtcgataaaacaacagttcggagtgcagcaaaagtgcgggaaatctgcggaaatgttattaatcgaacattaagggatacctcgcccagccgcgctctagcattcaccatttccctttgtttgcggcggcggcgctcgttgtacggccgtgcgaatcgagagaaaaacgcataaaatcggggaaagtgcactagccccgtgctgcaatgtaaataaacaaggctctgatgagcagttgccgatcaagtgcttcgattagccagcaaaatgtgcgtacaaggcgagaatcagtgacgaaaacaacaactacgtgctgctgcgttcattgacgtgacgcgcgagagtgaatgtgtgcgtgcgtgtgtgtttgtgtgtgtgtgctggctgctggcgagtgagtgtgtgcgtgctccccatatgtgtgttgtcgaaaaagcgtttttcaaaccacaagccagcaacaaacaaatcacaacagcacgtcacaaattgcaatagaagcagaagcagcaacacctcaaatcaaaaaaaggccccacaacaacaacatctgtcgagttctcagagagtgagaaaaagaaagagcgcgttagagtgagagaggggcaaaaagaaagtcaacagtcgcgtgtgaaatgaaattcgaaagatgtccgcgtgtgtgtgtgttgagtgtatagaattgagtaagaagcagcgcggcaaaaggaaataagcaaaagcaatagccaaaaggaaccaacaaccagcagcaacaaattacccaatgaatcaatcaaaattggcataatttgcattcgccgacgttattgtctataaaacccccctgaaaaaaacaccacacaacagaagagcagcacaaatcttttgccaatgagtcgcagacagtcattggatcgaccaggaatattgtctcctccgggtccgtttctgacgcccagtccatcgccatcgatctcagctagccctcgtctgcaaccatcgccatcgctctcgccgttcccacaggatgtggtgctcgtagccggtggcagccaagtaaacgccaacagcaatccccaggaacacgagcgcaaggcggcgacgcctggcaggagacagtccatccaccagttcaccaatggcagtcccaatgctggaactgtcgtcttccagccgagcccctcacaatcgccggccgctgccacgacggtcatcggcgtgaccagtggtggcctcatagccaccgccgccggaggaactggagccggtctgagtaccggagtaggagccgggagcagttccggagtaggaattggcgtcgcgctgcacggaaatgccatcggcaacgagctgaatgccactctggtgggatcgaataatatccagctcaataagaagggcacaaagcgggcaacccagcagcagcagcaacaacagcaacagctgggacaccagatcttcagcagcacagttgcacccacctcgatcagcagtgcgactgcggtggcaggcggcggcgggggctatggacagttcaatgccaccgccatcagcacgcccacctcgttcgccacggccggcagtgtggtcagcagcagcgccaaaggtccgacgaagggtcagaaggggcagcagcagcagcagcagcatcagcagttccagcactaccagagcagcagtcccctgatagcggacagtcccatcccgagtcccagcggcgccattgcagcggcggcgattaagcctccaaccccgcagccccaacaaatgcagatgctgccccagcagttcaccatctcccagcagccgcagcagcagcagcaggcgcagcaggtcttccagtttatccagggaccgcagggccagctcatagccaccactccccagcaacagcagcctatccagcagcagcaacagcagcagcctcagccagtccagcagcagcagcagcatcgattcgttagcaatgccggagtcacgggaatgtccaccggcaagaccggcaaggcaccgcagcagatactgcccaagccgcaacaggagctccagcagcagaagaagggcacgaacggcggagcacagatctcgcaatcggcgcagcaaactggccaagccaacaatcccacccaacagcagcaacaacaacaacagcaacagatcctgctacctgccaccaccaaccagccgcaacagttgctccttaaccaaatgcccgtgctggtgcaacagaatccgcagggcgtgcagctaattctgcgaccacctacgcctcagctgacgaccacaccgtccctcgtcatccagcagaatgcccgcgggcaaccccaactgcagacgcagccggcacagcaacaactgctgcggatagtgggcaccaatggagcgacaatgcagctggctgccgcgcccaccttcattgtgtcctcgcaggcgaacctcatccatcagacggcggcaggtcagttccagagcgccatcaagtctggcacccagctcacgggtctgcatgcggctctagcacaggcacaggcccaggcgcaggcgccacgatctcagccgtttgccacgacggccacgttgaacacccagctccttggccagagtgtagccgcccagctgcagaacttacagctggctgcggcccaaattcagatgcccaacggcctcaccgccatatcccaactgccagctcatttgcagcagagcctaggtggagccaccatcaatctgaatcagctgaatggggcgcatatccagcaaatagctaacgcttttcaggcgccacaggctccaggaacgaatagcggaggagctaacagcacgacggagctctttacacaatcctcgccggcacatgtgccgctggccgtcaccccggagccattgcgtcagcccacgcccgtgcccatgatgcagcaacaacaacaggctgcgatggccaccattcagttgcagcagcagcagcaacaacagcagcaggcgcagatacagcagctgcagcatcaactgcagcaaacacagtcccaggtgcagcaagcccagcaatctgtccaggcgacaactataccggagcccaagaagaaaccgcgaccccgcaaaaagaagcaacctccagctccaactccaccagcagcctctccgacagtagcacccgctgaagtgagacccccgacgccacaaaagatagccatcgccgccacgcccgcgccacagccaactgtacgcgccaagtctccatcgccacccccaacgacgatccagcgaagtagcaatggaaagctggatctcggcgatgtgatgaagttttgcggtatcaccggagacgatgacgacgacgaggactatggaatgggcctagaaacagggctagcatcagaatcagagccagctcaagcccacgcgcctgccacagggagtgctgcagcgacgacgggcagcagcggcgacatcatgatctccataccgaaccaaaatggcagcgatggcctgccttttaccttgaccatacccgctccgcagccccagtcatcgacgggatctcaggcggcgggaaacgaatcagccaccgaaataccaaatatcctcatcaaaatcgatccaagtgccgaggcagcggtgccgccatttggtctatccacgccacggttgcccacagcagaggaaattcagaagcaggcgcagcaacatttggcacaacaggcggccgctgcagcagcggcggcaaaggcaaccacagccacgcccaccataaacattagcctgcctagcatgacattgcagccgcaggtagcgccggcaccagttgtgacgccaactcctgccccatcctcgaaccaggtgtccacgacagttgttgtcaaaccaagaaggaagcccgcggtgcgaaggaatgccaagaaacctgacgtgaccagcagcgcaagcaatatgatctcctcgagcagtggcactaccactacaatcagcagcagcactagtaccatcctaaacaacagtcaacccaccactgtaaacaccatcactttaaccacgcccacgccagtgacaagcagcagcaattccaccctgatgctgacccacggaactccgacagcagtgcccagtcagattggcaacattcagatctcacaggtgcataatcatcacccatccgcattgccggtgagcagtgcggtggagaacaagatccagataatgccgatcctgccaccgggagccacagtgatgggcggaacaccaggaaccggaggtcatgcccccaccacccaatcaacccagttggtctttcagccagcggcaccatcggtggcgcctacgattacttcagactccactgggtttaagctctccagcgatggtcggcaaatgcagctagtggccataccacaggcgactcctccatcgccagcagccagttctgcccagccaccggttcccacgcccactcctgcgttaactgccggaggagctacaattctgccacctcagttgacaggaatgcccgccaatgtatcggtttctgtgggatcccctgcctcggctgcggctctggtgccacaactcactggaagtctcacactcaccgtgtcggagcagtgcgagcgattgattctgcgacatgatccgaacaatccccaggaccatcaatcgcagctcatcctgcaggctctgctcaagggcgctctgcccaatgttaccataatcaatgagccgacgcgcgtggatccaaataaacagacaactccgatgttgcagccgcaacagcaagttatccaaataccccagccactaactcaaccccagcaaattctgcagcagcaaccaaaggtttcaacagctcccaaaatagaagttaaagtggctaacaacagaaagttgtcaggtcaggtaacacatcctgccagcagtatgttaggaatgaccagcactacaactaccatgtcaacgatgaaaccaccggccaatctacccgccaagcagaacgaagtcgtatctttcccgcagctaccactgaactcgcaggttctcattcagcagcagcctatgatttctgcgcagctacagcctcagttgcaacccgtcactgtgcccgttagcttgcccactcagccggtaccgattccatctccggcgcctgctccacaactggccaacaatggccagcagcggtacattgcactgccgcccattgatcccaccacccagcagttgttctgcctgaacagtgtcacgaatcagataactgcgatgagtgcgggccagacggctgcttcgattggtcccaccgagcggttgcttattgctccggctggtatcaatgctcagcagctggctcaatgtctgcagttggggcaacttcatttcaatgatgtgaatccgctgccggctcagcagcaacagcagcaggtggctaccacctcgtcttcgatgacactttcaatgccactgcgtcctcagccaccgcagcaacaaatagtgcatcccatccagccaatcacaaatggactggccatcaccacaacggcaagtagcacgctgaccaccaccactagcaccacttcgttaacaacggtgaccaagcaggtggccaatgtggcgccgattatagatcaaagcaaagcgaagctggagcccgctaaggcagccaccagtggagccggtggcggagcggtggtcaaaaagaagccggtgaggaagcccaaggcaacgccgaccaatgcctccgaactggccaatctaaagaacgccagcgtagtcaagccaatgccaaagctcgatcccctatcgcaaaagcccaacaataacccggtgcagatcgtgcagcctaatgttgccagtggaaagcagatgattgtggtgggcagctccagttgcacaacaactaccacgacaacgatgagcgccagcatccagcccttccagacaacgagtggcaccaaattggtgggcgttacggtgcccatgcagcagcaacaacccactggcacggcagctatattgcaacagcagcagcaacagaggacgagcttcagcttgacggcaaccacagtggcgacacctgctccgatgccatctccaacggtagcgtctggggtcagccaactccaaatgcagcaacttccatcccagcaggcgcagcaacagttgcagccgcagcagcagcaacagttgcacgctcctaagcaacaacaaccgcaaccaaatactgtgtcccgggtgcaaaccatccaacttacgcctcagatgcaacagatcttcaagcaggtccaaatgcaaattcagttcctcactattaagttacaaaataaatcaaccttcttgcccgtctcgccggaaattgattcggcaactattgccgcctacaacaaaccgatgacagatgcggagataaatctggctctgcagcgtctcttcgcagagcagcaccggattctggcctccggcaaagaggtacccactccagagggtatgttgccaacagcaaatggaacgggcggcttcagtctcatgccacaaccagttcaacaacaacaacagcagcagcagcaacagttgcaatcaactgttcctgagccactgaaaacaatcgttccggccaatgtggttcagccaaacaaccactcatccactcctgcaggagcggccactagtggtgccacaactgctgccaatatccagcagaatataacacagaggatacacatctaccctatgcagcaccaacaacaacaacagcagcaacaacagcagcagcaacaacagccgcagcatcagcaacaacagccccagcatcagctacaacagaccggagccaaaccaaagcaggcacaaccaaagccgccgccacagcaggagcaacaatcgcaactccaaataaaacccaaggaaccggccaacaggagcaaagtgactagccagcagctgcagcaaaatcctcagccacccaatggaaccatcaatatgttgccacaaaaagttagcatgatgccggttgtccagcagcaacagcagcagcagccgcaacaacatcagcagcaaccaatgctcaacaaaagcgggccacctccactgatcttcgccagctctacaaatatgctgagcaacagcaatagcaacaacctccacaacagcaattctgcaatgcaagtaaacaacatgttgcccctgccaccattgcaaccccaacagcagcctcagccacagttacagcagccaccaactccaatcctgccgccagtagcaccaacaatggccatgggagtggcgccaggaccgatgggaaatctagtgccatccctcccgaccataccgagcctgcccctgtatatgccgagcaaaatggacgaaatgccaacacagaaaccaaaaattgcacgcctctccttgttcgttcgccaactggaggtcgatcaggagagctgtctgaagccggattacgtgaagcctttccgcacaaaggatgaggcggttaaaaggctaatcaggtatcattgcatgcatgagaacgacgttgagttgccttctgacgaagacgaggaatttgagtccactgctctggaattccaggacaaattccgccaactgaacggcaagttccaggaaattctgatgcaggagtcaacgctgccacaccgaacttctgagttgctacaaatgcagcagctgatgatcgatgatctcaaaggcgagatcaatgagattcgaaccgctgaaaaggagttagagcagcagctaaaggaggagcagactagcgaaaaatcgacggcggaaagcgatgtactttcggaggcgaaagttaaggaggagattaaacaggaaccaatagataagtctgctcagccagacggcgttgttgacaaatttgatttgctgaaaaacagcgctgatgtcaataaagcattcagcaagtcacagccacagcagactactacagtaaagcaagaggagagcaacgaatctggggaaccttctgtaaatggatctgtaaagagcgaaggccacgataaggagtcgtcgaaatacattaagaaggactacgacaacttcgacattgagtctgagctcaccactagcttcatcatgaagaaggtggagaacaaagcggctttggcaaagagcgctgagaatcgctaccaggaaagaaatatgatggaccaacaacagcagcagcaacatatcaaagccaatggaggagatcccgtagaccaggatgatggctggtactgcctgcaaaaggagcttaacctgatgaataacgatcatatgcagcaatcgcagcaacaactaaatggaaaccaacagcatctgcccatcaatcgtcaaccggcgccacagcaatcacacttcctggacaattcaaattcgaacaacatgatgggcaatagcaaccacatgtcggatctgttccccaatggttgcattgacaagagcaatcaaagcaccaccagcagcagcaatagcagttgcgttagcgaaagccttcctggttctaatcccgtgaagactgtctcctcatgcagcggccagcgcgaacagccggtggtcatggatgctgatcagcaaaacgagcatcccgccatcagtgagttctttagcagcgattcggacgtccagaaatccgtggagacacgactcgaggcaatgttcggggagtcgcccgtccatttggacgttaagagcagcaacgatgccaacgacattgagtccaacctggacgagatttttggcgactcgaaatcgccagctgccaaccacaaaagtaaaatgagcatgtgggtgccagacgcatcatttatgcagcaggcgccgcagcaacagacgtcgcaacagcagcaacaatacgcagggccacaacaaccatcacagcagcaacatcacatcgaactgaactgcaacaacaatccgcgctggatgcaaagcatggaggcgcactacagcgactttctttccaccgggactagcaatggagagctggtgaacggagaatcgcgaaaaagaagctgggatagccagctcatgggctctggcagtgatatggaagatgacaacagcagcaagcgtctgtgcacggcttcatcgtcctcctcttcgtcgcccatgtcgtcgcagcagcagcacgttcaggtggcacagcatggtctcttggactcggacatgttcccccagatgttgatggatcagcagccgcatcagcaacaacagcaggtacagcagcaacacaactttgcctacgccaacataatggatcaacagcagcagcaacagcaccagcagcaacatttccagcataacctgcagcaacagatacaacagcagcagcagatgcatgttagccacatgggcggagcagcagtagtggccggcggagagtacgacgatgacatcagtcggcatgtggccagcgccatcgacagcatcctgaacttgcagaacagcggcgactcgctgcagttctccctgggttcgatcctcggtgacagcatgctggacgatcaacggcaagcaggagccaatttgcccagctgccagcaggagcaggcgatccaacgacgccgccacctgggcgaggagatgaacgactgcctgattagcggcggtggttccgcggttagcggagtggcggacaacagtagtaacatactgctagagcaccaccaccagcagcatcagttgatgcagagccaccaacagcagccgcatctgcagcatcaccaccatcagaacatgtcgcaggcccaggcgcagcacatgggacagctgaacgactttagctgtgtagccggtggcttggacgatcctgtcaagtcgataatgacgtcctgacttggagccccaagcagcacatcagcggcggaaaatgcttcgaacagtctgatactagagtttaatgccaccaccttgtgaaaaacggatgataataatgacttcctgattgtatagatacccgtcttagttgtccttggttattgttttacccccgagtacaacctttaattagatctaagttgaatccgtgtagcataaattgtttttacttattgtttgtattgtttagttcaatgatttgtaccatatttgaacagccctcagataaccaattacttaatgcaaggggacagaacgaagagagcgaggtgaaagtaatggcatctacatacaatacggctgagccccaataaagctacgaaagcaattgacgtggtgaattgaaaactaaacgacgaatagcatcaaatgcttctcacagaggtacactaaaatggaacataatataaaaaattatatagctacagtaactaagagaaagcccctagacaatttatacaaatgaaaccaagaaagtacgtaagataaagggaagaaggtaacgatgggaataaacacgcgcaaagtgagagaaatgaaaggaaaacgaattggttgtataacttataattaagttacttgaagcaattctgagaaacaattaaaatcacgttgccaattttgttgtcgtttgaatatcaggcagagcaatttatccgcatccaatcgatttttaaaagcccagataaaatatatttatttacgcactacgttgcgaaggagttttgtaaatagttagaacatgcagcagtttcagtatatataaatatatagctaagcatttttaaaaggatatcatttaaagcgcttcaagtacgactgcttctgatattacagacaaaccaacaaaccaaccaaccaaccaatcaacctattgacgtgctcaatatgagagtaaagaatatttcggcatagctacgggtgtcattggaaagcaaatgcaaagatcttagggattaacgagtaccaactatggcaaactacgaagcaaaccaaaagcagttcaaaaatcacaattttagccaatttagtttaaacatttataaattataaattattgaatctaatgtaatatcttatacacttaaaccgatcgattgaaacgagaagctagcagttctgagatgaggaaccccaggaaaacaaataacaacaaattaacgaatcgtatcaattgtatctcgatcttgcgacaaatctgacttgtgatttgtgccacgttttaaatatagatgtctgattagcttgtaaacatacataataaatacgattaaaaagtctatacaaaagcccacactcagtaagccactacgaagtttatccaaaatgaatcgcgactcgcggtagcaattgtaaatgtttgacgtggggagggcgggcgccacgcatcaattacataccaattacaatactaactactactttaataccaccactaacacacctactacgatacgtactaccgtggacggattgcttgtattacgtgtagtctgtaagggaaattcgagaggtaccatattttaagatgtgcttcgtttgttcaagtccacccatccatgacccatgacccatgaaccctgaccctgaactcctgtaccaaaacccgaatcataaccataaccatccactcccacaaaagcaaagtctcccacctgcctagcgtcccgtgtgagttgcatgatcttgtatttctatttggaaactttctagttattattcaaagacaaccactttcgtgtaaaaatgtatatgtaatagcataatttaagttggcaacccaaaactgaataaacctaattctactgatagataactaatgcgaattgttttaagttgtatttagaacgaaatcgaaatcctcagaactcaaatcggtcgcccctcccgcaaccaactttccacaacaacaaagcgcagtgtcgtcacttgaattgttcagctaaatgtgtctaaatgtaattaaacataaagagaaaactaaagatttttaaacaaacattgtgagttttattaaacatcatcagcatagggagaggagaggacagcagcaaagcaaaggaaaccaacatctttaatatttgatagtttattaagcccatccattcatagtcgtcataaacattaagtacataaaagaaaaacccaacaaaagattcaatataaatctcactgaaacgcaacaaacaaccgaaaacccaact

SEQ ID NO: 6 - DNA, 3921 bp, Drosophila melanogaster
gctcattaacgtttcgcttggctaagtgcgcacctcgtcactgaaaccagttagcaaaagaaatttttgttcaagcttaaaccgatacctacgtcactattcgatcattttgaatgcaaattattcccagattaataaactaggtcgccatccgtggcgctgccgccgggctgcggacccgtactttcgacggaaaatatcgggaccattttcataaacgatctatgatacagttttaaaacactttaacgctttccatcgtgcacacttaaaacaaaactcataaagtgaaagttcatatcctggcaagtaaaaagtgataaatgttaaaacgtctgaagtgatagtctggagtttgattaccaggtgaagaagataatatatatgggatgtacaaagttggcaacgaagccccccttctaacaacaatgaaaaagggagtaatcctactttctcactttcaataataaacatacgcacgtcagccaccgtcgaagtctccaacgccgcattaaaaataattaattatgatgctaacaactgaacacataatgcatgggcatccccactcgtcagtcgggcagagtactctatttgggtgctccacggcgggccatagcggaataaatcagctgggcggcgtatatgttaatggccggccactgcccgattcaacgcgtcaaaaaattgtcgaattggctcattccggcgcacgtccttgtgatatttcaagaatactacaagtgtccaacggttgcgtaagcaaaattttgggcagatattatgaaactggatcgataaaacctcgagctataggtggttcaaagccacgagtagctacaaccccggttgtgcaaaaaattgcagattacaaacgggaatgtcccagcatatttgcgtgggaaatacgagatcgactgctatcggaacaagtttgcaatagtgataacattccaagtgtttcatctattaatcgagtcttacgtaacctggcctcacaaaaggagcagcaagctcagcaacaaaacgaatccgtttatgaaaagcttcgcatgtttaatggccaaacgggcggatgggcatggtatccaagcaatacaacgacggcacatttgacgctaccaccagcagcttccgttgtgacatctcctgcaaatttatcaggacaggccgatcgggatgatgttcaaaaaagagaattacaattttcagtagaagtttcgcatacaaactctcacgatagtacatcggatggaaactctgaacataattcatccggggacgaagactctcaaatgcggttgcgcctaaaaaggaagttacagcgcaatcggacatcattttctaatgagcaaattgacagtcttgaaaaagaatttgaaagaacacattatcccgatgtttttgcgcgagaaaggcttgctgataaaattggtttgccagaggcacgtattcaggtttggttttcaaaccgacgagctaaatggcgccgagaagaaaaaatgcgaactcagagacgatcggccgataccgtggacggcagtggtcgaaccagcacggcaaataatccttcaggaacaactgcatcttcctccgtcgcaacgtcaaacaactcgactccagggattgtgaactcagcaatcaacgttgcggaacgaacatcatctgcattagttagtaatagccttcccgaggcttcaaatggaccaactgttttgggtggtgaagctaatactacacacaccagctctgaaagcccaccccttcagccagcggcaccgcggctacccttaaattctggattcaacaccatgtactcatctattccacaaccgattgcaacgatggctgaaaattacaactcctcattaggatcaatgaccccgtcatgcttacaacaacgcgatgcctatccttacatgtttcacgatccgttatcactaggatctccctatgtgtcagcccaccatcgaaacacagcttgcaacccctcagctgcgcaccaacagccccctcagcatggcgtttataccaatagttctccaatgccatcatcaaacacaggtgtcatttctgcgggcgtttcggtgcctgtccagatttcaacgcaaaatgtatctgacctaacgggaagcaattactggccacgtcttcagtgatcgtcaatctttggctcaccattagatcatttgtcaaaggcgactgccgctgcaatcattgccgcacaagcagctgagaaaagccataaacaccgaaaagagcattcaattgttagtatacacaccacaaaaagaaagaaaaacccacctagtttgaagagacagacaacgtgctgttcatccagatatttaaacatgaccttcggggcggggaaggatgtagatgttgtcataggccaaggtcttatagtgtccttaacaccacatatttcaggaaagccggagctttttggtagtcctaacgttaactaccatcaggactttcaagtaatttaaataggctgaaccttttcggtctgaaacttgtgaattgcacactatatgtattttaaaaggcaaatgaaatgttaaatctacattaaaattgatgttatttttgaataaaaattcaagatttgtaaacaatatttccgcatctttcgcatccttttttaagttccaaacattcctgatctgctaagataggttaaagtatttcggggaatcttcaactttactgttgataagcacgcaatcgggatattagacttctgcagttgcttgcaattttttaagccttcacaattatatttatattttgaaccattttttgatattataaattaaacttattcatgtattaatactatttattaatcgtctttaagtaaagaatgagcaattatgctcatacatacttaaaaaaaaaaccatgtgttaccaaacttagggaagtgcgtggaagacggatataaagcaaagtgacgttatacatgattttacatttacattaaattcttatatgacaatttatgtttttagagtcgtaggcgttatatagtttcttctactagtaacgtacttctaaacaaatctctcatacccttttcctctacgattatatcaccaaattgttttgtttcccaaatataaccattgcattttgaattaaaaattacatttttttaagttcaataaaaactaccagataatgctaattaaaggtcttataaacattttgacgaaattatgactttgaattatatatatacatttaagtacacgaacaatttctactgtacgtaactattaaaagatatagtgtttctaatttgatcggcatgaaataaactaaaaccactgattcatttgctagaaaacccataaacctcataaaaataaataaaaaacatttcgcgcatactggtcgatatgttttaaaaattgtatattttattataatgtattcaaattataaattatctgtacatgattcttttaaatgtaagctattagttattaatccatgtgtacaccgtttgtaaaacagaaacttataacaacgtaagattgaatgaattgcaacgaacttgactccaatgaaggaaatgagctaacatctcatccagataaacaccaaccccaacaagccttcaggtttatatcataataataaaatgtcgtatatgtataatgtatggttcaaatttatatttagaataaattaatgtaacatttcagaatatacaggtgtatctgttgttcttaaaaggggcttaatgaaatccaactttaataaaaaacaagagagaatgctatagtcgagttcgtcgactattagatacccgttactcagctagtgtgtgaccgttttggctgcaaaaaacgatagaaatttacacaactaataaaatttaaaaaaaaaatattcaaaaaaaatttc

SEQ ID NO: 7 - DNA, 3684 bp, Homo sapiens
acactttaatatcaacctgtttcctcctcctccttctcctcctcctccgtgacctcctcctcctctttctcctgagaaacttcgccccagcggtgcggagcgccgctgcgcagccggggagggacgcaggcaggcggcgggcagcgggaggcggcagcccggtgcggtccccgcggctctcggcggagccccgcgcccgccgcgccatggcccgaagaccccggcacagcatatatagcagtgacgaggatgatgaggactttgagatgtgtgaccatgactatgatgggctgcttcccaagtctggaaagcgtcacttggggaaaacaaggtggacccgggaagaggatgaaaaactgaagaagctggtggaacagaatggaacagatgactggaaagttattgccaattatctcccgaatcgaacagatgtgcagtgccagcaccgatggcagaaagtactaaaccctgagctcatcaagggtccttggaccaaagaagaagatcagagagtgatagagcttgtacagaaatacggtccgaaacgttggtctgttattgccaagcacttaaaggggagaattggaaaacaatgtagggagaggtggcataaccacttgaatccagaagttaagaaaacctcctggacagaagaggaagacagaattatttaccaggcacacaagagactggggaacagatgggcagaaatcgcaaagctactgcctggacgaactgataatgctatcaagaaccactggaattctacaatgcgtcggaaggtcgaacaggaaggttatctgcaggagtcttcaaaagccagccagccagcagtggccacaagcttccagaagaacagtcatttgatgggttttgctcaggctccgcctacagctcaactccctgccactggccagcccactgttaacaacgactattcctattaccacatttctgaagcacaaaatgtctccagtcatgttccataccctgtagcgttacatgtaaatatagtcaatgtccctcagccagctgccgcagccattcagagacactataatgatgaagaccctgagaaggaaaagcgaataaaggaattagaattgctcctaatgtcaaccgagaatgagctaaaaggacagcaggtgctaccaacacagaaccacacatgcagctaccccgggtggcacagcaccaccattgccgaccacaccagacctcatggagacagtgcacctgtttcctgtttgggagaacaccactccactccatctctgccagcggatcctggctccctacctgaagaaagcgcctcgccagcaaggtgcatgatcgtccaccagggcaccattctggataatgttaagaacctcttagaatttgcagaaacactccaatttatagattctgattcttcatcatggtgtgatctcagcagttttgaattctttgaagaagcagatttttcacctagccaacatcacacaggcaaagccctacagcttcagcaaagagagggcaatgggactaaacctgcaggagaacctagcccaagggtgaacaaacgtatgttgagtgagagttcacttgacccacccaaggtcttacctcctgcaaggcacagcacaattccactggtcatccttcgaaaaaaacggggccaggccagccccttagccactggagactgtagctccttcatatttgctgacgtcagcagttcaactcccaagcgttcccctgtcaaaagcctacccttctctccctcgcagttcttaaacacttccagtaaccatgaaaactcagacttggaaatgccttctttaacttccacccccctcattggtcacaaattgactgttacaacaccatttcatagagaccagactgtgaaaactcaaaaggaaaatactgtttttagaaccccagctatcaaaaggtcaatcttagaaagctctccaagaactcctacaccattcaaacatgcacttgcagctcaagaaattaaatacggtcccctgaagatgctacctcagacaccctctcatctagtagaagatctgcaggatgtgatcaaacaggaatctgatgaatctggaattgttgctgagtttcaagaaaatggaccacccttactgaagaaaatcaaacaagaggtggaatctccaactgataaatcaggaaacttcttctgctcacaccactgggaaggggacagtctgaatacccaactgttcacgcagacctcgcctgtggcagatgcaccgaatattcttacaagctccgttttaatggcaccagcatcagaagatgaagacaatgttctcaaagcatttacagtacctaaaaacaggtccctggcgagccccttgcagccttgtagcagtacctgggaacctgcatcctgtggaaagatggaggagcagatgacatcttccagtcaagctcgtaaatacgtgaatgcattctcagcccggacgctggtcatgtgagacatttccagaaaagcattatggttttcagaacacttcaagttgacttgggatatatcattcctcaacatgaaacttttcatgaatgggagaagaacctatttttgttgtggtacaacagttgagagcagcaccaagtgcatttagttgaatgaagtcttcttggatttcacccaactaaaaggatttttaaaaataaataacagtcttacctaaattattaggtaatgaattgtagccagttgttaatatcttaatgcagatttttttaaaaaaaacataaaatgatttatctgtattttaaaggatccaacagatcagtattttttcctgtgatgggttttttgaaatttgacacattaaaaggtactccagtatttcacttttctcgatcactaaacatatgcatatatttttaaaaatcagtaaaagcattactctaagtgtagacttaataccatgtgacatttaatccagattgtaaatgctcatttatggttaatgacattgaaggtacatttattgtaccaaaccattttatgagttttctgttagcttgctttaaaaattattactgtaagaaatagttttataaaaaattatatttttattcagtaatttaattttgtaaatgccaaatgaaaaacgttttttgctgctatggtcttagcctgtagacatgctgctagtatcagaggggcagtagagcttggacagaaagaaaagaaacttggtgttaggtaattgactatgcactagtatttcagactttttaattttatatatatatacattttttttccttctgcaatacatttgaaaacttgtttgggagactctgcattttttattgtggtttttttgttattgttggtttatacaagcatgcgttgcacttcttttttgggagatgtgtgttgttgatgttctatgttttgttttgagtgtagcctgactgttttataatttgggagttctgcatttgatccgcatcccctgtggtttctaagtgtatggtctcagaactgttgcatggatcctgtgtttgcaactggggagacagaaactgtggttgatagccagtcactgccttaagaacatttgatgcaagatggccagcactgaacttttgagatatgacggtgtacttactgccttgtagcaaaataaagatgtgcccttattttaccta

SEQ ID NO: 8 - protein, 422 aa, Homo sapiens [sequence omitted]

SEQ ID NO: 9 - protein, 436 aa, Homo sapiens
MQNSHSGVNQLGGVFVNGRPLPDSTRQKIVELAHSGARPCDISRILQTHADAKVQVLDNQNVSNGCVSKILGRYYETGSIRPRAIGGSKPRVATPEVVSKIAQYKRECPSIFAWEIRDRLLSEGVCTNDNIPSVSSINRVLRNLASEKQQMGADGMYDKLRMLNGQTGSWGTRPGWYPGTSVPGQPTQDGCQQQEGGGENTNSISSNGEDSDEAQMRLQLKRKLQRNRTSFTQEQIEALEKEFERTHYPDVFARERLAAKIDLPEARIQVWFSNRRAKWRREEKLRNQRRQASNTPSHIPISSSFSTSVYQPIPQPTTPVSSFTSGSMLGRTDTALTNTYSALPPMPSFTMANNLPMQPPVPSQTSSYSCMLPTSPSVNGRSYDTYTPPHMQTHMNSQPMGTSGTTSTGLISPGVSVPVQVPGSEPDMSQYWPRLQ

SEQ ID NO: 10 - protein, 436 aa, Homo sapiens; identical to SEQ ID NO: 9

SEQ ID NO: 11 - protein, 422 aa, Homo sapiens [sequence omitted]

SEQ ID NO: 12 - protein, 3003 aa, Drosophila melanogaster
  misc_feature (2977)..(2977): Xaa can be any naturally occurring amino acid
MSRRQSLDRPGILSPPGPFLTPSPSPSISASPRLQPSPSLSPFPQDVVLVAGGSQVNANSNPQEHERKAATPGRRQSIHQFTNGSPNAGTVVFQPSPSQSPAAATTVIGVTSGGLIATAAGGTGAGLSTGVGAGSSSGVGIGVALHGNAIGNELNATLVGSNNIQLNKKGTKRATQQQQQQQQQLGHQIFSSTVAPTSISSATAVAGGGGGYGQFNATAISTPTSFATAGSVVSSSAKGPTKGQKGQQQQQQHQQFQHYQSSSPLIADSPIPSPSGAIAAAAIKPPTPQPQQMQMLPQQFTISQQPQQQQQAQQVFQFIQGPQGQLIATTPQQQQPIQQQQQQQPQPVQQQQQHRFVSNAGVTGMSTGKTGKAPQQILPKPQQELQQQKKGTNGGAQISQSAQQTGQANNPTQQQQQQQQQQILLPATTNQPQQLLLNQMPVLVQQNPQGVQLILRPPTPQLTTTPSLVIQQNARGQPQLQTQPAQQQLLRIVGTNGATMQLAAAPTFIVSSQANLIHQTAAGQFQSAIKSGTQLTGLHAALAQAQAQAQAPRSQPFATTATLNTQLLGQSVAAQLQNLQLAAAQIQMPNGLTAISQLPAHLQQSLGGATINLNQLNGAHIQQIANAFQAPQAPGTNSGGANSTTELFTQSSPAHVPLAVTPEPLRQPTPVPMMQQQQQAAMATIQLQQQQQQQQQAQIQQLQHQLQQTQSQVQQAQQSVQATTIPEPKKKPRPRKKKQPPAPTPPAASPTVAPAEVRPPTPQKIAIAATPAPQPTVRAKSPSPPPTTIQRSSNGKLDLGDVMKFCGITGDDDDDEDYGMGLETGLASESEPAQAHAPATGSAAATTGSSGDIMISIPNQNGSDGLPFTLTIPAPQPQSSTGSQAAGNESATEIPNILIKIDPSAEAAVPPFGLSTPRLPTAEEIQKQAQQHLAQQAAAAAAAAKATTATPTINISLPSMTLQPQVAPAPVVTPTPAPSSNQVSTTVVVKPRRKPAVRRNAKKPDVTSSASNMISSSSGTTTTISSSTSTILNNSQPTTVNTITLTTPTPVTSSSNSTLMLTHGTPTAVPSQIGNIQISQVHNHHPSALPVSSAVENKIQIMPILPPGATVMGGTPGTGGHAPTTQSTQLVFQPAAPSVAPTITSDSTGFKLSSDGRQMQLVAIPQATPPSPAASSAQPPVPTPTPALTAGGATILPPQLTGMPANVSVSVGSPASAAALVPQLTGSLTLTVSEQCERLILRHDPNNPQDHQSQLILQALLKGALPNVTIINEPTRVDPNKQTTPMLQPQQQVIQIPQPLTQPQQILQQQPKVSTAPKIEVKVANNRKLSGQVTHPASSMLGMTSTTTTMSTMKPPANLPAKQNEVVSFPQLPLNSQVLIQQQPMISAQLQPQLQPVTVPVSLPTQPVPIPSPAPAPQLANNGQQRYIALPPIDPTTQQLFCLNSVTNQITAMSAGQTAASIGPTERLLIAPAGINAQQLAQCLQLGQLHFNDVNPLPAQQQQQQVATTSSSMTLSMPLRPQPPQQQIVHPIQPITNGLAITTTASSTLTTTTSTTSLTTVTKQVANVAPIIDQSKAKLEPAKAATSGAGGGAVVKKKPVRKPKATPTNASELANLKNASVVKPMPKLDPLSQKPNNNPVQIVQPNVASGKQMIVVGSSSCTTTTTTTMSASIQPFQTTSGTKLVGVTVPMQQQQPTGTAAILQQQQQQRTSFSLTATTVATPAPMPSPTVASGVSQLQMQQLPSQQAQQQLQPQQQQQLHAPKQQQPQPNTVSRVQTIQLTPQMQQIFKQVQMQIQFLTIKLQNKSTFLPVSPEIDSATIAAYNKPMTDAEINLALQRLFAEQHRILASGKEVPTPEGMLPTANGTGGFSLMPQPVQQQQQQQQQQLQSTVPEPLKTIVPANVVQPNNHSSTPAGAATSGATTAANIQQNITQRIHIYPMQHQQQQQQQQQQQQQQPQHQQQQPQHQLQQTGAKPKQAQPKPPPQQEQQSQLQIKPKEPANRSKVTSQQLQQNPQPPNGTINMLPQKVSMMPVVQQQQQQQPQQHQQQPMLNKSGPPPLIFASSTNMLSNSNSNNLHNSNSAMQVNNMLPLPPLQPQQQPQPQLQQPPTPILPPVAPTMAMGVAPGPMGNLVPSLPTIPSLPLYMPSKMDEMPTQKPKIARLSLFVRQLEVDQESCLKPDYVKPFRTKDEAVKRLIRYHCMHENDVELPSDEDEEFESTALEFQDKFRQLNGKFQEILMQESTLPHRTSELLQMQQLMIDDLKGEINEIRTAEKELEQQLKEEQTSEKSTAESDVLSEAKVKEEIKQEPIDKSAQPDGVVDKFDLLKNSADVNKAFSKSQPQQTTTVKQEESNESGEPSVNGSVKSEGHDKESSKYIKKDYDNFDIESELTTSFIMKKVENKAALAKSAENRYQERNMMDQQQQQQHIKANGGDPVDQDDGWYCLQKELNLMNNDHMQQSQQQLNGNQQHLPINRQPAPQQSHFLDNSNSNNMMGNSNHMSDLFPNGCIDKSNQSTTSSSNSSCVSESLPGSNPVKTVSSCSGQREQPVVMDADQQNEHPAISEFFSSDSDVQKSVETRLEAMFGESPVHLDVKSSNDANDIESNLDEIFGDSKSPAANHKSKMSMWVPDASFMQQAPQQQTSQQQQQYAGPQQPSQQQHHIELNCNNNPRWMQSMEAHYSDFLSTGTSNGELVNGESRKRSWDSQLMGSGSDMEDDNSSKRLCTASSSSSSSPMSSQQQHVQVAQHGLLDSDMFPQMLMDQQPHQQQQQVQQQHNFAYANIMDQQQQQQHQQQHFQHNLQQQIQQQQQMHVSHMGGAAVVAGGEYDDDISRHVASAIDSILNLQNSGDSLQFSLGSILGDSMLDDQRQAGANLPSCQQEQAIQRRRHLGEEMNDCLISGGGSAVSGVADNSSNILLEHHHQQHQLMQSHQQQPHLQHHHHQNMSQAQAQHMGQLNDFSCVAGGLDDPVKSIMTSXLGAPSSTSAAENASNSLILEFNATTL

SEQ ID NO: 13 - protein, 655 aa, Drosophila melanogaster
  misc_feature (544)..(544): Xaa can be any naturally occurring amino acid
MMLTTEHIMHGHPHSSVGQSTLFGCSTAGHSGINQLGGVYVNGRPLPDSTRQKIVELAHSGARPCDISRILQVSNGCVSKILGRYYETGSIKPRAIGGSKPRVATTPVVQKIADYKRECPSIFAWEIRDRLLSEQVCNSDNIPSVSSINRVLRNLASQKEQQAQQQNESVYEKLRMFNGQTGGWAWYPSNTTTAHLTLPPAASVVTSPANLSGQADRDDVQKRELQFSVEVSHTNSHDSTSDGNSEHNSSGDEDSQMRLRLKRKLQRNRTSFSNEQIDSLEKEFERTHYPDVFARERLADKIGLPEARIQVWFSNRRAKWRREEKMRTQRRSADTVDGSGRTSTANNPSGTTASSSVATSNNSTPGIVNSAINVAERTSSALVSNSLPEASNGPTVLGGEANTTHTSSESPPLQPAAPRLPLNSGFNTMYSSIPQPIATMAENYNSSLGSMTPSCLQQRDAYPYMFHDPLSLGSPYVSAHHRNTACNPSAAHQQPPQHGVYTNSSPMPSSNTGVISAGVSVPVQISTQNVSDLTGSNYWPRLQXSSIFGSPLDHLSKATAAAIIAAQAAEKSHKHRKEHSIVSIHTTKRKKNPPSLKRQTTCCSSRYLNMTFGAGKDVDVVIGQGLIVSLTPHISGKPELFGSPNVNYHQDFQVI

SEQ ID NO: 14 - protein, 761 aa, Homo sapiens
MARRPRHSIYSSDEDDEDFEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTDNAIKNHWNSTMRRKVEQEGYLQESSKASQPAVATSFQKNSHLMGFAQAPPTAQLPATGQPTVNNDYSYYHISEAQNVSSHVPYPVALHVNIVNVPQPAAAAIQRHYNDEDPEKEKRIKELELLLMSTENELKGQQVLPTQNHTCSYPGWHSTTIADHTRPHGDSAPVSCLGEHHSTPSLPADPGSLPEESASPARCMIVHQGTILDNVKNLLEFAETLQFIDSDSSSWCDLSSFEFFEEADFSPSQHHTGKALQLQQREGNGTKPAGEPSPRVNKRMLSESSLDPPKVLPPARHSTIPLVILRKKRGQASPLATGDCSSFIFADVSSSTPKRSPVKSLPFSPSQFLNTSSNHENSDLEMPSLTSTPLIGHKLTVTTPFHRDQTVKTQKENTVFRTPAIKRSILESSPRTPTPFKHALAAQEIKYGPLKMLPQTPSHLVEDLQDVIKQESDESGIVAEFQENGPPLLKKIKQEVESPTDKSGNFFCSHHWEGDSLNTQLFTQTSPVADAPNILTSSVLMAPASEDEDNVLKAFTVPKNRSLASPLQPCSSTWEPASCGKMEEQMTSSSQARKYVNAFSARTLVM

SEQ ID NO: 15 - protein, 7 aa, Artificial Sequence
  PEP
  VARIANT (4)..(4): Arg is L-Arg or D-Arg
HHHRLSH

SEQ ID NO: 16 - protein, 11 aa, Artificial Sequence
  TAT PTD
YGRKKRRQRRR

SEQ ID NO: 17 - protein, 9 aa, Artificial Sequence
  PTD
RKKRRQRRR

SEQ ID NO: 18 - protein, 7 aa, Artificial Sequence
  poly-Arg
RRRRRRR

SEQ ID NO: 19 - protein, 12 aa, Artificial Sequence
  PTD-5
RRQRRTSKLMKR

SEQ ID NO: 20 - protein, 27 aa, Artificial Sequence
  Transportan
GWTLNSAGYLLGKINLKALAALAKKIL

SEQ ID NO: 21 - protein, 29 aa, Artificial Sequence
  KALA
WEAKLAKALAKALAKHLAKALAKALKCEA

SEQ ID NO: 22 - protein, 16 aa, Artificial Sequence
  PTD
RQIKIWFQNRRMKWKK

SEQ ID NO: 23 - protein, 7 aa, Artificial Sequence
  SV 40 T antigen
PKKKRKV

SEQ ID NO: 24 - protein, 7 aa, Artificial Sequence
  NLS
GKKRSKV

SEQ ID NO: 25 - protein, 7 aa, Artificial Sequence
  NLS
KSRKRKL

SEQ ID NO: 26 - protein, 19 aa, Artificial Sequence
  NLS
KRPAATKKAGQAKKKKLDK

SEQ ID NO: 27 - protein, 20 aa, Artificial Sequence
  NLS
RKKRKTEEESPLKDKAKKSK

SEQ ID NO: 28 - protein, 20 aa, Artificial Sequence
  NLS
KDCVMNKHHRNRCQYCRLQR

SEQ ID NO: 29 - protein, 9 aa, Artificial Sequence
  NLS
PAAKRVKLD

SEQ ID NO: 30 - protein, 20 aa, Artificial Sequence
  NLS
KKYENVVIKRSPRKRGRPRK

SEQ ID NO: 31 - DNA, 21 bp, Artificial Sequence
  Pax6 Sequence 1
ccacttcaacaggactcattt

SEQ ID NO: 32 - DNA, 21 bp, Artificial Sequence
  Pax6 Sequence 2
gcaagaatacaggtatggttt

SEQ ID NO: 33 - DNA, 21 bp, Artificial Sequence
  Human Pax6 siRNA1 Forward
ggcaaucggugguaguaaatt

SEQ ID NO: 34 - DNA, 21 bp, Artificial Sequence
  Human Pax6 siRNA1 Reverse
uuuacuaccaccgauugccct

SEQ ID NO: 35 - DNA, 21 bp, Artificial Sequence
  Human Pax6 siRNA2 Forward
caagcgugucaucaauaaatt

SEQ ID NO: 36 - DNA, 21 bp, Artificial Sequence
  Human Pax6 siRNA2 Reverse
uuuauugaugacacgcuuggt